(19) United States
(12) Patent Application Publication
(10) Pub. No.: US 2011/0189162 A1
(43) Pub. Date: Aug. 4, 2011

(54) Modified Clostridial Toxins Comprising an Integrated Protease Cleavage Site-Binding Domain

(75) Inventors: Sanjiv Ghanshani, Irvine, CA (US); Linh Q. Le, Tustin, CA (US); Yi Liu, Irvine, CA (US); Lance E. Steward, Irvine, CA (US)

(73) Assignee: ALLERGAN, INC., Irvine, CA (US)

(21) Appl. No.: 12/970,239

(22) Filed: Dec. 16, 2010

Related U.S. Application Data
(60) Provisional application No. 61/286,954, filed on Dec. 16, 2009.

(51) Int. Cl. A61K 38/48; C07H 21/00; A61K 8/66; A61Q 19/08; A61P 29/00; C12N 15/63; C12N 9/52

(52) U.S. Cl. 424/94.67; 536/23.2; 435/320.1; 435/220

(57) ABSTRACT

The present specification discloses modified Clostridial toxins, compositions comprising an integrated protease cleavage site-binding domain, polynucleotide molecules encoding such modified Clostridial toxins and compositions comprising di-chain forms of such modified Clostridial toxins.

This application claims the benefit of U.S. Provisional Patent Application Ser. No. 61/286,954, filed on Dec. 16, 2009, the entire disclosure of which is incorporated herein by this specific reference.

The ability of Clostridial toxins, such as, e.g., Botulinum neurotoxins (BoNTs), BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea) BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Elan Pharmaceuticals, San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat sensory nerve-based ailments, such as, e.g., various kinds of chronic pain, neurogenic inflammation and urogentital disorders, as well as non-neuronal-based disorders, such as, e.g., pancreatitis. One approach that is currently being exploited to expand Clostridial toxin-based therapies involves modifying a Clostridial toxin so that the modified toxin has an altered cell targeting capability for a non-Clostridial toxin target cell. This re-targeted capability is achieved by replacing a naturally-occurring targeting domain of a Clostridial toxin with a targeting domain showing a selective binding activity for a non-Clostridial toxin receptor present in a non-Clostridial toxin target cell. Such modifications to a targeting domain result in a modified toxin that is able to selectively bind to a non-Clostridial toxin receptor (target receptor) present on a non-Clostridial toxin target cell (re-targeted). A re-targeted Clostridial toxin with a targeting activity for a non-Clostridial toxin target cell can bind to a receptor present on the non-Clostridial toxin target cell, translocate into the cytoplasm, and exert its proteolytic effect on the SNARE complex of the non-Clostridial toxin target cell.

Non-limiting examples of re-targeted Clostridial toxins with a targeting activity for a non-Clostridial toxin target cell are described in, e.g., Keith A. Foster et al., Clostridial Toxin Derivatives Able To Modify Peripheral Sensory Afferent Functions, U.S. Pat. No. 5,989,545 (Nov. 23, 1999); Clifford C. Shone et al., Recombinant Toxin Fragments, U.S. Pat. No. 6,461,617 (Oct. 8, 2002); Conrad P. Quinn et al., Methods and Compounds for the Treatment of Mucus Hypersecretion, U.S. Pat. No. 6,632,440 (Oct. 14, 2003); Lance E. Steward et al., Methods And Compositions For The Treatment Of Pancreatitis, U.S. Pat. No. 6,843,998 (Jan. 18, 2005); Stephan Donovan, Clostridial Toxin Derivatives and Methods For Treating Pain, U.S. Patent Publication 2002/0037833 (Mar. 28, 2002); Keith A. Foster et al., Inhibition of Secretion from Non-neural Cells, U.S. Patent Publication 2003/0180289 (Sep. 25, 2003); J. Oliver Dolly et al., Activatable Recombinant Neurotoxins, WO 2001/014570 (Mar. 1, 2001); Keith A. Foster et al., Re-targeted Toxin Conjugates, International Patent Publication WO 2005/023309 (Mar. 17, 2005); and Lance E. Steward et al., Multivalent Clostridial Toxin Derivatives and Methods of Their Use, U.S. patent application Ser. No. 11/376,696 (Mar. 15, 2006). The ability to re-target the therapeutic effects associated with Clostridial toxins has greatly extended the number of medicinal applications able to use a Clostridial toxin therapy. As a non-limiting example, modified Clostridial toxins retargeted to sensory neurons are useful in treating various kinds of chronic pain, such as, e.g., hyperalgesia and allodynia, neuropathic pain and inflammatory pain, see, e.g., Foster, supra, (1999); and Donovan, supra, (2002); and Stephan Donovan, Method For Treating Neurogenic Inflammation Pain with Botulinum Toxin and Substance P Components, U.S. Pat. No. 7,022,329 (Apr. 4, 2006). As another non-limiting example, modified Clostridial toxins retargeted to pancreatic cells are useful in treating pancreatitis, see, e.g., Steward, supra, (2005).

One surprising finding revealed during the development of re-targeted Clostridial toxins regards the placement, or presentation, of the targeting moiety. As discussed further below, naturally-occurring Clostridial toxins are organized into three major domains comprising a linear amino-to-carboxyl single polypeptide order of the enzymatic domain (amino region position), the translocation domain (middle region position) and the binding domain (carboxyl region position) (FIG. 2). This naturally-occurring order can be referred to as the carboxyl presentation of the targeting moiety because the domain necessary for binding to the cell-surface receptor is located at the carboxyl region position of the Clostridial toxin. However, it has been shown that Clostridial toxins can be modified by rearranging the linear amino-to-carboxyl single polypeptide order of the three major domains and locating a targeting moiety at the amino region position of a Clostridial toxin, referred to as amino presentation, as well as in the middle region position, referred to as central presentation (FIG. 2). While this rearrangement of the Clostridial toxin domains and location of a targeting moiety has proven successful, a problem still exists for a class of targeting moieties that require a free amino-terminus for proper receptor binding.

The problem associated with targeting moieties requiring a free amino-terminus for proper receptor binding stems from the fact that Clostridial toxins, whether naturally occurring or modified, are processed into a di-chain form in order to achieve full activity. Naturally-occurring Clostridial toxins are each translated as a single-chain polypeptide of approximately 150 kDa that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC), comprising the enzymatic domain, and an approximately 100 kDa heavy chain (HC), comprising the translocation and cell binding domains, the LC and HC being held together by the single disulfide bond and non-covalent interactions (FIG. 1). Recombinantly-produced Clostridial toxins generally substitute the naturally-occurring di-chain loop protease cleavage site with an exogenous protease cleavage site (FIG. 2). See e.g., Dolly, J. O. et al., Activatable Clostridial Toxins, U.S. Pat. No. 7,419,676 (Sep. 2, 2008), which is hereby incorporated by reference. Although re-targeted Clostridial toxins vary in their overall molecular weight because the size of the targeting moiety, the activation process and its reliance on exogenous cleavage sites is essentially the same as that for recombinantly-produced Clostridial toxins. See e.g., Steward, L. E. et al., Activatable Clostridial Toxins, U.S. patent application Ser. No. 12/192,900 (Aug. 15, 2008); Steward, L. E. et al., Modified Clostridial Toxins with Enhanced Translocation Capabilities and Altered Targeting Activity For Non-Clostridial Toxin Target Cells, U.S. patent application Ser. No. 11/776,075 (Jul. 11, 2007); Steward, L. E. et al., Modified Clostridial Toxins with Enhanced Translocation Capabilities and Altered Targeting Activity for Clostridial Toxin Target Cells, U.S. patent application Ser. No. 11/776,052 (Jul. 11, 2007), each of which is hereby incorporated by reference. In general, the activation process that converts the single-chain polypeptide into its di-chain form using exogenous proteases can be used to process re-targeted Clostridial toxins having a targeting moiety organized in an amino presentation, central presentation, or carboxyl presentation arrangement. This is because for most targeting moieties the amino-terminus of the moiety does not participate in receptor binding. As such, a wide range of protease cleavage sites can be used to produce an active di-chain form of a Clostridial toxin or re-targeted Clostridial toxin. However, targeting moieties requiring a free amino-terminus for receptor binding is an exception to this generality because, in this case, the amino-terminus of the moiety is essential for proper receptor binding. As such, a protease cleavage site whose scissile bond is not located at the carboxyl terminus of the protease cleavage site cannot be used because such sites leave a remnant of the cleavage site at the amino terminus of the targeting moiety. Thus, even though such re-targeted toxins will be processed into a di-chain form, the toxin will be inactive because of the targeting moiety's inability to bind to its cognate receptor because the cleavage site remnant masks the amino-terminal amino acid of the targeting moiety essential for receptor binding function.

For example, a retargeted Clostridial toxin comprises an amino-to-carboxyl linear order of an enzymatic domain, a human rhinovirus 3C protease cleavage site, a binding domain, and a translocation domain (a central presentation arrangement). The Human Rhinovirus 3C protease cleavage site comprises the consensus sequence P₅—P₄-L-F-Q↓-G-P—P₃′-P₄′-P₅′ (SEQ ID NO: 1), where P₅ has a preference for D or E; P₄ is G, A, V, L, I, M, S or T; and P₃′, P₄′, and P₅′ can be any amino acid. Upon cleavage of the Q-G scissile bond, the GP remnant of the cleavage site becomes the amino terminus of the targeting moiety contained within the binding domain. In general, this remnant does not interfere with binding of the targeting moiety with its cognate receptor. The one exception is a targeting moiety requiring a free amino-terminus for proper receptor binding. In this case, the GP remnant of the human rhinovirus 3C protease cleavage site masks the free amino terminus of the targeting moiety essential for proper binding, thereby inactivating the modified Clostridial toxin because of its inability to bind to its receptor and internalize into the cell.

To date, only two proteases, Factor Xa and enterokinase, have been found useful for activating re-targeted Clostridial toxins having a targeting moiety requiring a free amino-terminus for proper receptor binding. The Factor Xa cleavage site, P₅—I(E/D)GR↓-P_{1′}—P_{2′}—P_{3′}—P₄—P_{5′} (SEQ ID NO: 2), where P₅, P_{1′}, P_{2′}, P_{3′}, P_{4′}, and P_{5′} can be any amino acid, is a site-specific protease cleavage site that is cleaved at the carboxyl side of the P₁ arginine. Similarly, the enterokinase cleavage site, DDDDK↓-P_{1′}—P_{2′}—P_{3′}—P_{4′}—P_{5′}, (SEQ ID NO: 3), where P_{1′}, P_{2′}, P_{3′}, P_{4′}, and P_{5′}, can be any amino acid, is a site-specific protease cleavage site that is cleaved at the carboxyl side of the P₁ lysine. Proteolysis at either site results in a targeting moiety with its amino terminus intact because it does not leave a cleavage site remnant behind. Although other proteases may cleave at the carboxyl terminus of their cleavage site, such as, e.g., trypsin, chemotrypsin, pepsin, V8 protease, thermolysin, CNBr, Arg-C, Glu-C, Lys-C, and Tyr-C, the sites themselves are non-specific. As such, these proteases are not useful because they will cleave other regions of a retargeted toxin, thereby inactivating the toxin. However, there are several problems associated with Factor Xa and enterokinase. With regards to Factor Xa, this protease is only available as a purified product from blood-derived sources; there is currently no recombinantly-produced Factor Xa commercially available. As such, Factor Xa is unsuitable for the manufacture of a pharmaceutical drug due to health concerns over blood-derived reagents and the high cost of using such products.

Similarly, enterokinase has several disadvantages that make the manufacture of a pharmaceutical drug difficult and costly. First, enterokinase lacks current Good Manufacture Practices (cGMP) approval and seeking such approval is a time-intensive and expensive process. Second, this protease is notoriously difficult to produce recombinantly because enterokinse is a large molecule of 26.3 kDa that contains four di-sulfide bonds. As such, the use of more cost-effective bacterial-based expression systems is difficult because these systems lack the capacity to produce di-sulfide bonds. However, the use of eukaryotic-based expression systems also posses several drawbacks. One drawback is that the vast majority of recombinantly produced enterokinase is sequestered in inclusion bodies making purification of sufficient quantities of this protease difficult. Another drawback, depending on the eukaryotic cells that are used, is that additional purification steps during the manufacturing process may be required in order to meet GMP approval. Yet another drawback is that both Factor Xa and enterokinase cleave substrates at locations other than the intended target site, especially when used at higher concentrations. Thus, these problems represent a significant obstacle in the use of either Factor Xa or enterokinase for the commercial production of di-chain re-targeted Clostridial toxins comprising a targeting moiety with a free amino terminus because it is a costly, inefficient and laborious process that significantly adds to the overall cost of manufacturing such re-targeted Clostridial toxins as a biopharmaceutical drug.

The present specification discloses modified Clostridial toxin comprising a targeting moiety with a free amino terminus that do not rely on either Factor Xa or enterokinase for processing of the toxin into its di-chain form. This is accomplished by integrating a novel protease cleavage site with a targeting moiety so that after cleavage the proper amino terminus essential for receptor binding is produced.

Thus, aspects of the present invention provide a modified Clostridial toxin comprising an integrated protease cleavage site-binding domain. It is envisioned that any Clostridial toxin comprising a binding domain requiring a free amino terminus for proper receptor binding can be modified by incorporating a protease cleavage site-binding domain. Such Clostridial toxins are described in, e.g., Steward, L. E. et al., Multivalent Clostridial Toxins, U.S. patent application Ser. No. 12/210,770 (Sep. 15, 2008); Steward, L. E. et al., Activatable Clostridial Toxins, U.S. patent application Ser. No. 12/192,900 (Aug. 15, 2008); Steward, L. E. et al., Modified Clostridial Toxins with Enhanced Translocation Capabilities and Altered Targeting Activity For Non-Clostridial Toxin Target Cells, U.S. patent application Ser. No. 11/776,075 (Jul. 11, 2007); Steward, L. E. et al., Modified Clostridial Toxins with Enhanced Translocation Capabilities and Altered Targeting Activity for Clostridial Toxin Target Cells, U.S. patent application Ser. No. 11/776,052 (Jul. 11, 2007); Foster, K. A. et al., Fusion Proteins, U.S. patent application Ser. No. 11/792,210 (May 31, 2007); Foster, K. A. et al., Non-Cytotoxic Protein Conjugates, U.S. patent application Ser. No. 11/791,979 (May 31, 2007); Steward, L. E. et al., Activatable Clostridial Toxins, U.S. Patent Publication No. 2008/0032931 (Feb. 7, 2008); Foster, K. A. et al., Non-Cytotoxic Protein Conjugates, U.S. Patent Publication No. 2008/0187960 (Aug. 7, 2008); Steward, L. E. et al., Degradable Clostridial Toxins, U.S. Patent Publication No. 2008/0213830 (Sep. 4, 2008); Steward, L. E. et al., Modified Clostridial Toxins With Enhanced Translocation Capabilities and Altered Targeting Activity For Clostridial Toxin Target Cells, U.S. Patent Publication No. 2008/0241881 (Oct. 2, 2008); and Dolly, J. O. et al., Activatable Clostridial Toxins, U.S. Pat. No. 7,419,676 (Sep. 2, 2008), each of which is hereby incorporated by reference in its entirety.

Other aspects of the present invention provide polynucleotide molecules encoding a modified Clostridial toxin comprising an integrated protease cleavage site-binding domain. A polynucleotide molecule encoding a modified Clostridial toxin disclosed in the present specification can further comprise an expression vector.

Other aspects of the present invention provide a composition comprising a di-chain form of a modified Clostridial toxin disclosed in the present specification. A composition comprising a di-chain form of a modified Clostridial toxin disclosed in the present specification can be a pharmaceutical composition. Such a pharmaceutical composition can comprise, in addition to a modified Clostridial toxin disclosed in the present specification a pharmaceutical carrier, a pharmaceutical component, or both.

### BRIEF DESCRIPTION OF THE DRAWINGS

FIG. 1 shows the domain organization of naturally-occurring Clostridial toxins. The single chain form depicts the amino to carboxyl linear organization comprising an enzymatic domain, a translocation domain, and a H_{C} binding domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single chain form of the toxin into the di-chain form.

FIG. 2 shows the domain organization of Clostridial toxins arranged in the carboxyl presentation of the binding domain, the central presentation of the binding domain, and the amino presentation of the binding domain. The di-chain loop region located between the translocation and enzymatic domains is depicted by the double SS bracket. This region comprises an exogenous protease cleavage site that upon cleavage by its cognate protease converts the single-chain form of the toxin into the di-chain form.

FIG. 3 shows a schematic of the current paradigm of neurotransmitter release and Clostridial toxin intoxication in a central and peripheral neuron. FIG. 3A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 3B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the H_{N} domain of the Clostridial toxin heavy chain, separation of the Clostridial toxin light chain from the heavy chain, and release of the active light chain and 4) enzymatic target modification, where the active light chain of Clostridial toxin proteolytically cleaves its target SNARE substrate, such as, e.g., SNAP-25, VAMP or Syntaxin, thereby preventing vesicle docking and neurotransmitter release.

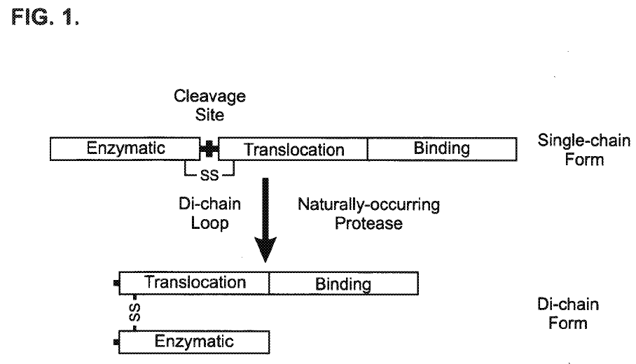

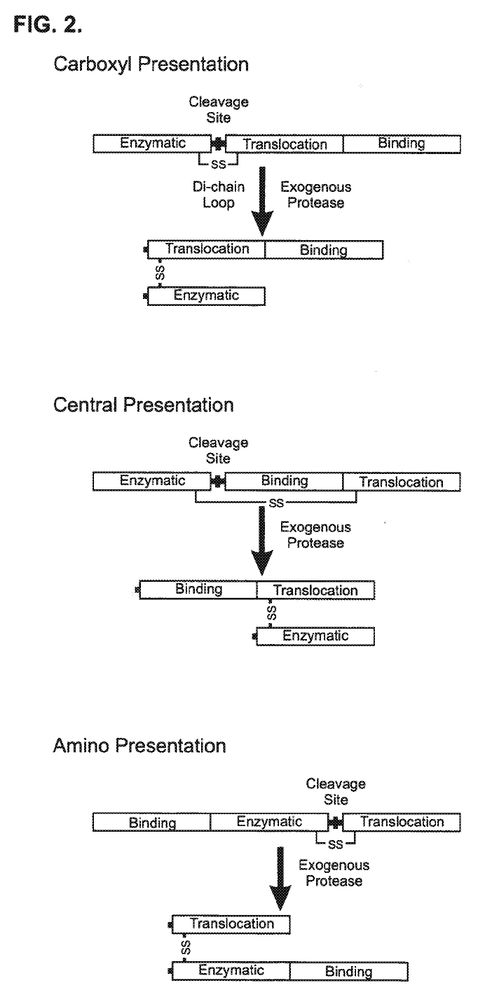

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, /B, /E and /F), animals (BoNT/C1 and /D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently four BoNT/A subtypes, BoNT/A1, BoNT/A2, BoNT/A3 and BoNT/A4, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other Clostridia species, C. baratii and C. butyricum, produce toxins, BaNT and BuNT, which are similar to BoNT/F and BoNT/E, respectively.

Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocation domain contained within the amino-terminal half of the HC (H_{N}) that facilitates release of the LC from intracellular vesicles into the cytoplasm of the target cell; and 3) a binding domain found within the carboxyl-terminal half of the HC (H_{C}) that determines the binding activity and binding specificity of the toxin to the receptor complex located at the surface of the target cell. The H_{C} domain comprises two distinct structural features of roughly equal size that indicate function and are designated the H_{CN} and H_{CC} subdomains. Table 1 gives approximate boundary regions for each domain found in exemplary Clostridial toxins.

TABLE 1
Clostridial Toxin Reference Sequences and Regions

| Toxin | SEQ ID NO: | LC | H_{N} | H_{C} |
|---|---|---|---|---|
| BoNT/A | 134 | M1-K448 | A449-K871 | N872-L1296 |
| BoNT/B | 135 | M1-K441 | A442-S858 | E859-E1291 |
| BoNT/C1 | 136 | M1-K449 | T450-N866 | N867-E1291 |
| BoNT/D | 137 | M1-R445 | D446-N862 | S863-E1276 |
| BoNT/E | 138 | M1-R422 | K423-K845 | R846-K1252 |
| BoNT/F | 139 | M1-K439 | A440-K864 | K865-E1274 |
| BoNT/G | 140 | M1-K446 | S447-S863 | N864-E1297 |
| TeNT | 141 | M1-A457 | S458-V879 | I880-D1315 |
| BaNT | 142 | M1-K431 | N432-I857 | I858-E1268 |
| BuNT | 143 | M1-R422 | K423-I847 | K848-K1251 |

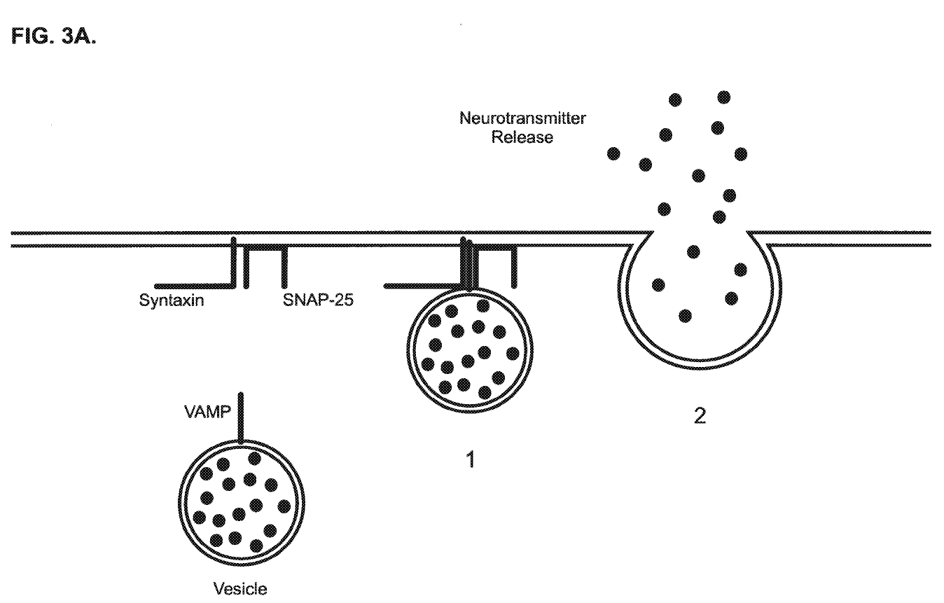

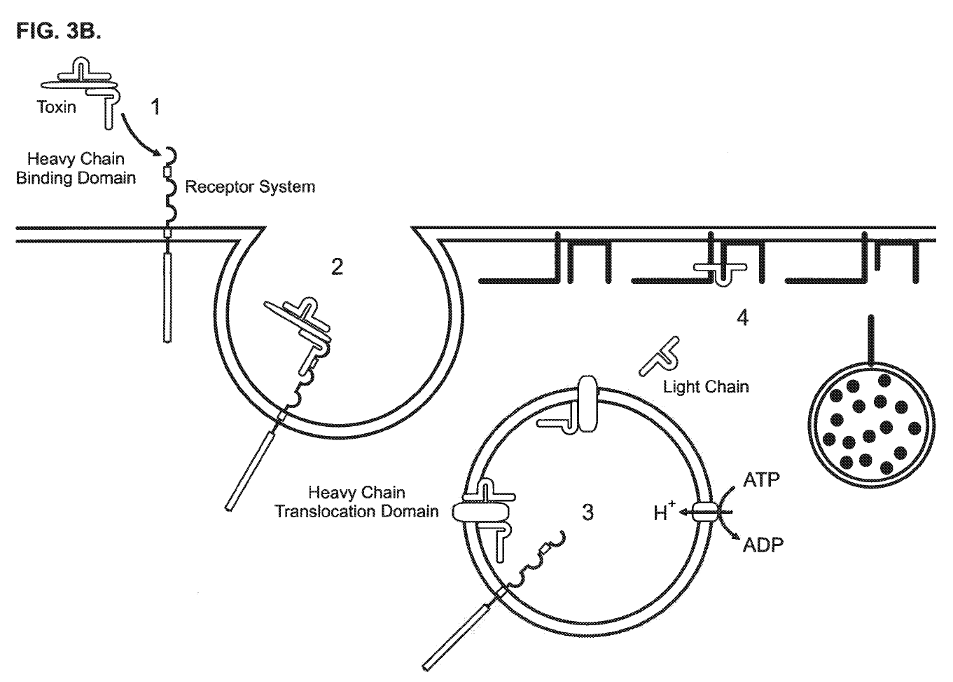

The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although the applicants have no wish to be limited by the following description, the intoxication mechanism can be described as comprising at least four steps: 1) receptor binding, 2) complex internalization, 3) light chain translocation, and 4) enzymatic target modification (FIG. 3). The process is initiated when the H_{C} domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote formation di-chain form of the toxin. Once activated, light chain endopeptidase of the toxin is released from the intracellular vesicle into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); Giovanna Lalli et al., The Journey of Tetanus and Botulinum Neurotoxins in Neurons, 11(9) Trends Microbiol. 431-437, (2003).

In an aspect of the invention, a modified Clostridial toxin comprises, in part, a single-chain modified Clostridial toxin and a di-chain modified Clostridial toxin. As discussed above, a Clostridial toxin, whether naturally-occurring or non-naturally-occurring, are initially synthesized as a single-chain polypeptide. This single-chain form is subsequently cleaved at a protease cleavage site located within a discrete di-chain loop region created between two cysteine residues that form a disulfide bridge by a protease. This posttranslational processing yields a di-chain molecule comprising a light chain (LC) and a heavy chain. As used herein, the term “di-chain loop region” refers to loop region of a naturally-occurring or non-naturally-occurring Clostridial toxin formed by a disulfide bridge located between the LC domain and the HC domain. As used herein, the term “single-chain modified Clostridial toxin” refers to any modified Clostridial toxin disclosed in the present specification that is in its single-chain form, i.e., the toxin has not been cleaved at the protease cleavage site located within the di-chain loop region by its cognate protease. As used herein, the term “di-chain modified Clostridial toxin” refers to any modified Clostridial toxin disclosed in the present specification that is in its di-chain form, i.e., the toxin has been cleaved at the protease cleavage site located within the di-chain loop region by its cognate protease.

In an aspect of the invention, a modified Clostridial toxin comprises, in part, an integrated protease cleavage site-binding domain. As used herein, the term “integrated protease cleavage site-binding domain” refers to an amino acid sequence comprising a P portion of a protease cleavage site including the P₁ site of the scissile bond and a binding domain, wherein the P₁ site of the scissile bond from the P portion of a protease cleavage site abuts the amino-end of the binding domain thereby forming an integrated protease cleavage site in which the first amino acid of the binding domain serves as the P₁′ site of the scissile bond. As described in greater detail below, the P portion of a protease cleavage site refers to an amino acid sequence taken from the P portion (≧P₆—P₅—P₄—P₃—P₂—P₁) of the canonical consensus sequence of a protease cleavage site (≧P₆—P₅—P₄—P₃—P₂—P₁—P₁′-P₂′-P₃′-P₄′-P₅′-≧P₆′, where P₁—P₁′ is the scissile bond). As such, the amino-terminal amino acid of the binding domain serves both in the formation of a scissile bond and as the first residue of the binding domain that is essential for proper binding of the binding domain to its cognate receptor. Non-limiting examples of integrated protease cleavage site-binding domains are listed in Table 2. It is known in the art that when locating an integrated protease cleavage site-binding domain at the amino terminus of the modified Clostridial toxin (amino presentation), a start methionine should be added to maximize expression of the modified Clostridial toxin. In addition, the P portion of a protease cleavage site including the P₁ site of the scissile bond of SEQ ID NO: 127, or the P portion of a protease cleavage site including the P₁ site of the scissile bond of SEQ ID NO: 130, can replace the P portion of a protease cleavage site including the P₁ site of the scissile bond of SEQ ID NO: 121 present in the protease integrated protease cleavage site-binding domains listed in Table 2.

TABLE 2
Integrated Protease Cleavage Site-Binding Domaine

| | | SEQ |
|---|---|---|
| | Integrated Protease Cleavage | ID |
| Targeting Moiety | Site-Targetiog Moiety | NO: |
| Leu-enkephalin | EXXYXQYGGFL | 4 |
| Met-enkephalin | EXXYXQYGGFM | 5 |
| Met-enkephalin MRGL | EXXYXQYGGFMRGL | 6 |
| Met-enkephalin MRF | EXXYXQYGGFMRF | 7 |
| BAM-22 (1-12) | EXXYXQYGGFMRRVGRPE | 8 |
| BAM-22 (1-12) | EXXYXQYGGFMRRVGRPD | 9 |
| BAM-22 (6-22) | EXXYXQRVGRPEWWMDYQKRYG | 10 |
| BAM-22 (6-22) | EXXYXQRVGRPEWWLDYQKRTG | 11 |
| BAM-22 (6-22) | EXXYXQRVGRPEWWQDYQKRYG | 12 |
| BAM-22 (6-22) | EXXYXQRVGRPEWWEDYQKRYG | 13 |
| BAM-22 (6-22) | EXXYXQRVGRPEWKLDNQKRYG | 14 |
| BAM-22 (6-22) | EXXYXQRVGRPDWWQESKRYG | 15 |
| BAM-22 (8-22) | EXXYXQGRPEWWMDYQKRYG | 16 |
| BAM-22 (8-22) | EXXYXQGRPEWWLDYQKRTG | 17 |
| BAM-22 (8-22) | EXXYXQGRPEWWQDYQKRYG | 18 |
| BAM-22 (8-22) | EXXYXQGRPEWWEDYQKRYG | 19 |
| BAM-22 (8-22) | EXXYXQGRPEWKLDNQKRYG | 20 |
| BAM-22 (8-22) | EXXYXQGRPDWWQESKRYG | 21 |
| BAM-22 (1-22) | EXXYXQYGGFMRRVGRPEWWMDYQKRYG | 22 |
| BAM-22 (1-22) | EXXYXQYGGFMRRVGRPEWWLDYQKRTG | 23 |
| BAM-22 (1-22) | EXXYXQYGGFMRRVGRPEWWQDYQKRYG | 24 |
| BAM-22 (1-22) | EXXYXQYGGFMRRVGRPEWWEDYQKRYG | 25 |
| BAM-22 (1-22) | EXXYXQYGGFMRRVGRPEWKLDNQKRYG | 26 |
| BAM-22 (1-22) | EXXYXQYGGFMRRVGRPDWWQESKRYG | 27 |
| Endomorphin-1 | EXXYXQYPYF | 28 |
| Endomorphin-2 | EXXYXQYPFF | 29 |
| Endorphin-α | EXXYXQYGGFMTSEKSQTPLVT | 30 |
| Neoendorphin-α | EXXYXQYGGFLRKYPK | 31 |
| Endorphin-β | EXXYXQYGGFMTSEKSQTPLVTLFKNAIIKNAYKKGE | 32 |
| Endorphin-β | EXXYXQYGGFMSSEKSQTPLVTLFKNAIIKNAHKKGQ | 33 |
| Neoendorphin-β | EXXYXQYGGFLRKYP | 34 |
| Endorphin-γ | EXXYXQYGGFMTSEKSQTPLVTL | 35 |
| Dynorphin A (1-17) | EXXYXQYGGFLRRIRPKLKWDNQ | 36 |
| Dynorphin A (1-13) | EXXYXQYGGFLRRIRPKLK | 37 |
| Dynorphin A (2-17) | EXXYXQGGFLRRIRPKLKWDNQ | 38 |
| Dynorphin A (2-13) | EXXYXQGGFLRRIRPKLK | 39 |
| Dynorphin A (1-17) | EXXYXQYGGFLRRIRPKLRWDNQ | 40 |
| Dynorphin A (1-13) | EXXYXQYGGFLRRIRPKLR | 41 |
| Dynorphin A (1-17) | EXXYXQYGGFLRRIRPRLRWDNQ | 42 |
| Dynorphin A (1-13) | EXXYXQYGGFLRRIRPRLR | 43 |
| Dynorphin A (1-17) | EXXYXQYGGFMRRIRPKLRWDNQ | 44 |
| Dynorphin A (1-13) | EXXYXQYGGFMRRIRPKLR | 45 |
| Dynorphin A (1-17) | EXXYXQYGGFMRRIRPKIRWDNQ | 46 |
| Dynorphin A (1-13) | EXXYXQYGGFMRRIRPKIR | 47 |
| Dynorphin A (1-17) | EXXYXQYGGFMRRIRPKLKWDSQ | 48 |
| Dynorphin A (1-13) | EXXYXQYGGFMRRIRPKLK | 49 |
| Dynorphin A (1-9) | EXXYXQYGGFLRRIR | 50 |
| Dynorphin A (1-9) | EXXYXQYGGFMRRIR | 51 |
| Dynorphin B | EXXYXQYGGFLRRQFKVVTRSQEDPNAYSGELFDA | 52 |
| Dynorphin B | EXXYXQYGGFLRRQFKVVTRSQENPNTYSEDLDV | 53 |
| Dynorphin B | EXXYXQYGGFLRRQFKVVTRSQESPNTYSEDLDV | 54 |
| Dynorphin B | EXXYXQYGGFLRRQFKVVTRSQEDPNAYSEEFFDV | 55 |
| Dynorphin B | EXXYXQYGGFLRRQFKVVTRSQEDPNAYYEELFDV | 56 |
| Dynorphin B | EXXYXQYGGFLRRQFKVVTRSQEDPNAYSGELLDG | 57 |
| Dynorphin B | EXXYXQYGGFLRRQFKVVTRSQEDPSAYYEELFDV | 58 |
| Dynorphin B | EXXYXQYGGFLRRQFKVTTRSEEDPSTFSGELSNL | 59 |
| Dynorphin B | EXXYXQYGGFLRRQFKVTTRSEEEPGSFSGEISNL | 60 |
| Dynorphin B | EXXYXQYGGFLRRQFKVNARSEEDPTMFSDELSYL | 61 |
| Dynorphin B | EXXYXQYGGFLRRQFKVNARSEEDPTMFSGELSYL | 62 |
| Dynorphin B | EXXYXQYGGFLRRHFKISVRSDEEPSSYSDEVLEL | 63 |
| Dynorphin B | EXXYXQYGGFLRRHFKITVRSDEDPSPYLDEFSDL | 64 |
| Dynorphin B | EXXYXQYGGFLRRHFKISVRSDEEPSSYEDYAL | 65 |
| Dynorphin B | EXXYXQYGGFLRRHFKISVRSDEEPGSYDVIGL | 66 |
| Dynorphin B | EXXYXQYGGFLRRHYKLSVRSDEEPSSYDDFGL | 67 |
| Dynorphin B (1-7) | EXXYXQYGGFLRR | 68 |
| Rimorphin | EXXYXQYGGFLRRQFKVVT | 69 |
| Rimorphin | EXXYXQYGGFLRRQFKVTT | 70 |
| Rimorphin | EXXYXQYGGFLRRQFKVNA | 71 |
| Rimorphin | EXXYXQYGGFLRRHFKISV | 72 |
| Rimorphin | EXXYXQYGGFLRRHFKITV | 73 |
| Rimorphin | EXXYXQYGGFLRRHYKLSV | 74 |
| Nociceptin (1-17) | EXXYXQFGGFTGARKSARKRKNQ | 75 |
| Nociceptin (1-17) | EXXYXQFGGFYGARKSARKLANQ | 76 |
| Nociceptin (1-17) | EXXYXQFGGFTGARKSARKYANQ | 77 |
| Nociceptin (1-13) | EXXYXQFGGFTGARKSARK | 78 |
| Nociceptin (1-11) | EXXYXQFGGFTGARKYARK | 79 |
| Nociceptin (1-11) | EXXYXQFGGFTGARKSYRK | 80 |
| Nociceptin (1-11) | EXXYXQFGGFTGARKSA | 81 |
| Nociceptin (1-11) | EXXYXQFGGFTGARKYA | 82 |
| Nociceptin (1-11) | EXXYXQFGGFTGARKSY | 83 |
| Nociceptin (1-9) | EXXYXQFGGFTGARK | 84 |
| Neuropeptide 1 | EXXYXQMPRVRSLFQEQEEPEPGMEEAGEMEQKQLQ | 85 |
| Neuropeptide 2 | EXXYXQFSEFMRQYLVLSMQSSQ | 86 |
| Neuropeptide 3 | EXXYXQTLHQNGNV | 87 |
| PAR 1 | EXXYXQSFLLRN | 88 |
| PAR 1 | EXXYXQSFFLRN | 89 |
| PAR 1 | EXXYXQSFFLKN | 90 |
| PAR 1 | EXXYXQTFLLRN | 91 |
| PAR 1 | EXXYXQGFPGKF | 92 |
| PAR 1 | EXXYXQGYPAKF | 93 |
| PAR 1 | EXXYXQGYPLKF | 94 |
| PAR 1 | EXXYXQGYPIKF | 95 |
| PAR 2 | EXXYXQSLIGKV | 96 |
| PAR 2 | EXXYXQSLIGRL | 97 |
| PAR 3 | EXXYXQTFRGAP | 98 |
| PAR 3 | EXXYXQSFNGGP | 99 |
| PAR 3 | EXXYXQSFNGNE | 100 |
| PAR 4 | EXXYXQGYPGQV | 101 |
| PAR 4 | EXXYXQAYPGKF | 102 |
| PAR 4 | EXXYXQTYPGKF | 103 |
| PAR 4 | EXXYXQGYPGKY | 104 |
| PAR 4 | EXXYXQGYPGKW | 105 |
| PAR 4 | EXXYXQGYPGKK | 106 |
| PAR 4 | EXXYXQGYPGKF | 107 |
| PAR 4 | EXXYXQGYPGRF | 108 |
| PAR 4 | EXXYXQGYPGFK | 109 |
| PAR 4 | EXXYXQGYPAKF | 110 |
| PAR 4 | EXXYXQGFPGKF | 111 |
| PAR 4 | EXXYXQGFPGKP | 112 |
| PAR 4 | EXXYXQSYPGKF | 113 |
| PAR 4 | EXXYXQSYPAKF | 114 |
| PAR 4 | EXXYXQSYPGRF | 115 |
| PAR 4 | EXXYXQSYAGKF | 116 |
| PAR 4 | EXXYXQSFPGQP | 117 |
| PAR 4 | EXXYXQSFPGQA | 118 |
| Galanin (1-30) | EXXYXQGWTLNSAGYLLGPHAVGNHRSFSDKNGLTS | 191 |
| Galanin (1-20) | EXXYXQGWTLNSAGYLLGPHAVGNHR | 192 |
| Galanin (1-16) | EXXYXQGWTLNSAGYLLGPHAV | 193 |
| Galanin (1-15) | EXXYXQGWTLNSAGYLLGPHA | 194 |
| Galanin (1-14) | EXXYXQGWTLNSAGYLLGPH | 195 |
| Galanin (1-12) | EXXYXQGWTLNSAGYLLG | 196 |
| Galanin (2-30) | EXXYXQWTLNSAGYLLGPHAVGNHRSFSDKNGLTS | 197 |
| Galanin (3-30) | EXXYXQLNSAGYLLGPHAVGNHRSFSDKNGLTS | 198 |

It is envisioned that any P portion of a protease cleavage site including the P₁ site of the scissile bond can be used, in conjunction with a binding domain, to form an integrated protease cleavage site as part of an integrated protease cleavage site-binding domain disclosed in the present invention, with the proviso that the resulting integrated protease cleavage site is selectively recognized by a protease, and, upon proteolytic cleavage, the resulting amino terminus of the binding domain is capable of selectively binding to its cognate receptor. As used herein, the term “selectively recognized by a protease” refers to the ability of a protease to recognize an integrated protease cleavage site with the same or substantially the same level of recognition as the intact protease cleavage site, i.e., the canonical consensus sequence or a protease cleavage site that does not have removed the P′ portion of the protease cleavage site including the P₁′ portion. In an aspect of this embodiment, a protease selectively recognizes an integrated protease cleavage site when protease recognition of the integrated protease cleavage site is, e.g., at least 10% the recognition level of the intact protease cleavage site, at least 20% the recognition level of the intact protease cleavage site, at least 30% the recognition level of the intact protease cleavage site, at least 40% the recognition level of the intact protease cleavage site, at least 50% the recognition level of the intact protease cleavage site, at least 60% the recognition level of the intact protease cleavage site, at least 70% the recognition level of the intact protease cleavage site, at least 80% the recognition level of the intact protease cleavage site, at least 90% the recognition level of the intact protease cleavage site, at least 95% the recognition level of the intact protease cleavage site, or 100% the recognition level of the intact protease cleavage site.

In another aspect of this embodiment, a protease selectively recognizes an integrated protease cleavage site when protease recognition of the integrated protease cleavage site is from, e.g., 10% to 100% the recognition level of the intact protease cleavage site, 10% to 90% the recognition level of the intact protease cleavage site, 10% to 80% the recognition level of the intact protease cleavage site, 10% to 70% the recognition level of the intact protease cleavage site, 20% to 100% the recognition level of the intact protease cleavage site, 20% to 90% the recognition level of the intact protease cleavage site, 20% to 80% the recognition level of the intact protease cleavage site, 20% to 70% the recognition level of the intact protease cleavage site, 30% to 100% the recognition level of the intact protease cleavage site, 30% to 90% the recognition level of the intact protease cleavage site, 30% to 80% the recognition level of the intact protease cleavage site, 30% to 70% the recognition level of the intact protease cleavage site, 40% to 100% the recognition level of the intact protease cleavage site, 40% to 90% the recognition level of the intact protease cleavage site, 40% to 80% the recognition level of the intact protease cleavage site, 40% to 70% the recognition level of the intact protease cleavage site, 50% to 100% the recognition level of the intact protease cleavage site, 50% to 90% the recognition level of the intact protease cleavage site, 50% to 80% the recognition level of the intact protease cleavage site, or 50% to 70% the recognition level of the intact protease cleavage site.

In another aspect, the protease can recognize an integrated protease cleavage site with the same or substantially the same level of binding affinity as the intact protease cleavage site, i.e., the canonical consensus sequence or a protease cleavage site that does not have removed the P′ portion of the protease cleavage site including the P₁′ portion. In an aspect of this embodiment, a protease selectively recognizes an integrated protease cleavage site when the binding affinity of the protease for the integrated protease cleavage site-binding domain is, e.g., at least 10% the binding affinity for the intact protease cleavage site, at least 20% the binding affinity for the intact protease cleavage site, at least 30% the binding affinity for the intact protease cleavage site, at least 40% the binding affinity for the intact protease cleavage site, at least 50% the binding affinity for the intact protease cleavage site, at least 60% the binding affinity for the intact protease cleavage site, at least 70% the binding affinity for the intact protease cleavage site, at least 80% the binding affinity for the intact protease cleavage site, at least 90% the binding affinity for the intact protease cleavage site, at least 95% the binding affinity for the intact protease cleavage site, or 100% the binding affinity for the intact protease cleavage site.

In another aspect of this embodiment, a protease selectively recognizes an integrated protease cleavage site when the binding affinity of the protease for the integrated protease cleavage site-binding domain is from, e.g., 10% to 100% the binding affinity for the intact protease cleavage site, 10% to 90% the binding affinity for the intact protease cleavage site, 10% to 80% the binding affinity for the intact protease cleavage site, 10% to 70% the binding affinity for the intact protease cleavage site, 20% to 100% the binding affinity for the intact protease cleavage site, 20% to 90% the binding affinity for the intact protease cleavage site, 20% to 80% the binding affinity for the intact protease cleavage site, 20% to 70% the binding affinity for the intact protease cleavage site, 30% to 100% the binding affinity for the intact protease cleavage site, 30% to 90% the binding affinity for the intact protease cleavage site, 30% to 80% the binding affinity for the intact protease cleavage site, 30% to 70% the binding affinity for the intact protease cleavage site, 40% to 100% the binding affinity for the intact protease cleavage site, 40% to 90% the binding affinity for the intact protease cleavage site, 40% to 80% the binding affinity for the intact protease cleavage site, 40% to 70% the binding affinity for the intact protease cleavage site, 50% to 100% the binding affinity for the intact protease cleavage site, 50% to 90% the binding affinity for the intact protease cleavage site, 50% to 80% the binding affinity for the intact protease cleavage site, or 50% to 70% the binding affinity for the intact protease cleavage site.

In another aspect, the protease can recognize an integrated protease cleavage site with the same or substantially the same level of cleavage efficiency as the intact protease cleavage site, i.e., the canonical consensus sequence or a protease cleavage site that does not have removed the P′ portion of the protease cleavage site including the P₁′ portion. In an aspect of this embodiment, a protease selectively recognizes an integrated protease cleavage site when the protease's cleavage efficiency for the integrated protease cleavage site-binding domain is, e.g., at least 10% the cleavage efficiency for the intact protease cleavage site, at least 20% the cleavage efficiency for the intact protease cleavage site, at least 30% the cleavage efficiency for the intact protease cleavage site, at least 40% the cleavage efficiency for the intact protease cleavage site, at least 50% the cleavage efficiency for the intact protease cleavage site, at least 60% the cleavage efficiency for the intact protease cleavage site, at least 70% the cleavage efficiency for the intact protease cleavage site, at least 80% the cleavage efficiency for the intact protease cleavage site, at least 90% the cleavage efficiency for the intact protease cleavage site, at least 95% the cleavage efficiency for the intact protease cleavage site, or 100% the cleavage efficiency for the intact protease cleavage site.

In another aspect of this embodiment, a protease selectively recognizes an integrated protease cleavage site when the protease's cleavage efficiency for the integrated protease cleavage site-binding domain is from, e.g., 10% to 100% the cleavage efficiency for the intact protease cleavage site, 10% to 90% the cleavage efficiency for the intact protease cleavage site, 10% to 80% the cleavage efficiency for the intact protease cleavage site, 10% to 70% the cleavage efficiency for the intact protease cleavage site, 20% to 100% the cleavage efficiency for the intact protease cleavage site, 20% to 90% the cleavage efficiency for the intact protease cleavage site, 20% to 80% the cleavage efficiency for the intact protease cleavage site, 20% to 70% the cleavage efficiency for the intact protease cleavage site, 30% to 100% the cleavage efficiency for the intact protease cleavage site, 30% to 90% the cleavage efficiency for the intact protease cleavage site, 30% to 80% the cleavage efficiency for the intact protease cleavage site, 30% to 70% the cleavage efficiency for the intact protease cleavage site, 40% to 100% the cleavage efficiency for the intact protease cleavage site, 40% to 90% the cleavage efficiency for the intact protease cleavage site, 40% to 80% the cleavage efficiency for the intact protease cleavage site, 40% to 70% the cleavage efficiency for the intact protease cleavage site, 50% to 100% the cleavage efficiency for the intact protease cleavage site, 50% to 90% the cleavage efficiency for the intact protease cleavage site, 50% to 80% the cleavage efficiency for the intact protease cleavage site, or 50% to 70% the cleavage efficiency for the intact protease cleavage site.

In an aspect of the invention, a modified Clostridial toxin comprises, in part, a P portion of a protease cleavage site including the P₁ site of the scissile bond. The canonical consensus sequence of a protease cleavage site can be denoted as ≧P₆—P₅—P₄—P₃—P₂—P₁—P₁′-P₂′-P₃′-P₄′-P₅′-≧P₆′, where P₁—P₁′ is the scissile bond. As used herein, the term “P portion of a protease cleavage site including the P₁ site of the scissile bond” refers to an amino acid sequence taken from the P portion (≧P₆—P₅—P₄—P₃—P₂—P₁) of the canonical consensus sequence that comprises the P₁ site of the scissile bond, such as, e.g., the amino acid sequences P₁, P₂—P₁, P₃—P₂—P₁, P₄—P₃—P₂—P₁, or P₅—P₄—P₃—P₂—P₁. As used herein, the term “P′ portion of a protease cleavage site including the P₁′ site of the scissile bond” refers to an amino acid sequence taken from the P′ portion (P₁′-P₂′-P₃′-P₄′-P₅-≧P₆′) of the canonical consensus sequence that comprises the P₁′ site of the scissile bond, such as, e.g., the amino acid sequences P₁′, P₁′-P₂′, P₁′-P₂′-P₃′, P₁′-P₂′—P₃′-P₄′, or P₁′-P₂′-P₃′-P₄′—P₅′.

For site-specific proteases the majority of the amino acids present in this P₅—P₄—P₃—P₂—P₁—P₁′-P₂′-P₃′-P₄′-P₅′ cleavage site sequence are highly conserved. Thus, for example, Human Rhinovirus 3C has a consensus sequence of P₅—P₄-L-F-Q-G-P—P₃′-P₄′-P₅′, (SEQ ID NO: 1) with a preference for D or E at the P₅ position; G, A, V, L, I, M, S or T at the P₄ position; L at the P₃ position; F at the P₂ position; Q at the P₁ position; G at the P_{1′}, position; and P at the P₂′ position. Because this high sequence conservation is required for cleavage specificity or selectivity, alteration of the consensus sequence usually results in a site that cannot be cleaved by its cognate protease. For example, removal of the five residues on the carboxyl-terminal side of the scissile bond from Human Rhinovirus 3C protease (cleavage site (G-P—P₃′-P₄′-P₅′, SEQ ID NO: 119) creates a cleavage site comprising only P₅—P₄-L-F-Q (SEQ ID NO: 120) which cannot be cleaved by this protease. One important aspect of the present invention is the finding that certain protease cleavage sites can be altered by removing the P′ portion of a protease cleavage site including the P₁′ site of the scissile bond, and yet still be specifically or selectively recognized by its cognate protease.

Thus, in one embodiment, the P portion of a protease cleavage site is the P₁ site of the scissile bond. In aspects of this embodiment, the P portion of a protease cleavage site including the P₁ site of the scissile bond is, e.g., a P₂—P₁ sequence, a P₃—P₂—P₁ sequence, a P₄—P₃—P₂—P₁ sequence, a P₅—P₄—P₃—P₂—P₁ sequence, or an amino acid fragment including a P₅—P₄—P₃—P₂—P₁ sequence and extending beyond this sequence in an amino direction, i.e., ≧P₆. In another embodiment, the P′ portion of the protease cleavage site including the P₁′ site of the scissile bond removed is a P₁′ site. In aspects of this embodiment, the P′ portion of the protease cleavage site including the P₁′ site of the scissile bond removed is, e.g., a P₁′-P₂′ sequence, a P₁′-P₂′-P₃′ sequence, a P₁′-P₂′-P₃′-P₄′ sequence, a P₁′-P₂′-P₃′-P₄′-P₅′ sequence, or an amino acid fragment including a P₁′-P₂′-P₃′-P₄′-P₅′ sequence and extending beyond this sequence in an carboxyl direction, i.e., ≧P₆′.

In an aspect of this embodiment, a P portion of a protease cleavage site including the P₁ site of the scissile bond comprises the consensus sequence E-P₅—P₄—Y—P₂-Q* (SEQ ID NO: 121), where P₂, P₄ and P₅ can be any amino acid. In other aspects of the embodiment, an integrated protease cleavage site is SEQ ID NO: 122, SEQ ID NO: 123, SEQ ID NO: 124, SEQ ID NO: 125, or SEQ ID NO: 126 (Table 3). In another aspect of this embodiment, a P portion of a protease cleavage site including the P₁ site of the scissile bond comprises the consensus sequence P₅—V—R—F-Q* (SEQ ID NO: 127), where P₅ can be any amino acid. In other aspects of the embodiment, an integrated protease cleavage site is SEQ ID NO: 128, or SEQ ID NO: 129 (Table 3). In another aspect of this embodiment, a P portion of a protease cleavage site including the P₁ site of the scissile bond comprises the consensus sequence P₅-D-P₃—P₂-D* (SEQ ID NO: 130), where P₅ can be any amino acid; P₃ can be any amino acid, with E preferred; and P₂ can be any amino acid. In other aspects of the embodiment, an integrated protease cleavage site is SEQ ID NO: 131, SEQ ID NO: 132 or SEQ ID NO: 133 (Table 3).

TABLE 3
Examples of a P portion of a protease cleavage
site including the P₁ site of the scissile bond

| | Non-limiting | SEQ ID |
|---|---|---|
| Protease Cleavage Site Consensus Sequence | Examples | NO: |
| E P₅ P₄YP₂Q* (SEQ ID NO: 121), where P₂, | ENLYFQ* | 122 |
| P₄ and P₅ can be any amino acid | ENIYTQ* | 123 |
| | ENIYLQ* | 124 |
| | ENVYFQ* | 125 |
| | ENVYSQ* | 126 |
| P₅-V-R-F-Q* (SEQ ID NO: 127), where P₅ | TVRFQ* | 128 |
| can be any amino acid | NVRFQ* | 129 |
| P₅-D-P₃-P²-D* (SEQ ID NO: 130), where P₅ | LDEVD* | 131 |
| can be any amino acid, P₃ can be any amino | VDEPD* | 132 |
| acid,with E preferred, and P₂ can be any | | |
| amino acid | VDELD* | 133 |

An asterisks (*) indicates the peptide bond that is cleaved by the indicated protease.

In an aspect of the invention, a modified Clostridial toxin comprises, in part, a binding domain. As used herein, the term “binding domain” is synonymous with “targeting moiety,” and refers to an amino acid sequence region that preferentially binds to a cell surface marker characteristic of the target cell under physiological conditions. The cell surface marker may comprise a polypeptide, a polysaccharide, a lipid, a glycoprotein, a lipoprotein, or may have structural characteristics of more than one of these. As used herein, the term “preferentially binds” refers to the ability of a binding domain to bind to its cell surface marker with at least one order of magnitude difference form that of the binding domain for any other cell surface marker. In aspects of this embodiment, a binding domain preferential binds to a cell surface marker when the disassociation constant (K_{d}) is e.g., at least 1 order of magnitude less than that of the binding domain for any other cell surface marker, at least 2 orders of magnitude less than that of the binding domain for any other cell surface marker, at least 3 orders of magnitude less than that of the binding domain for any other cell surface marker, at least 4 orders of magnitude less than that of the binding domain for any other cell surface marker, or at least 5 orders of magnitude less than that of the binding domain for any other cell surface marker. In other aspects of this embodiment, a binding domain preferential binds to a cell surface marker when the disassociation constant (K_{d}) is e.g., at most 1×10⁻⁵ M⁻¹, at most 1×10⁻⁶ M⁻¹, at most 1×10⁻⁷ M⁻¹, at most 1×10⁻⁸ M⁻¹, at most 1×10⁻⁹ M⁻¹, at most 1×10⁻¹⁰ M⁻¹, at most 1×10⁻¹¹ M⁻¹, or at most 1×10⁻¹⁰ M⁻¹²

In yet other aspects of this embodiment, a binding domain preferential binds to a cell surface marker when the association constant (Kₐ) is e.g., at least 1 order of magnitude more than that of the binding domain for any other cell surface marker, at least 2 orders of magnitude more than that of the binding domain for any other cell surface marker, at least 3 orders of magnitude more than that of the binding domain for any other cell surface marker, at least 4 orders of magnitude more than that of the binding domain for any other cell surface marker, or at least 5 orders of magnitude more than that of the binding domain for any other cell surface marker. In further aspects of this embodiment, a binding domain preferentially binds to a cell surface marker when the association constant (Kₐ) is e.g., at least 1×10⁻⁵ M⁻¹, at least 1×10⁻⁶ M⁻¹, at least 1×10⁻⁷ M⁻¹, at least 1×10⁻⁸ M⁻¹, at least 1×10⁻⁹ M⁻¹, or at least 1×10⁻¹⁰ M⁻¹.

It is envisioned that any binding domain can be used as part of an integrated protease cleavage site-binding domain disclosed in the present invention. Examples of binding domains requiring a free amino terminus for receptor binding that can be used as part of an integrated protease cleavage site-binding domain disclosed in the present invention are described in, e.g., Steward, U.S. patent application Ser. No. 12/210,770, supra, (2008); Steward, U.S. patent application Ser. No. 12/192,900, supra, (2008); Steward, U.S. patent application Ser. No. 11/776,075, supra, (2007); Steward, U.S. patent application Ser. No. 11/776,052, supra, (2007); Foster, U.S. patent application Ser. No. 11/792,210, supra, (2007); Foster, U.S. patent application Ser. No. 11/791,979, supra, (2007); Steward, U.S. Patent Publication No. 2008/0032931, supra, (2008); Foster, U.S. Patent Publication No. 2008/0187960, supra, (2008); Steward, U.S. Patent Publication No. 2008/0213830, supra, (2008); Steward, U.S. Patent Publication No. 2008/0241881, supra, (2008); and Dolly, U.S. Pat. No. 7,419,676, supra, (2008), each of which is hereby incorporated by reference in its entirety. Non-limiting examples of such binding domains, include opioids, such as, e.g., an enkephalin, an endomorphin, an endorphin, a dynorphin, a nociceptin, a rimorphin, or a functional derivatives of such opioids, and protease activated receptor (PAR) ligands.

In aspects of this embodiment, an enkephalin useful as a binding domain is a Leu-enkephalin, a Met-enkephalin, a Met-enkephalin MRGL, a Met-enkephalin MRF, or a functional derivative of such enkephalins. In other aspects of this embodiment, a BAM22 useful as a binding domain is a BAM22 peptide (1-12), a BAM22 peptide (6-22), a BAM22 peptide (8-22), a BAM22 peptide (1-22), or a functional derivative of such BAM22s. In aspects of this embodiment, an endomorphin useful as a binding domain is an endomorphin-1, an endomorphin-2, or a functional derivative of such endomorphins. In yet other aspects of this embodiment, an endorphin useful as a binding domain is an endorphin-α, a neoendorphin-α, an endorphin-β, a neoendorphin-β, an endorphin-γ, or a functional derivative of such endorphins. In still other aspects of this embodiment, a dynorphin useful as a binding domain is a dynorphin A, a dynorphin B (leumorphin), a rimorphin, or a functional derivative of such dynorphins. In further aspects of this embodiment, a nociceptin useful as a binding domain is a nociceptin RK, a nociceptin, a neuropeptide 1, a neuropeptide 2, a neuropeptide 3, or a functional derivative of such nociceptins. In yet further aspects of this embodiment, a PAR ligand useful as a binding domain is a PAR1, a PAR2, a PAR3, a PAR4, or a functional derivative of such PAR ligands.

In other aspects of this embodiment, a binding domain is any one of SEQ ID NO: 154 through SEQ ID NO: 186. In other aspects of this embodiment, a binding domain has, e.g., at least 70% amino acid identity with any one of SEQ ID NO: 154 through SEQ ID NO: 186, at least 75% amino acid identity with any one of SEQ ID NO: 154 through SEQ ID NO: 186, at least 80% amino acid identity with any one of SEQ ID NO: 154 through SEQ ID NO: 186, at least 85% amino acid identity with any one of SEQ ID NO: 154 through SEQ ID NO: 186, at least 90% amino acid identity with any one of SEQ ID NO: 154 through SEQ ID NO: 186 or at least 95% amino acid identity with any one of SEQ ID NO: 154 through SEQ ID NO: 186. In yet other aspects of this embodiment, a binding domain has, e.g., at most 70% amino acid identity with any one of SEQ ID NO: 154 through SEQ ID NO: 186, at most 75% amino acid identity with any one of SEQ ID NO: 154 through SEQ ID NO: 186, at most 80% amino acid identity with any one of SEQ ID NO: 154 through SEQ ID NO: 186, at most 85% amino acid identity with any one of SEQ ID NO: 154 through SEQ ID NO: 186, at most 90% amino acid identity with any one of SEQ ID NO: 154 through SEQ ID NO: 186 or at most 95% amino acid identity with any one of SEQ ID NO: 154 through SEQ ID NO: 186.

In other aspects of this embodiment, a binding domain has, e.g., at least one, two or three non-contiguous amino acid substitutions relative to any one of SEQ ID NO: 154 through SEQ ID NO: 186. In other aspects of this embodiment, a binding domain has, e.g., at most one, two or three non-contiguous amino acid substitutions relative to any one of SEQ ID NO: 154 through SEQ ID NO: 186. In yet other aspects of this embodiment, a binding domain has, e.g., at least one, two or three non-contiguous amino acid deletions relative to any one of SEQ ID NO: 154 through SEQ ID NO: 186. In yet other aspects of this embodiment, a binding domain has, e.g., at most one, two or three non-contiguous amino acid deletions relative to any one of SEQ ID NO: 154 through SEQ ID NO: 186. In still other aspects of this embodiment, a binding domain has, e.g., at least one, two or three non-contiguous amino acid additions relative to any one of SEQ ID NO: 154 through SEQ ID NO: 186. In yet other aspects of this embodiment, a binding domain has, e.g., at most one, two or three non-contiguous amino acid additions relative to any one of SEQ ID NO: 154 through SEQ ID NO: 186.

In other aspects of this embodiment, a binding domain has, e.g., at least one, two or three contiguous amino acid substitutions relative to any one of SEQ ID NO: 154 through SEQ ID NO: 186. In other aspects of this embodiment, a binding domain has, e.g., at most one, two or three contiguous amino acid substitutions relative to any one of SEQ ID NO: 154 through SEQ ID NO: 186. In yet other aspects of this embodiment, a binding domain has, e.g., at least one, two or three contiguous amino acid deletions relative to any one of SEQ ID NO: 154 through SEQ ID NO: 186. In yet other aspects of this embodiment, a binding domain has, e.g., at most one, two or three contiguous amino acid deletions relative to any one of SEQ ID NO: 154 through SEQ ID NO: 186. In still other aspects of this embodiment, a binding domain has, e.g., at least one, two or three contiguous amino acid additions relative to any one of SEQ ID NO: 154 through SEQ ID NO: 186. In yet other aspects of this embodiment, a binding domain has, e.g., at most one, two or three contiguous amino acid additions relative to any one of SEQ ID NO: 154 through SEQ ID NO: 186.

In an aspect of the invention, a modified Clostridial toxin comprises, in part, a Clostridial toxin enzymatic domain. As used herein, the term “Clostridial toxin enzymatic domain” means any Clostridial toxin polypeptide that can execute the enzymatic target modification step of the intoxication process. Thus, a Clostridial toxin enzymatic domain specifically targets and proteolytically cleavages of a Clostridial toxin substrate, such as, e.g., SNARE proteins like a SNAP-25 substrate, a VAMP substrate and a Syntaxin substrate. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain. Other non-limiting examples of a Clostridial toxin enzymatic domain include, e.g., amino acids 1-448 of SEQ ID NO: 134, amino acids 1-441 of SEQ ID NO: 135, amino acids 1-449 of SEQ ID NO: 136, amino acids 1-445 of SEQ ID NO: 137, amino acids 1-422 of SEQ ID NO: 138, amino acids 1-439 of SEQ ID NO: 139, amino acids 1-446 of SEQ ID NO: 140, amino acids 1-457 of SEQ ID NO: 141, amino acids 1-431 of SEQ ID NO: 142, and amino acids 1-422 of SEQ ID NO: 143.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimeras, active Clostridial toxin enzymatic domain fragments thereof, or any combination thereof.

As used herein, the term “Clostridial toxin enzymatic domain variant,” whether naturally-occurring or non-naturally-occurring, means a Clostridial toxin enzymatic domain that has at least one amino acid change from the corresponding region of the disclosed reference sequences (Table 1) and can be described in percent identity to the corresponding region of that reference sequence. Unless expressly indicated, Clostridial toxin enzymatic domain variants useful to practice disclosed embodiments are variants that execute the enzymatic target modification step of the intoxication process. As non-limiting examples, a BoNT/A enzymatic domain variant comprising amino acids 1-448 of SEQ ID NO: 134 will have at least one amino acid difference, such as, e.g., an amino acid substitution, deletion or addition, as compared to the amino acid region 1-448 of SEQ ID NO: 134; a BoNT/B enzymatic domain variant comprising amino acids 1-441 of SEQ ID NO: 135 will have at least one amino acid difference, such as, e.g., an amino acid substitution, deletion or addition, as compared to the amino acid region 1-441 of SEQ ID NO: 135; a BoNT/C1 enzymatic domain variant comprising amino acids 1-449 of SEQ ID NO: 136 will have at least one amino acid difference, such as, e.g., an amino acid substitution, deletion or addition, as compared to the amino acid region 1-449 of SEQ ID NO: 136; a BoNT/D enzymatic domain variant comprising amino acids 1-445 of SEQ ID NO: 137 will have at least one amino acid difference, such as, e.g., an amino acid substitution, deletion or addition, as compared to the amino acid region 1-445 of SEQ ID NO: 137; a BoNT/E enzymatic domain variant comprising amino acids 1-422 of SEQ ID NO: 138 will have at least one amino acid difference, such as, e.g., an amino acid substitution, deletion or addition, as compared to the amino acid region 1-422 of SEQ ID NO: 138; a BoNT/F enzymatic domain variant comprising amino acids 1-439 of SEQ ID NO: 139 will have at least one amino acid difference, such as, e.g., an amino acid substitution, deletion or addition, as compared to the amino acid region 1-439 of SEQ ID NO: 139; a BoNT/G enzymatic domain variant comprising amino acids 1-446 of SEQ ID NO: 140 will have at least one amino acid difference, such as, e.g., an amino acid substitution, deletion or addition, as compared to the amino acid region 1-446 of SEQ ID NO: 140; a TeNT enzymatic domain variant comprising amino acids 1-457 of SEQ ID NO: 141 will have at least one amino acid difference, such as, e.g., an amino acid substitution, deletion or addition, as compared to the amino acid region 1-457 of SEQ ID NO: 141; a BaNT enzymatic domain variant comprising amino acids 1-431 of SEQ ID NO: 142 will have at least one amino acid difference, such as, e.g., an amino acid substitution, deletion or addition, as compared to the amino acid region 1-431 of SEQ ID NO: 142; and a BuNT enzymatic domain variant comprising amino acids 1-422 of SEQ ID NO: 143 will have at least one amino acid difference, such as, e.g., an amino acid substitution, deletion or addition, as compared to the amino acid region 1-422 of SEQ ID NO: 143.

As used herein, the term “naturally occurring Clostridial toxin enzymatic domain variant” means any Clostridial toxin enzymatic domain produced by a naturally-occurring process, including, without limitation, Clostridial toxin enzymatic domain isoforms produced from alternatively-spliced transcripts, Clostridial toxin enzymatic domain isoforms produced by spontaneous mutation and Clostridial toxin enzymatic domain subtypes. A naturally occurring Clostridial toxin enzymatic domain variant can function in substantially the same manner as the reference Clostridial toxin enzymatic domain on which the naturally occurring Clostridial toxin enzymatic domain variant is based, and can be substituted for the reference Clostridial toxin enzymatic domain in any aspect of the present invention. A non-limiting example of a naturally occurring Clostridial toxin enzymatic domain variant is a Clostridial toxin enzymatic domain isoform such as, e.g., a BoNT/A enzymatic domain isoform, a BoNT/B enzymatic domain isoform, a BoNT/C1 enzymatic domain isoform, a BoNT/D enzymatic domain isoform, a BoNT/E enzymatic domain isoform, a BoNT/F enzymatic domain isoform, a BoNT/G enzymatic domain isoform, and a TeNT enzymatic domain isoform. Another non-limiting example of a naturally occurring Clostridial toxin enzymatic domain variant is a Clostridial toxin enzymatic domain subtype such as, e.g., an enzymatic domain from subtype BoNT/A1, BoNT/A2, BoNT/A3, BoNT/A4, and BoNT/A5; an enzymatic domain from subtype BoNT/B1, BoNT/B2, BoNT/B bivalent and BoNT/B nonproteolytic; an enzymatic domain from subtype BoNT/C1-1 and BoNT/C1-2; an enzymatic domain from subtype BoNT/E1, BoNT/E2 and BoNT/E3; and an enzymatic domain from subtype BoNT/F1, BoNT/F2, BoNT/F3 and BoNT/F4.

As used herein, the term “non-naturally occurring Clostridial toxin enzymatic domain variant” means any Clostridial toxin enzymatic domain produced with the aid of human manipulation, including, without limitation, Clostridial toxin enzymatic domains produced by genetic engineering using random mutagenesis or rational design and Clostridial toxin enzymatic domains produced by chemical synthesis. Non-limiting examples of non-naturally occurring Clostridial toxin enzymatic domain variants include, e.g., conservative Clostridial toxin enzymatic domain variants, non-conservative Clostridial toxin enzymatic domain variants, Clostridial toxin enzymatic domain chimeric variants and active Clostridial toxin enzymatic domain fragments. Other non-limiting examples of a non-naturally occurring Clostridial toxin enzymatic domain variant include, e.g., non-naturally occurring BoNT/A enzymatic domain variants, non-naturally occurring BoNT/B enzymatic domain variants, non-naturally occurring BoNT/C1 enzymatic domain variants, non-naturally occurring BoNT/D enzymatic domain variants, non-naturally occurring BoNT/E enzymatic domain variants, non-naturally occurring BoNT/F enzymatic domain variants, non-naturally occurring BoNT/G enzymatic domain variants, non-naturally occurring TeNT enzymatic domain variants, non-naturally occurring BaNT enzymatic domain variants, and non-naturally occurring BuNT enzymatic domain variants.

As used herein, the term “conservative Clostridial toxin enzymatic domain variant” means a Clostridial toxin enzymatic domain that has at least one amino acid substituted by another amino acid or an amino acid analog that has at least one property similar to that of the original amino acid from the reference Clostridial toxin enzymatic domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin enzymatic domain variant can function in substantially the same manner as the reference Clostridial toxin enzymatic domain on which the conservative Clostridial toxin enzymatic domain variant is based, and can be substituted for the reference Clostridial toxin enzymatic domain in any aspect of the present invention. Non-limiting examples of a conservative Clostridial toxin enzymatic domain variant include, e.g., conservative BoNT/A enzymatic domain variants, conservative BoNT/B enzymatic domain variants, conservative BoNT/C1 enzymatic domain variants, conservative BoNT/D enzymatic domain variants, conservative BoNT/E enzymatic domain variants, conservative BoNT/F enzymatic domain variants, conservative BoNT/G enzymatic domain variants, and conservative TeNT enzymatic domain variants, conservative BaNT enzymatic domain variants, and conservative BuNT enzymatic domain variants.

As used herein, the term “non-conservative Clostridial toxin enzymatic domain variant” means a Clostridial toxin enzymatic domain in which 1) at least one amino acid is deleted from the reference Clostridial toxin enzymatic domain on which the non-conservative Clostridial toxin enzymatic domain variant is based; 2) at least one amino acid added to the reference Clostridial toxin enzymatic domain on which the non-conservative Clostridial toxin enzymatic domain is based; or 3) at least one amino acid is substituted by another amino acid or an amino acid analog that does not share any property similar to that of the original amino acid from the reference Clostridial toxin enzymatic domain sequence (Table 1). A non-conservative Clostridial toxin enzymatic domain variant can function in substantially the same manner as the reference Clostridial toxin enzymatic domain on which the non-conservative Clostridial toxin enzymatic domain variant is based, and can be substituted for the reference Clostridial toxin enzymatic domain in any aspect of the present invention. Non-limiting examples of a non-conservative Clostridial toxin enzymatic domain variant include, e.g., non-conservative BoNT/A enzymatic domain variants, non-conservative BoNT/B enzymatic domain variants, non-conservative BoNT/C1 enzymatic domain variants, non-conservative BoNT/D enzymatic domain variants, non-conservative BoNT/E enzymatic domain variants, non-conservative BoNT/F enzymatic domain variants, non-conservative BoNT/G enzymatic domain variants, and non-conservative TeNT enzymatic domain variants, non-conservative BaNT enzymatic domain variants, and non-conservative BuNT enzymatic domain variants.

As used herein, the term “Clostridial toxin enzymatic domain chimeric” means a polypeptide comprising at least a portion of a Clostridial toxin enzymatic domain and at least a portion of at least one other polypeptide to form a toxin enzymatic domain with at least one property different from the reference Clostridial toxin enzymatic domains of Table 1, with the proviso that this Clostridial toxin enzymatic domain chimeric is still capable of specifically targeting the core components of the neurotransmitter release apparatus and thus participate in executing the overall cellular mechanism whereby a Clostridial toxin proteolytically cleaves a substrate. Such Clostridial toxin enzymatic domain chimerics are described in, e.g., Lance E. Steward et al., Leucine-based Motif and Clostridial Toxins, U.S. Patent Publication 2003/0027752 (Feb. 6, 2003); Lance E. Steward et al., Clostridial Neurotoxin Compositions and Modified Clostridial Neurotoxins, U.S. Patent Publication 2003/0219462 (Nov. 27, 2003); and Lance E. Steward et al., Clostridial Neurotoxin Compositions and Modified Clostridial Neurotoxins, U.S. Patent Publication 2004/0220386 (Nov. 4, 2004), each of which is hereby incorporated by reference in its entirety. Non-limiting examples of a Clostridial toxin enzymatic domain chimeric include, e.g., BoNT/A enzymatic domain chimerics, BoNT/B enzymatic domain chimerics, BoNT/C1 enzymatic domain chimerics, BoNT/D enzymatic domain chimerics, BoNT/E enzymatic domain chimerics, BoNT/F enzymatic domain chimerics, BoNT/G enzymatic domain chimerics, and TeNT enzymatic domain chimerics, BaNT enzymatic domain chimerics, and BuNT enzymatic domain chimerics.

As used herein, the term “active Clostridial toxin enzymatic domain fragment” means any of a variety of Clostridial toxin fragments comprising the enzymatic domain can be useful in aspects of the present invention with the proviso that these enzymatic domain fragments can specifically target the core components of the neurotransmitter release apparatus and thus participate in executing the overall cellular mechanism whereby a Clostridial toxin proteolytically cleaves a substrate. The enzymatic domains of Clostridial toxins are approximately 420-460 amino acids in length and comprise an enzymatic domain (Table 1). Research has shown that the entire length of a Clostridial toxin enzymatic domain is not necessary for the enzymatic activity of the enzymatic domain. As a non-limiting example, the first eight amino acids of the BoNT/A enzymatic domain (residues 1-8 of SEQ ID NO: 134) are not required for enzymatic activity. As another non-limiting example, the first eight amino acids of the TeNT enzymatic domain (residues 1-8 of SEQ ID NO: 141) are not required for enzymatic activity. Likewise, the carboxyl-terminus of the enzymatic domain is not necessary for activity. As a non-limiting example, the last 32 amino acids of the BoNT/A enzymatic domain (residues 417-448 of SEQ ID NO: 134) are not required for enzymatic activity. As another non-limiting example, the last 31 amino acids of the TeNT enzymatic domain (residues 427-457 of SEQ ID NO: 141) are not required for enzymatic activity. Thus, aspects of this embodiment can include Clostridial toxin enzymatic domains comprising an enzymatic domain having a length of, e.g., at least 350 amino acids, at least 375 amino acids, at least 400 amino acids, at least 425 amino acids and at least 450 amino acids. Other aspects of this embodiment can include Clostridial toxin enzymatic domains comprising an enzymatic domain having a length of, e.g., at most 350 amino acids, at most 375 amino acids, at most 400 amino acids, at most 425 amino acids and at most 450 amino acids.

Thus, in an embodiment, a Clostridial toxin enzymatic domain comprises a naturally occurring Clostridial toxin enzymatic domain variant. In an aspect of this embodiment, a naturally occurring Clostridial toxin enzymatic domain variant is a naturally occurring BoNT/A enzymatic domain variant, such as, e.g., an enzymatic domain from a BoNT/A isoform or an enzymatic domain from a BoNT/A subtype; a naturally occurring BoNT/B enzymatic domain variant, such as, e.g., an enzymatic domain from a BoNT/B isoform or an enzymatic domain from a BoNT/B subtype; a naturally occurring BoNT/C1 enzymatic domain variant, such as, e.g., an enzymatic domain from a BoNT/C1 isoform or an enzymatic domain from a BoNT/C1 subtype; a naturally occurring BoNT/D enzymatic domain variant, such as, e.g., an enzymatic domain from a BoNT/D isoform or an enzymatic domain from a BoNT/D subtype; a naturally occurring BoNT/E enzymatic domain variant, such as, e.g., an enzymatic domain from a BoNT/E isoform or an enzymatic domain from a BoNT/E subtype; a naturally occurring BoNT/F enzymatic domain variant, such as, e.g., an enzymatic domain from a BoNT/F isoform or an enzymatic domain from a BoNT/F subtype; a naturally occurring BoNT/G enzymatic domain variant, such as, e.g., an enzymatic domain from a BoNT/G isoform or an enzymatic domain from a BoNT/G subtype; a naturally occurring TeNT enzymatic domain variant, such as, e.g., an enzymatic domain from a TeNT isoform or an enzymatic domain from a TeNT subtype; a naturally occurring BaNT enzymatic domain variant, such as, e.g., an enzymatic domain from a BaNT isoform or an enzymatic domain from a BaNT subtype; or a naturally occurring BuNT enzymatic domain variant, such as, e.g., an enzymatic domain from a BuNT isoform or an enzymatic domain from a BuNT subtype.

In aspects of this embodiment, a naturally occurring Clostridial toxin enzymatic domain variant is a polypeptide having an amino acid identity to the reference Clostridial toxin enzymatic domain on which the naturally occurring Clostridial toxin enzymatic domain variant is based of, e.g., at least 70%, at least 75%, at least 80%, at least 85%, at least 90% or at least 95%. In yet other aspects of this embodiment, a naturally occurring Clostridial toxin enzymatic domain variant is a polypeptide having an amino acid identity to the reference Clostridial toxin enzymatic domain on which the naturally occurring Clostridial toxin enzymatic domain variant is based of, e.g., at most 70%, at most 75%, at most 80%, at most 85%, at most 90% or at most 95%.

In other aspects of this embodiment, a naturally occurring Clostridial toxin enzymatic domain variant is a polypeptide having, e.g., at most 1, 2, 3, 4, 5, 6, 7, 8, 9, 10, 20, 30, 40, 50, or 100 non-contiguous amino acid substitutions relative to the reference Clostridial toxin enzymatic domain on which the naturally occurring Clostridial toxin enzymatic domain variant is based; at least 1, 2, 3, 4, 5, 6, 7, 8, 9, 10, 20, 30, 40, 50, or 100 non-contiguous amino acid substitutions relative to the reference Clostridial toxin enzymatic domain on which the naturally occurring Clostridial toxin enzymatic domain variant is based; at most 1, 2, 3, 4, 5, 6, 7, 8, 9, 10, 20, 30, 40, 50, or 100 non-contiguous amino acid deletions relative to the reference Clostridial toxin enzymatic domain on which the naturally occurring Clostridial toxin enzymatic domain variant is based; at least 1, 2, 3, 4, 5, 6, 7, 8, 9, 10, 20, 30, 40, 50, or 100 non-contiguous amino acid deletions relative to the reference Clostridial toxin enzymatic domain on which the naturally occurring Clostridial toxin enzymatic domain variant is based; at most 1, 2, 3, 4, 5, 6, 7, 8, 9, 10, 20, 30, 40, 50, or 100 non-contiguous amino acid additions relative to the reference Clostridial toxin enzymatic domain on which the naturally occurring Clostridial toxin enzymatic domain variant is based; or at least 1, 2, 3, 4, 5, 6, 7, 8, 9, 10, 20, 30, 40, 50, or 100 non-contiguous amino acid additions relative to the reference Clostridial toxin enzymatic domain on which the naturally occurring Clostridial toxin enzymatic domain variant is based.

In yet other aspects of this embodiment, a naturally occurring Clostridial toxin enzymatic domain variant is a polypeptide having, e.g., at most 1, 2, 3, 4, 5, 6, 7, 8, 9, 10, 20, 30, 40, 50, or 100 contiguous amino acid substitutions relative to the reference Clostridial toxin enzymatic domain on which the naturally occurring Clostridial toxin enzymatic domain variant is based; at least 1, 2, 3, 4, 5, 6, 7, 8, 9, 10, 20, 30, 40, 50, or 100 contiguous amino acid substitutions relative to the reference Clostridial toxin enzymatic domain on which the naturally occurring Clostridial toxin enzymatic domain variant is based; at most 1, 2, 3, 4, 5, 6, 7, 8, 9, 10, 20, 30, 40, 50, or 100 contiguous amino acid deletions relative to the reference Clostridial toxin enzymatic domain on which the naturally occurring Clostridial toxin enzymatic domain variant is based; at least 1, 2, 3, 4, 5, 6, 7, 8, 9, 10, 20, 30, 40, 50, or 100 contiguous amino acid deletions relative to the reference Clostridial toxin enzymatic domain on which the naturally occurring Clostridial toxin enzymatic domain variant is based; at most 1, 2, 3, 4, 5, 6, 7, 8, 9, 10, 20, 30, 40, 50, or 100 contiguous amino acid additions relative to the reference Clostridial toxin enzymatic domain on which the naturally occurring Clostridial toxin enzymatic domain variant is based; or at least 1, 2, 3, 4, 5, 6, 7, 8, 9, 10, 20, 30, 40, 50, or 100 contiguous amino acid additions relative to the reference Clostridial toxin enzymatic domain on which the naturally occurring Clostridial toxin enzymatic domain variant is based.

In another embodiment, a Clostridial toxin enzymatic domain comprises a non-naturally occurring Clostridial toxin enzymatic domain variant. In an aspect of this embodiment, a non-naturally occurring Clostridial toxin enzymatic domain variant is a non-naturally occurring BoNT/A enzymatic domain variant, such as, e.g., a conservative BoNT/A enzymatic domain variant, a non-conservative BoNT/A enzymatic domain variant, a BoNT/A chimeric enzymatic domain, or an active BoNT/A enzymatic domain fragment; a non-naturally occurring BoNT/B enzymatic domain variant, such as, e.g., a conservative BoNT/B enzymatic domain variant, a non-conservative BoNT/B enzymatic domain variant, a BoNT/B chimeric enzymatic domain, or an active BoNT/B enzymatic domain fragment; a non-naturally occurring BoNT/C1 enzymatic domain variant, such as, e.g., a conservative BoNT/C1 enzymatic domain variant, a non-conservative BoNT/C1 enzymatic domain variant, a BoNT/C1 chimeric enzymatic domain, or an active BoNT/C1 enzymatic domain fragment; a non-naturally occurring BoNT/D enzymatic domain variant, such as, e.g., a conservative BoNT/D enzymatic domain variant, a non-conservative BoNT/D enzymatic domain variant, a BoNT/D chimeric enzymatic domain, or an active BoNT/D enzymatic domain fragment; a non-naturally occurring BoNT/E enzymatic domain variant, such as, e.g., a conservative BoNT/E enzymatic domain variant, a non-conservative BoNT/E enzymatic domain variant, a BoNT/E chimeric enzymatic domain, or an active BoNT/E enzymatic domain fragment; a non-naturally occurring BoNT/F enzymatic domain variant, such as, e.g., a conservative BoNT/F enzymatic domain variant, a non-conservative BoNT/F enzymatic domain variant, a BoNT/F chimeric enzymatic domain, or an active BoNT/F enzymatic domain fragment; a non-naturally occurring BoNT/G enzymatic domain variant, such as, e.g., a conservative BoNT/G enzymatic domain variant, a non-conservative BoNT/G enzymatic domain variant, a BoNT/G chimeric enzymatic domain, or an active BoNT/G enzymatic domain fragment; a non-naturally occurring TeNT enzymatic domain variant, such as, e.g., a conservative TeNT enzymatic domain variant, a non-conservative TeNT enzymatic domain variant, a TeNT chimeric enzymatic domain, or an active TeNT enzymatic domain fragment; a non-naturally occurring BaNT enzymatic domain variant, such as, e.g., a conservative BaNT enzymatic domain variant, a non-conservative BaNT enzymatic domain variant, a BaNT chimeric enzymatic domain, or an active BaNT enzymatic domain fragment; or a non-naturally occurring BuNT enzymatic domain variant, such as, e.g., a conservative BuNT enzymatic domain variant, a non-conservative BuNT enzymatic domain variant, a BuNT chimeric enzymatic domain, or an active BuNT enzymatic domain fragment.

In aspects of this embodiment, a non-naturally occurring Clostridial toxin enzymatic domain variant is a polypeptide having an amino acid identity to the reference Clostridial toxin enzymatic domain on which the non-naturally occurring Clostridial toxin enzymatic domain variant is based of, e.g., at least 70%, at least 75%, at least 80%, at least 85%, at least 90% or at least 95%. In yet other aspects of this embodiment, a non-naturally occurring Clostridial toxin enzymatic domain variant is a polypeptide having an amino acid identity to the reference Clostridial toxin enzymatic domain on which the non-naturally occurring Clostridial toxin enzymatic domain variant is based of, e.g., at most 70%, at most 75%, at most 80%, at most 85%, at most 90% or at most 95%.

In other aspects of this embodiment, a non-naturally occurring Clostridial toxin enzymatic domain variant is a polypeptide having, e.g., at most 1, 2, 3, 4, 5, 6, 7, 8, 9, 10, 20, 30, 40, 50, or 100 non-contiguous amino acid substitutions relative to the reference Clostridial toxin enzymatic domain on which the non-naturally occurring Clostridial toxin enzymatic domain variant is based; at least 1, 2, 3, 4, 5, 6, 7, 8, 9, 10, 20, 30, 40, 50, or 100 non-contiguous amino acid substitutions relative to the reference Clostridial toxin enzymatic domain on which the non-naturally occurring Clostridial toxin enzymatic domain variant is based; at most 1, 2, 3, 4, 5, 6, 7, 8, 9, 10, 20, 30, 40, 50, or 100 non-contiguous amino acid deletions relative to the reference Clostridial toxin enzymatic domain on which the non-naturally occurring Clostridial toxin enzymatic domain variant is based; at least 1, 2, 3, 4, 5, 6, 7, 8, 9, 10, 20, 30, 40, 50, or 100 non-contiguous amino acid deletions relative to the reference Clostridial toxin enzymatic domain on which the non-naturally occurring Clostridial toxin enzymatic domain variant is based; at most 1, 2, 3, 4, 5, 6, 7, 8, 9, 10, 20, 30, 40, 50, or 100 non-contiguous amino acid additions relative to the reference Clostridial toxin enzymatic domain on which the non-naturally occurring Clostridial toxin enzymatic domain variant is based; or at least 1, 2, 3, 4, 5, 6, 7, 8, 9, 10, 20, 30, 40, 50, or 100 non-contiguous amino acid additions relative to the reference Clostridial toxin enzymatic domain on which the non-naturally occurring Clostridial toxin enzymatic domain variant is based.

In yet other aspects of this embodiment, a non-naturally occurring Clostridial toxin enzymatic domain variant is a polypeptide having, e.g., at most 1, 2, 3, 4, 5, 6, 7, 8, 9, 10, 20, 30, 40, 50, or 100 contiguous amino acid substitutions relative to the reference Clostridial toxin enzymatic domain on which the non-naturally occurring Clostridial toxin enzymatic domain variant is based; at least 1, 2, 3, 4, 5, 6, 7, 8, 9, 10, 20, 30, 40, 50, or 100 contiguous amino acid substitutions relative to the reference Clostridial toxin enzymatic domain on which the non-naturally occurring Clostridial toxin enzymatic domain variant is based; at most 1, 2, 3, 4, 5, 6, 7, 8, 9, 10, 20, 30, 40, 50, or 100 contiguous amino acid deletions relative to the reference Clostridial toxin enzymatic domain on which the non-naturally occurring Clostridial toxin enzymatic domain variant is based; at least 1, 2, 3, 4, 5, 6, 7, 8, 9, 10, 20, 30, 40, 50, or 100 contiguous amino acid deletions relative to the reference Clostridial toxin enzymatic domain on which the non-naturally occurring Clostridial toxin enzymatic domain variant is based; at most 1, 2, 3, 4, 5, 6, 7, 8, 9, 10, 20, 30, 40, 50, or 100 contiguous amino acid additions relative to the reference Clostridial toxin enzymatic domain on which the non-naturally occurring Clostridial toxin enzymatic domain variant is based; or at least 1, 2, 3, 4, 5, 6, 7, 8, 9, 10, 20, 30, 40, 50, or 100 contiguous amino acid additions relative to the reference Clostridial toxin enzymatic domain on which the non-naturally occurring Clostridial toxin enzymatic domain variant is based.

In another embodiment, a hydrophic amino acid at one particular position in the polypeptide chain of the Clostridial toxin enzymatic domain variant can be substituted with another hydrophic amino acid. Examples of hydrophic amino acids include, e.g., C, F, I, L, M, V and W. In another aspect of this embodiment, an aliphatic amino acid at one particular position in the polypeptide chain of the Clostridial toxin enzymatic domain variant can be substituted with another aliphatic amino acid. Examples of aliphatic amino acids include, e.g., A, I, L, P, and V. In yet another aspect of this embodiment, an aromatic amino acid at one particular position in the polypeptide chain of the Clostridial toxin enzymatic domain variant can be substituted with another aromatic amino acid. Examples of aromatic amino acids include, e.g., F, H, W and Y. In still another aspect of this embodiment, a stacking amino acid at one particular position in the polypeptide chain of the Clostridial toxin enzymatic domain variant can be substituted with another stacking amino acid. Examples of stacking amino acids include, e.g., F, H, W and Y. In a further aspect of this embodiment, a polar amino acid at one particular position in the polypeptide chain of the Clostridial toxin enzymatic domain variant can be substituted with another polar amino acid. Examples of polar amino acids include, e.g., D, E, K, N, Q, and R. In a further aspect of this embodiment, a less polar or indifferent amino acid at one particular position in the polypeptide chain of the Clostridial toxin enzymatic domain variant can be substituted with another less polar or indifferent amino acid. Examples of less polar or indifferent amino acids include, e.g., A, H, G, P, S, T, and Y. In a yet further aspect of this embodiment, a positive charged amino acid at one particular position in the polypeptide chain of the Clostridial toxin enzymatic domain variant can be substituted with another positive charged amino acid. Examples of positive charged amino acids include, e.g., K, R, and H. In a still further aspect of this embodiment, a negative charged amino acid at one particular position in the polypeptide chain of the Clostridial toxin enzymatic domain variant can be substituted with another negative charged amino acid. Examples of negative charged amino acids include, e.g., D and E. In another aspect of this embodiment, a small amino acid at one particular position in the polypeptide chain of the Clostridial toxin enzymatic domain variant can be substituted with another small amino acid. Examples of small amino acids include, e.g., A, D, G, N, P, S, and T. In yet another aspect of this embodiment, a C-beta branching amino acid at one particular position in the polypeptide chain of the Clostridial toxin enzymatic domain variant can be substituted with another C-beta branching amino acid. Examples of C-beta branching amino acids include, e.g., I, T and V.

In another aspect of the invention, a modified Clostridial toxin comprises, in part, a Clostridial toxin translocation domain. As used herein, the term “Clostridial toxin translocation domain” means any Clostridial toxin polypeptide that can execute the translocation step of the intoxication process that mediates Clostridial toxin light chain translocation. By “translocation” is meant the ability to facilitate the transport of a polypeptide through a vesicular membrane, thereby exposing some or all of the polypeptide to the cytoplasm. In the various botulinum neurotoxins translocation is thought to involve an allosteric conformational change of the heavy chain caused by a decrease in pH within the endosome. This conformational change appears to involve and be mediated by the N terminal half of the heavy chain and to result in the formation of pores in the vesicular membrane; this change permits the movement of the proteolytic light chain from within the endosomal vesicle into the cytoplasm. See e.g., Lacy, et al., Nature Struct. Biol. 5:898-902 (October 1998). Thus, a Clostridial toxin translocation domain facilitates the movement of a Clostridial toxin light chain across a membrane of an intracellular vesicle into the cytoplasm of a cell. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain. Other non-limiting examples of a Clostridial toxin translocation domain include, e.g., amino acids 449-873 of SEQ ID NO: 134, amino acids 442-860 of SEQ ID NO: 135, amino acids 450-868 of SEQ ID NO: 136, amino acids 446-864 of SEQ ID NO: 137, amino acids 423-847 of SEQ ID NO: 138, amino acids 440-866 of SEQ ID NO: 139, amino acids 447-865 of SEQ ID NO: 140, amino acids 458-881 of SEQ ID NO: 141, amino acids 432-857 of SEQ ID NO: 142, and amino acids 423-847 of SEQ ID NO: 143.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof.

As used herein, the term “Clostridial toxin translocation domain variant,” whether naturally-occurring or non-naturally-occurring, means a Clostridial toxin translocation domain that has at least one amino acid change from the corresponding region of the disclosed reference sequences (Table 1) and can be described in percent identity to the corresponding region of that reference sequence. Unless expressly indicated, Clostridial toxin translocation domain variants useful to practice disclosed embodiments are variants that execute the translocation step of the intoxication process that mediates Clostridial toxin light chain translocation. As non-limiting examples, a BoNT/A translocation domain variant comprising amino acids 449-873 of SEQ ID NO: 134 will have at least one amino acid difference, such as, e.g., an amino acid substitution, deletion or addition, as compared to the amino acid region 449-873 of SEQ ID NO: 134; a BoNT/B translocation domain variant comprising amino acids 442-860 of SEQ ID NO: 135 will have at least one amino acid difference, such as, e.g., an amino acid substitution, deletion or addition, as compared to the amino acid region 442-860 of SEQ ID NO: 135; a BoNT/C1 translocation domain variant comprising amino acids 450-868 of SEQ ID NO: 136 will have at least one amino acid difference, such as, e.g., an amino acid substitution, deletion or addition, as compared to the amino acid region 450-868 of SEQ ID NO: 136; a BoNT/D translocation domain variant comprising amino acids 446-864 of SEQ ID NO: 137 will have at least one amino acid difference, such as, e.g., an amino acid substitution, deletion or addition, as compared to the amino acid region 446-864 of SEQ ID NO: 137; a BoNT/E translocation domain variant comprising amino acids 423-847 of SEQ ID NO: 138 will have at least one amino acid difference, such as, e.g., an amino acid substitution, deletion or addition, as compared to the amino acid region 423-847 of SEQ ID NO: 138; a BoNT/F translocation domain variant comprising amino acids 440-866 of SEQ ID NO: 139 will have at least one amino acid difference, such as, e.g., an amino acid substitution, deletion or addition, as compared to the amino acid region 440-866 of SEQ ID NO: 139; a BoNT/G translocation domain variant comprising amino acids 447-865 of SEQ ID NO: 140 will have at least one amino acid difference, such as, e.g., an amino acid substitution, deletion or addition, as compared to the amino acid region 447-865 of SEQ ID NO: 140; a TeNT translocation domain variant comprising amino acids 458-881 of SEQ ID NO: 141 will have at least one amino acid difference, such as, e.g., an amino acid substitution, deletion or addition, as compared to the amino acid region 458-881 of SEQ ID NO: 141; a BaNT translocation domain variant comprising amino acids 432-857 of SEQ ID NO: 142 will have at least one amino acid difference, such as, e.g., an amino acid substitution, deletion or addition, as compared to the amino acid region 432-857 of SEQ ID NO: 142; and a BuNT translocation domain variant comprising amino acids 423-847 of SEQ ID NO: 143 will have at least one amino acid difference, such as, e.g., an amino acid substitution, deletion or addition, as compared to the amino acid region 423-847 of SEQ ID NO: 143.

As used herein, the term “naturally occurring Clostridial toxin translocation domain variant” means any Clostridial toxin translocation domain produced by a naturally-occurring process, including, without limitation, Clostridial toxin translocation domain isoforms produced from alternatively-spliced transcripts, Clostridial toxin translocation domain isoforms produced by spontaneous mutation and Clostridial toxin translocation domain subtypes. A naturally occurring Clostridial toxin translocation domain variant can function in substantially the same manner as the reference Clostridial toxin translocation domain on which the naturally occurring Clostridial toxin translocation domain variant is based, and can be substituted for the reference Clostridial toxin translocation domain in any aspect of the present invention. A non-limiting example of a naturally occurring Clostridial toxin translocation domain variant is a Clostridial toxin translocation domain isoform such as, e.g., a BoNT/A translocation domain isoform, a BoNT/B translocation domain isoform, a BoNT/C1 translocation domain isoform, a BoNT/D translocation domain isoform, a BoNT/E translocation domain isoform, a BoNT/F translocation domain isoform, a BoNT/G translocation domain isoform, a TeNT translocation domain isoform, a BaNT translocation domain isoform, and a BuNT translocation domain isoform. Another non-limiting example of a naturally occurring Clostridial toxin translocation domain variant is a Clostridial toxin translocation domain subtype such as, e.g., a translocation domain from subtype BoNT/A1, BoNT/A2, BoNT/A3, BoNT/A4, and BoNT/A5; a translocation domain from subtype BoNT/B1, BoNT/B2, BoNT/B bivalent and BoNT/B nonproteolytic; a translocation domain from subtype BoNT/C1-1 and BoNT/C1-2; a translocation domain from subtype BoNT/E1, BoNT/E2 and BoNT/E3; and a translocation domain from subtype BoNT/F1, BoNT/F2, BoNT/F3 and BoNT/F4.

As used herein, the term “non-naturally occurring Clostridial toxin translocation domain variant” means any Clostridial toxin translocation domain produced with the aid of human manipulation, including, without limitation, Clostridial toxin translocation domains produced by genetic engineering using random mutagenesis or rational design and Clostridial toxin translocation domains produced by chemical synthesis. Non-limiting examples of non-naturally occurring Clostridial toxin translocation domain variants include, e.g., conservative Clostridial toxin translocation domain variants, non-conservative Clostridial toxin translocation domain variants, Clostridial toxin translocation domain chimeric variants and active Clostridial toxin translocation domain fragments. Non-limiting examples of a non-naturally occurring Clostridial toxin translocation domain variant include, e.g., non-naturally occurring BoNT/A translocation domain variants, non-naturally occurring BoNT/B translocation domain variants, non-naturally occurring BoNT/C1 translocation domain variants, non-naturally occurring BoNT/D translocation domain variants, non-naturally occurring BoNT/E translocation domain variants, non-naturally occurring BoNT/F translocation domain variants, non-naturally occurring BoNT/G translocation domain variants, non-naturally occurring TeNT translocation domain variants, non-naturally occurring BaNT translocation domain variants, and non-naturally occurring BuNT translocation domain variants.

As used herein, the term “conservative Clostridial toxin translocation domain variant” means a Clostridial toxin translocation domain that has at least one amino acid substituted by another amino acid or an amino acid analog that has at least one property similar to that of the original amino acid from the reference Clostridial toxin translocation domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin translocation domain variant can function in substantially the same manner as the reference Clostridial toxin translocation domain on which the conservative Clostridial toxin translocation domain variant is based, and can be substituted for the reference Clostridial toxin translocation domain in any aspect of the present invention. Non-limiting examples of a conservative Clostridial toxin translocation domain variant include, e.g., conservative BoNT/A translocation domain variants, conservative BoNT/B translocation domain variants, conservative BoNT/C1 translocation domain variants, conservative BoNT/D translocation domain variants, conservative BoNT/E translocation domain variants, conservative BoNT/F translocation domain variants, conservative BoNT/G translocation domain variants, conservative TeNT translocation domain variants, conservative BaNT translocation domain variants, and conservative BuNT translocation domain variants.

As used herein, the term “non-conservative Clostridial toxin translocation domain variant” means a Clostridial toxin translocation domain in which 1) at least one amino acid is deleted from the reference Clostridial toxin translocation domain on which the non-conservative Clostridial toxin translocation domain variant is based; 2) at least one amino acid added to the reference Clostridial toxin translocation domain on which the non-conservative Clostridial toxin translocation domain is based; or 3) at least one amino acid is substituted by another amino acid or an amino acid analog that does not share any property similar to that of the original amino acid from the reference Clostridial toxin translocation domain sequence (Table 1). A non-conservative Clostridial toxin translocation domain variant can function in substantially the same manner as the reference Clostridial toxin translocation domain on which the non-conservative Clostridial toxin translocation domain variant is based, and can be substituted for the reference Clostridial toxin translocation domain in any aspect of the present invention. Non-limiting examples of a non-conservative Clostridial toxin translocation domain variant include, e.g., non-conservative BoNT/A translocation domain variants, non-conservative BoNT/B translocation domain variants, non-conservative BoNT/C1 translocation domain variants, non-conservative BoNT/D translocation domain variants, non-conservative BoNT/E translocation domain variants, non-conservative BoNT/F translocation domain variants, non-conservative BoNT/G translocation domain variants, and non-conservative TeNT translocation domain variants, non-conservative BaNT translocation domain variants, and non-conservative BuNT translocation domain variants.

As used herein, the term “Clostridial toxin translocation domain chimeric” means a polypeptide comprising at least a portion of a Clostridial toxin translocation domain and at least a portion of at least one other polypeptide to form a toxin translocation domain with at least one property different from the reference Clostridial toxin translocation domains of Table 1, with the proviso that this Clostridial toxin translocation domain chimeric is still capable of specifically targeting the core components of the neurotransmitter release apparatus and thus participate in executing the overall cellular mechanism whereby a Clostridial toxin proteolytically cleaves a substrate. Non-limiting examples of a Clostridial toxin translocation domain chimeric include, e.g., BoNT/A translocation domain chimerics, BoNT/B translocation domain chimerics, BoNT/C1 translocation domain chimerics, BoNT/D translocation domain chimerics, BoNT/E translocation domain chimerics, BoNT/F translocation domain chimerics, BoNT/G translocation domain chimerics, and TeNT translocation domain chimerics, BaNT translocation domain chimerics, and BuNT translocation domain chimerics.

As used herein, the term “active Clostridial toxin translocation domain fragment” means any of a variety of Clostridial toxin fragments comprising the translocation domain can be useful in aspects of the present invention with the proviso that these active fragments can facilitate the release of the LC from intracellular vesicles into the cytoplasm of the target cell and thus participate in executing the overall cellular mechanism whereby a Clostridial toxin proteolytically cleaves a substrate. The translocation domains from the heavy chains of Clostridial toxins are approximately 410-430 amino acids in length and comprise a translocation domain (Table 1). Research has shown that the entire length of a translocation domain from a Clostridial toxin heavy chain is not necessary for the translocating activity of the translocation domain. Thus, aspects of this embodiment can include Clostridial toxin translocation domains comprising a translocation domain having a length of, e.g., at least 350 amino acids, at least 375 amino acids, at least 400 amino acids and at least 425 amino acids. Other aspects of this embodiment can include Clostridial toxin translocation domains comprising translocation domain having a length of, e.g., at most 350 amino acids, at most 375 amino acids, at most 400 amino acids and at most 425 amino acids.

Thus, in an embodiment, a Clostridial toxin translocation domain comprises a naturally occurring Clostridial toxin translocation domain variant. In an aspect of this embodiment, a naturally occurring Clostridial toxin translocation domain variant is a naturally occurring BoNT/A translocation domain variant, such as, e.g., an translocation domain from a BoNT/A isoform or an translocation domain from a BoNT/A subtype; a naturally occurring BoNT/B translocation domain variant, such as, e.g., an translocation domain from a BoNT/B isoform or an translocation domain from a BoNT/B subtype; a naturally occurring BoNT/C1 translocation domain variant, such as, e.g., an translocation domain from a BoNT/C1 isoform or an translocation domain from a BoNT/C1 subtype; a naturally occurring BoNT/D translocation domain variant, such as, e.g., an translocation domain from a BoNT/D isoform or an translocation domain from a BoNT/D subtype; a naturally occurring BoNT/E translocation domain variant, such as, e.g., an translocation domain from a BoNT/E isoform or an translocation domain from a BoNT/E subtype; a naturally occurring BoNT/F translocation domain variant, such as, e.g., an translocation domain from a BoNT/F isoform or an translocation domain from a BoNT/F subtype; a naturally occurring BoNT/G translocation domain variant, such as, e.g., an translocation domain from a BoNT/G isoform or an translocation domain from a BoNT/G subtype; a naturally occurring TeNT translocation domain variant, such as, e.g., an translocation domain from a TeNT isoform or an translocation domain from a TeNT subtype; a naturally occurring BaNT translocation domain variant, such as, e.g., an translocation domain from a BaNT isoform or an translocation domain from a BaNT subtype; or a naturally occurring BuNT translocation domain variant, such as, e.g., an translocation domain from a BuNT isoform or an translocation domain from a BuNT subtype.

In aspects of this embodiment, a naturally occurring Clostridial toxin translocation domain variant is a polypeptide having an amino acid identity to the reference Clostridial toxin translocation domain on which the naturally occurring Clostridial toxin translocation domain variant is based of, e.g., at least 70%, at least 75%, at least 80%, at least 85%, at least 90% or at least 95%. In yet other aspects of this embodiment, a naturally occurring Clostridial toxin translocation domain variant is a polypeptide having an amino acid identity to the reference Clostridial toxin translocation domain on which the naturally occurring Clostridial toxin translocation domain variant is based of, e.g., at most 70%, at most 75%, at most 80%, at most 85%, at most 90% or at most 95%.

In other aspects of this embodiment, a naturally occurring Clostridial toxin translocation domain variant is a polypeptide having, e.g., at most 1, 2, 3, 4, 5, 6, 7, 8, 9, 10, 20, 30, 40, 50, or 100 non-contiguous amino acid substitutions relative to the reference Clostridial toxin translocation domain on which the naturally occurring Clostridial toxin translocation domain variant is based; at least 1, 2, 3, 4, 5, 6, 7, 8, 9, 10, 20, 30, 40, 50, or 100 non-contiguous amino acid substitutions relative to the reference Clostridial toxin translocation domain on which the naturally occurring Clostridial toxin translocation domain variant is based; at most 1, 2, 3, 4, 5, 6, 7, 8, 9, 10, 20, 30, 40, 50, or 100 non-contiguous amino acid deletions relative to the reference Clostridial toxin translocation domain on which the naturally occurring Clostridial toxin translocation domain variant is based; at least 1, 2, 3, 4, 5, 6, 7, 8, 9, 10, 20, 30, 40, 50, or 100 non-contiguous amino acid deletions relative to the reference Clostridial toxin translocation domain on which the naturally occurring Clostridial toxin translocation domain variant is based; at most 1, 2, 3, 4, 5, 6, 7, 8, 9, 10, 20, 30, 40, 50, or 100 non-contiguous amino acid additions relative to the reference Clostridial toxin translocation domain on which the naturally occurring Clostridial toxin translocation domain variant is based; or at least 1, 2, 3, 4, 5, 6, 7, 8, 9, 10, 20, 30, 40, 50, or 100 non-contiguous amino acid additions relative to the reference Clostridial toxin translocation domain on which the naturally occurring Clostridial toxin translocation domain variant is based.

In yet other aspects of this embodiment, a naturally occurring Clostridial toxin translocation domain variant is a polypeptide having, e.g., at most 1, 2, 3, 4, 5, 6, 7, 8, 9, 10, 20, 30, 40, 50, or 100 contiguous amino acid substitutions relative to the reference Clostridial toxin translocation domain on which the naturally occurring Clostridial toxin translocation domain variant is based; at least 1, 2, 3, 4, 5, 6, 7, 8, 9, 10, 20, 30, 40, 50, or 100 contiguous amino acid substitutions relative to the reference Clostridial toxin translocation domain on which the naturally occurring Clostridial toxin translocation domain variant is based; at most 1, 2, 3, 4, 5, 6, 7, 8, 9, 10, 20, 30, 40, 50, or 100 contiguous amino acid deletions relative to the reference Clostridial toxin translocation domain on which the naturally occurring Clostridial toxin translocation domain variant is based; at least 1, 2, 3, 4, 5, 6, 7, 8, 9, 10, 20, 30, 40, 50, or 100 contiguous amino acid deletions relative to the reference Clostridial toxin translocation domain on which the naturally occurring Clostridial toxin translocation domain variant is based; at most 1, 2, 3, 4, 5, 6, 7, 8, 9, 10, 20, 30, 40, 50, or 100 contiguous amino acid additions relative to the reference Clostridial toxin translocation domain on which the naturally occurring Clostridial toxin translocation domain variant is based; or at least 1, 2, 3, 4, 5, 6, 7, 8, 9, 10, 20, 30, 40, 50, or 100 contiguous amino acid additions relative to the reference Clostridial toxin translocation domain on which the naturally occurring Clostridial toxin translocation domain variant is based.

In another embodiment, a Clostridial toxin translocation domain comprises a non-naturally occurring Clostridial toxin translocation domain variant. In an aspect of this embodiment, a non-naturally occurring Clostridial toxin translocation domain variant is a non-naturally occurring BoNT/A translocation domain variant, such as, e.g., a conservative BoNT/A translocation domain variant, a non-conservative BoNT/A translocation domain variant, a BoNT/A chimeric translocation domain, or an active BoNT/A translocation domain fragment; a non-naturally occurring BoNT/B translocation domain variant, such as, e.g., a conservative BoNT/B translocation domain variant, a non-conservative BoNT/B translocation domain variant, a BoNT/B chimeric translocation domain, or an active BoNT/B translocation domain fragment; a non-naturally occurring BoNT/C1 translocation domain variant, such as, e.g., a conservative BoNT/C1 translocation domain variant, a non-conservative BoNT/C1 translocation domain variant, a BoNT/C1 chimeric translocation domain, or an active BoNT/C1 translocation domain fragment; a non-naturally occurring BoNT/D translocation domain variant, such as, e.g., a conservative BoNT/D translocation domain variant, a non-conservative BoNT/D translocation domain variant, a BoNT/D chimeric translocation domain, or an active BoNT/D translocation domain fragment; a non-naturally occurring BoNT/E translocation domain variant, such as, e.g., a conservative BoNT/E translocation domain variant, a non-conservative BoNT/E translocation domain variant, a BoNT/E chimeric translocation domain, or an active BoNT/E translocation domain fragment; a non-naturally occurring BoNT/F translocation domain variant, such as, e.g., a conservative BoNT/F translocation domain variant, a non-conservative BoNT/F translocation domain variant, a BoNT/F chimeric translocation domain, or an active BoNT/F translocation domain fragment; a non-naturally occurring BoNT/G translocation domain variant, such as, e.g., a conservative BoNT/G translocation domain variant, a non-conservative BoNT/G translocation domain variant, a BoNT/G chimeric translocation domain, or an active BoNT/G translocation domain fragment; a non-naturally occurring TeNT translocation domain variant, such as, e.g., a conservative TeNT translocation domain variant, a non-conservative TeNT translocation domain variant, a TeNT chimeric translocation domain, or an active TeNT translocation domain fragment; a non-naturally occurring BaNT translocation domain variant, such as, e.g., a conservative BaNT translocation domain variant, a non-conservative BaNT translocation domain variant, a BaNT chimeric translocation domain, or an active BaNT translocation domain fragment; or a non-naturally occurring BuNT translocation domain variant, such as, e.g., a conservative BuNT translocation domain variant, a non-conservative BuNT translocation domain variant, a BuNT chimeric translocation domain, or an active BuNT translocation domain fragment.

In aspects of this embodiment, a non-naturally occurring Clostridial toxin translocation domain variant is a polypeptide having an amino acid identity to the reference Clostridial toxin translocation domain on which the non-naturally occurring Clostridial toxin translocation domain variant is based of, e.g., at least 70%, at least 75%, at least 80%, at least 85%, at least 90% or at least 95%. In yet other aspects of this embodiment, a non-naturally occurring Clostridial toxin translocation domain variant is a polypeptide having an amino acid identity to the reference Clostridial toxin translocation domain on which the non-naturally occurring Clostridial toxin translocation domain variant is based of, e.g., at most 70%, at most 75%, at most 80%, at most 85%, at most 90% or at most 95%.

In other aspects of this embodiment, a non-naturally occurring Clostridial toxin translocation domain variant is a polypeptide having, e.g., at most 1, 2, 3, 4, 5, 6, 7, 8, 9, 10, 20, 30, 40, 50, or 100 non-contiguous amino acid substitutions relative to the reference Clostridial toxin translocation domain on which the non-naturally occurring Clostridial toxin translocation domain variant is based; at least 1, 2, 3, 4, 5, 6, 7, 8, 9, 10, 20, 30, 40, 50, or 100 non-contiguous amino acid substitutions relative to the reference Clostridial toxin translocation domain on which the non-naturally occurring Clostridial toxin translocation domain variant is based; at most 1, 2, 3, 4, 5, 6, 7, 8, 9, 10, 20, 30, 40, 50, or 100 non-contiguous amino acid deletions relative to the reference Clostridial toxin translocation domain on which the non-naturally occurring Clostridial toxin translocation domain variant is based; at least 1, 2, 3, 4, 5, 6, 7, 8, 9, 10, 20, 30, 40, 50, or 100 non-contiguous amino acid deletions relative to the reference Clostridial toxin translocation domain on which the non-naturally occurring Clostridial toxin translocation domain variant is based; at most 1, 2, 3, 4, 5, 6, 7, 8, 9, 10, 20, 30, 40, 50, or 100 non-contiguous amino acid additions relative to the reference Clostridial toxin translocation domain on which the non-naturally occurring Clostridial toxin translocation domain variant is based; or at least 1, 2, 3, 4, 5, 6, 7, 8, 9, 10, 20, 30, 40, 50, or 100 non-contiguous amino acid additions relative to the reference Clostridial toxin translocation domain on which the non-naturally occurring Clostridial toxin translocation domain variant is based.

In yet other aspects of this embodiment, a non-naturally occurring Clostridial toxin translocation domain variant is a polypeptide having, e.g., at most 1, 2, 3, 4, 5, 6, 7, 8, 9, 10, 20, 30, 40, 50, or 100 contiguous amino acid substitutions relative to the reference Clostridial toxin translocation domain on which the non-naturally occurring Clostridial toxin translocation domain variant is based; at least 1, 2, 3, 4, 5, 6, 7, 8, 9, 10, 20, 30, 40, 50, or 100 contiguous amino acid substitutions relative to the reference Clostridial toxin translocation domain on which the non-naturally occurring Clostridial toxin translocation domain variant is based; at most 1, 2, 3, 4, 5, 6, 7, 8, 9, 10, 20, 30, 40, 50, or 100 contiguous amino acid deletions relative to the reference Clostridial toxin translocation domain on which the non-naturally occurring Clostridial toxin translocation domain variant is based; at least 1, 2, 3, 4, 5, 6, 7, 8, 9, 10, 20, 30, 40, 50, or 100 contiguous amino acid deletions relative to the reference Clostridial toxin translocation domain on which the non-naturally occurring Clostridial toxin translocation domain variant is based; at most 1, 2, 3, 4, 5, 6, 7, 8, 9, 10, 20, 30, 40, 50, or 100 contiguous amino acid additions relative to the reference Clostridial toxin translocation domain on which the non-naturally occurring Clostridial toxin translocation domain variant is based; or at least 1, 2, 3, 4, 5, 6, 7, 8, 9, 10, 20, 30, 40, 50, or 100 contiguous amino acid additions relative to the reference Clostridial toxin translocation domain on which the non-naturally occurring Clostridial toxin translocation domain variant is based.

In another embodiment, a hydrophic amino acid at one particular position in the polypeptide chain of the Clostridial toxin translocation domain variant can be substituted with another hydrophic amino acid. Examples of hydrophic amino acids include, e.g., C, F, I, L, M, V and W. In another aspect of this embodiment, an aliphatic amino acid at one particular position in the polypeptide chain of the Clostridial toxin translocation domain variant can be substituted with another aliphatic amino acid. Examples of aliphatic amino acids include, e.g., A, I, L, P, and V. In yet another aspect of this embodiment, an aromatic amino acid at one particular position in the polypeptide chain of the Clostridial toxin translocation domain variant can be substituted with another aromatic amino acid. Examples of aromatic amino acids include, e.g., F, H, W and Y. In still another aspect of this embodiment, a stacking amino acid at one particular position in the polypeptide chain of the Clostridial toxin translocation domain variant can be substituted with another stacking amino acid. Examples of stacking amino acids include, e.g., F, H, W and Y. In a further aspect of this embodiment, a polar amino acid at one particular position in the polypeptide chain of the Clostridial toxin translocation domain variant can be substituted with another polar amino acid. Examples of polar amino acids include, e.g., D, E, K, N, Q, and R. In a further aspect of this embodiment, a less polar or indifferent amino acid at one particular position in the polypeptide chain of the Clostridial toxin translocation domain variant can be substituted with another less polar or indifferent amino acid. Examples of less polar or indifferent amino acids include, e.g., A, H, G, P, S, T, and Y. In a yet further aspect of this embodiment, a positive charged amino acid at one particular position in the polypeptide chain of the Clostridial toxin translocation domain variant can be substituted with another positive charged amino acid. Examples of positive charged amino acids include, e.g., K, R, and H. In a still further aspect of this embodiment, a negative charged amino acid at one particular position in the polypeptide chain of the Clostridial toxin translocation domain variant can be substituted with another negative charged amino acid. Examples of negative charged amino acids include, e.g., D and E. In another aspect of this embodiment, a small amino acid at one particular position in the polypeptide chain of the Clostridial toxin translocation domain variant can be substituted with another small amino acid. Examples of small amino acids include, e.g., A, D, G, N, P, S, and T. In yet another aspect of this embodiment, a C-beta branching amino acid at one particular position in the polypeptide chain of the Clostridial toxin translocation domain variant can be substituted with another C-beta branching amino acid. Examples of C-beta branching amino acids include, e.g., I, T and V.

Any of a variety of sequence alignment methods can be used to determine percent identity of naturally-occurring Clostridial toxin enzymatic domain variants, non-naturally-occurring Clostridial toxin enzymatic domain variants, naturally-occurring Clostridial toxin translocation domain variants, non-naturally-occurring Clostridial toxin translocation domain variants, and binding domains, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics, 1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based On Segment-To-Segment Comparison, 93(22) Proc. Natl. Acad. Sci. U.S.A. 12098-12103 (1996); T-Coffee, see, e.g., Cédric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

It is understood that a modified Clostridial toxin disclosed in the present specification can optionally further comprise a flexible region comprising a flexible spacer. A flexible region comprising flexible spacers can be used to adjust the length of a polypeptide region in order to optimize a characteristic, attribute or property of a polypeptide. As a non-limiting example, a polypeptide region comprising one or more flexible spacers in tandem can be used to better expose a protease cleavage site thereby facilitating cleavage of that site by a protease. As another non-limiting example, a polypeptide region comprising one or more flexible spacers in tandem can be used to better present an integrated protease cleavage site-binding domain, thereby facilitating the binding of that binding domain to its receptor.

A flexible space comprising a peptide is at least one amino acid in length and comprises non-charged amino acids with small side-chain R groups, such as, e.g., glycine, alanine, valine, leucine, serine, or histine. Thus, in an embodiment a flexible spacer can have a length of, e.g., at least 1 amino acids, at least 2 amino acids, at least 3 amino acids, at least 4 amino acids, at least 5 amino acids, at least 6 amino acids, at least 7 amino acids, at least 8 amino acids, at least 9 amino acids, or at least 10 amino acids. In another embodiment, a flexible spacer can have a length of, e.g., at most 1 amino acids, at most 2 amino acids, at most 3 amino acids, at most 4 amino acids, at most 5 amino acids, at most 6 amino acids, at most 7 amino acids, at most 8 amino acids, at most 9 amino acids, or at most 10 amino acids. In still another embodiment, a flexible spacer can be, e.g., between 1-3 amino acids, between 2-4 amino acids, between 3-5 amino acids, between 4-6 amino acids, or between 5-7 amino acids. Non-limiting examples of a flexible spacer include, e.g., a G-spacers such as GGG, GGGG (SEQ ID NO: 144), and GGGGS (SEQ ID NO: 145) or an A-spacers such as AAA, AAAA (SEQ ID NO: 146) and AAAAV (SEQ ID NO: 147). Such a flexible region is operably-linked in-frame to the modified Clostridial toxin as a fusion protein.

Thus, in an embodiment, a modified Clostridial toxin disclosed in the present specification can further comprise a flexible region comprising a flexible spacer. In another embodiment, a modified Clostridial toxin disclosed in the present specification can further comprise flexible region comprising a plurality of flexible spacers in tandem. In aspects of this embodiment, a flexible region can comprise in tandem, e.g., at least 1 G-spacer, at least 2 G-spacers, at least 3 G-spacers, at least 4 G-spacers or at least 5 G-spacers. In other aspects of this embodiment, a flexible region can comprise in tandem, e.g., at most 1 G-spacer, at most 2 G-spacers, at most 3 G-spacers, at most 4 G-spacers or at most 5 G-spacers. In still other aspects of this embodiment, a flexible region can comprise in tandem, e.g., at least 1 A-spacer, at least 2 A-spacers, at least 3 A-spacers, at least 4 A-spacers or at least 5 A-spacers. In still other aspects of this embodiment, a flexible region can comprise in tandem, e.g., at most 1 A-spacer, at most 2 A-spacers, at most 3 A-spacers, at most 4 A-spacers or at most 5 A-spacers. In another aspect of this embodiment, a modified Clostridial toxin can comprise a flexible region comprising one or more copies of the same flexible spacers, one or more copies of different flexible-spacer regions, or any combination thereof.

In other aspects of this embodiment, a modified Clostridial toxin comprising a flexible spacer can be, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

It is envisioned that a modified Clostridial toxin disclosed in the present specification can comprise a flexible spacer in any and all locations with the proviso that modified Clostridial toxin is capable of performing the intoxication process. In aspects of this embodiment, a flexible spacer is positioned between, e.g., an enzymatic domain and a translocation domain, an enzymatic domain and an integrated protease cleavage site-binding domain, an enzymatic domain and an exogenous protease cleavage site. In other aspects of this embodiment, a G-spacer is positioned between, e.g., an enzymatic domain and a translocation domain, an enzymatic domain and an integrated protease cleavage site-binding domain, an enzymatic domain and an exogenous protease cleavage site. In other aspects of this embodiment, an A-spacer is positioned between, e.g., an enzymatic domain and a translocation domain, an enzymatic domain and an integrated protease cleavage site-binding domain, an enzymatic domain and an exogenous protease cleavage site.

In other aspects of this embodiment, a flexible spacer is positioned between, e.g., an integrated protease cleavage site-binding domain and a translocation domain, an integrated protease cleavage site-binding domain and an enzymatic domain, an integrated protease cleavage site-binding domain and an exogenous protease cleavage site. In other aspects of this embodiment, a G-spacer is positioned between, e.g., an integrated protease cleavage site-binding domain and a translocation domain, an integrated protease cleavage site-binding domain and an enzymatic domain, an integrated protease cleavage site-binding domain and an exogenous protease cleavage site. In other aspects of this embodiment, an A-spacer is positioned between, e.g., an integrated protease cleavage site-binding domain and a translocation domain, an integrated protease cleavage site-binding domain and an enzymatic domain, an integrated protease cleavage site-binding domain and an exogenous protease cleavage site.

In yet other aspects of this embodiment, a flexible spacer is positioned between, e.g., a translocation domain and an enzymatic domain, a translocation domain and an integrated protease cleavage site-binding domain, a translocation domain and an exogenous protease cleavage site. In other aspects of this embodiment, a G-spacer is positioned between, e.g., a translocation domain and an enzymatic domain, a translocation domain and an integrated protease cleavage site-binding domain, a translocation domain and an exogenous protease cleavage site. In other aspects of this embodiment, an A-spacer is positioned between, e.g., a translocation domain and an enzymatic domain, a translocation domain and an integrated protease cleavage site-binding domain, a translocation domain and an exogenous protease cleavage site.

It is envisioned that a modified Clostridial toxin disclosed in the present specification can comprise an integrated protease cleavage site-binding domain in any and all locations with the proviso that modified Clostridial toxin is capable of performing the intoxication process. Non-limiting examples include, locating an integrated protease cleavage site-binding domain at the amino terminus of a modified Clostridial toxin; and locating an integrated protease cleavage site-binding domain between a Clostridial toxin enzymatic domain and a translocation domain of a modified Clostridial toxin. Other non-limiting examples include, locating an integrated protease cleavage site-binding domain between a Clostridial toxin enzymatic domain and a Clostridial toxin translocation domain of a modified Clostridial toxin. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where an integrated protease cleavage site-binding domain is in the amino-terminal position, an amino acid sequence comprising a start methionine and a protease cleavage site may be operably-linked in situations in which an integrated protease cleavage site-binding domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

Thus, in an embodiment, a modified Clostridial toxin disclosed in the present specification can comprise an amino to carboxyl single polypeptide linear order comprising an integrated protease cleavage site-binding domain, a Clostridial toxin translocation domain, and a Clostridial toxin enzymatic domain. In another embodiment, a modified Clostridial toxin disclosed in the present specification can comprise an amino to carboxyl single polypeptide linear order comprising an integrated protease cleavage site-binding domain, a Clostridial toxin enzymatic domain, and a Clostridial toxin translocation domain. In yet another embodiment, a modified Clostridial toxin disclosed in the present specification can comprise an amino to carboxyl single polypeptide linear order comprising a Clostridial toxin enzymatic domain, an integrated protease cleavage site-binding domain, and a Clostridial toxin translocation domain. In yet another embodiment, a modified Clostridial toxin disclosed in the present specification can comprise an amino to carboxyl single polypeptide linear order comprising a Clostridial toxin translocation domain, an integrated protease cleavage site-binding domain, and a Clostridial toxin enzymatic domain.

Aspects of the present invention provide, in part, polynucleotide molecules. As used herein, the term “polynucleotide molecule” is synonymous with “nucleic acid molecule” and means a polymeric form of nucleotides, such as, e.g., ribonucleotides and deoxyribonucleotides, of any length. It is envisioned that any and all modified Clostridial toxin disclosed in the present specification can be encoded by a polynucleotide molecule. It is also envisioned that any and all polynucleotide molecules that can encode a modified Clostridial toxin disclosed in the present specification can be useful, including, without limitation naturally-occurring and non-naturally-occurring DNA molecules and naturally-occurring and non-naturally-occurring RNA molecules. Non-limiting examples of naturally-occurring and non-naturally-occurring DNA molecules include single-stranded DNA molecules, double-stranded DNA molecules, genomic DNA molecules, cDNA molecules, vector constructs, such as, e.g., plasmid constructs, phagmid constructs, bacteriophage constructs, retroviral constructs and artificial chromosome constructs. Non-limiting examples of naturally-occurring and non-naturally-occurring RNA molecules include single-stranded RNA, double stranded RNA and mRNA.

Well-established molecular biology techniques that may be necessary to make a polynucleotide molecule encoding a modified Clostridial toxin disclosed in the present specification include, but not limited to, procedures involving polymerase chain reaction (PCR) amplification, restriction enzyme reactions, agarose gel electrophoresis, nucleic acid ligation, bacterial transformation, nucleic acid purification, nucleic acid sequencing and recombination-based techniques that are routine procedures well within the scope of one skilled in the art and from the teaching herein. Non-limiting examples of specific protocols necessary to make a polynucleotide molecule encoding a modified Clostridial toxin are described in e.g., MOLECULAR CLONING A LABORATORY MANUAL, supra, (2001); and CURRENT PROTOCOLS IN MOLECULAR BIOLOGY (Frederick M. Ausubel et al., eds. John Wiley & Sons, 2004). Additionally, a variety of commercially available products useful for making a polynucleotide molecule encoding a modified Clostridial toxin are widely available. These protocols are routine procedures well within the scope of one skilled in the art and from the teaching herein.

Thus, in an embodiment, a polynucleotide molecule encodes a modified Clostridial toxin disclosed in the present specification. In an aspect of this embodiment, a polynucleotide molecule encodes a modified Clostridial toxin comprising an integrated protease cleavage site-binding domain, a Clostridial toxin translocation domain and a Clostridial toxin enzymatic domain. In another aspect of this embodiment, a polynucleotide molecule encodes a modified Clostridial toxin comprising an integrated protease cleavage site-binding domain, a Clostridial toxin enzymatic domain, and a Clostridial toxin translocation domain. In yet another aspect of this embodiment, a polynucleotide molecule encodes a modified Clostridial toxin comprising a Clostridial toxin enzymatic domain, an integrated protease cleavage site-binding domain, and a Clostridial toxin translocation domain. In still another aspect of this embodiment, a polynucleotide molecule encodes a modified Clostridial toxin comprising a Clostridial toxin translocation domain, an integrated protease cleavage site-binding domain, and a Clostridial toxin enzymatic domain.

Another aspect of the present invention provides, in part, a method of producing a modified Clostridial toxin disclosed in the present specification, such method comprising the step of expressing a polynucleotide molecule encoding a modified Clostridial toxin in a cell. Another aspect of the present invention provides a method of producing a modified Clostridial toxin disclosed in the present specification, such method comprising the steps of introducing an expression construct comprising a polynucleotide molecule encoding a modified Clostridial toxin disclosed in the present specification into a cell and expressing the expression construct in the cell.

The methods disclosed in the present specification include, in part, a modified Clostridial toxin. It is envisioned that any and all modified Clostridial toxins disclosed in the present specification can be produced using the methods disclosed in the present specification. It is also envisioned that any and all polynucleotide molecules encoding a modified Clostridial toxins disclosed in the present specification can be useful in producing a modified Clostridial toxins disclosed in the present specification using the methods disclosed in the present specification.

The methods disclosed in the present specification include, in part, an expression construct. An expression construct comprises a polynucleotide molecule disclosed in the present specification operably-linked to an expression vector useful for expressing the polynucleotide molecule in a cell or cell-free extract. A wide variety of expression vectors can be employed for expressing a polynucleotide molecule encoding a modified Clostridial toxin, including, without limitation, a viral expression vector; a prokaryotic expression vector; eukaryotic expression vectors, such as, e.g., a yeast expression vector, an insect expression vector and a mammalian expression vector; and a cell-free extract expression vector. It is further understood that expression vectors useful to practice aspects of these methods may include those which express a modified Clostridial toxin under control of a constitutive, tissue-specific, cell-specific or inducible promoter element, enhancer element or both. Non-limiting examples of expression vectors, along with well-established reagents and conditions for making and using an expression construct from such expression vectors are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; EMD Biosciences-Novagen, Madison, Wis.; QIAGEN, Inc., Valencia, Calif.; and Stratagene, La Jolla, Calif. The selection, making and use of an appropriate expression vector are routine procedures well within the scope of one skilled in the art and from the teachings herein.

Thus, aspects of this embodiment include, without limitation, a viral expression vector operably-linked to a polynucleotide molecule encoding a modified Clostridial toxin; a prokaryotic expression vector operably-linked to a polynucleotide molecule encoding a modified Clostridial toxin; a yeast expression vector operably-linked to a polynucleotide molecule encoding a modified Clostridial toxin; an insect expression vector operably-linked to a polynucleotide molecule encoding a modified Clostridial toxin; and a mammalian expression vector operably-linked to a polynucleotide molecule encoding a modified Clostridial toxin. Other aspects of this embodiment include, without limitation, expression constructs suitable for expressing a modified Clostridial toxin disclosed in the present specification using a cell-free extract comprising a cell-free extract expression vector operably linked to a polynucleotide molecule encoding a modified Clostridial toxin.

The methods disclosed in the present specification include, in part, a cell. It is envisioned that any and all cells can be used. Thus, aspects of this embodiment include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection, European Collection of Cell Cultures and the German Collection of Microorganisms and Cell Cultures. Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4^{th} ed. 2000); ANIMAL CELL CULTURE: A PRACTICAL APPROACH (John R. W. Masters ed., Oxford University Press, 3^{rd} ed. 2000); MOLECULAR CLONING A LABORATORY MANUAL, supra, (2001); BASIC CELL CULTURE: A PRACTICAL APPROACH (John M. Davis, Oxford Press, 2^{nd} ed. 2002); and CURRENT PROTOCOLS IN MOLECULAR BIOLOGY, supra, (2004). These protocols are routine procedures within the scope of one skilled in the art and from the teaching herein.

The methods disclosed in the present specification include, in part, introducing into a cell a polynucleotide molecule. A polynucleotide molecule introduced into a cell can be transiently or stably maintained by that cell. Stably-maintained polynucleotide molecules may be extra-chromosomal and replicate autonomously, or they may be integrated into the chromosomal material of the cell and replicate non-autonomously. It is envisioned that any and all methods for introducing a polynucleotide molecule disclosed in the present specification into a cell can be used. Methods useful for introducing a nucleic acid molecule into a cell include, without limitation, chemical-mediated transfection or transformation such as, e.g., calcium choloride-mediated, calcium phosphate-mediated, diethyl-aminoethyl (DEAE) dextran-mediated, lipid-mediated, polyethyleneimine (PEI)-mediated, polylysine-mediated and polybrene-mediated; physical-mediated tranfection or transformation, such as, e.g., biolistic particle delivery, microinjection, protoplast fusion and electroporation; and viral-mediated transfection, such as, e.g., retroviral-mediated transfection, see, e.g., Introducing Cloned Genes into Cultured Mammalian Cells, pp. 16.1-16.62 (Sambrook & Russell, eds., Molecular Cloning A Laboratory Manual, Vol. 3, 3^{rd} ed. 2001). One skilled in the art understands that selection of a specific method to introduce an expression construct into a cell will depend, in part, on whether the cell will transiently contain an expression construct or whether the cell will stably contain an expression construct. These protocols are routine procedures within the scope of one skilled in the art and from the teaching herein.

In an aspect of this embodiment, a chemical-mediated method, termed transfection, is used to introduce a polynucleotide molecule encoding a modified Clostridial toxin into a cell. In chemical-mediated methods of transfection the chemical reagent forms a complex with the nucleic acid that facilitates its uptake into the cells. Such chemical reagents include, without limitation, calcium phosphate-mediated, see, e.g., Martin Jordan & Florian Worm, Transfection of adherent and suspended cells by calcium phosphate, 33(2) Methods 136-143 (2004); diethyl-aminoethyl (DEAE) dextran-mediated, lipid-mediated, cationic polymer-mediated like polyethyleneimine (PEI)-mediated and polylysine-mediated and polybrene-mediated, see, e.g., Chun Zhang et al., Polyethylenimine strategies for plasmid delivery to brain-derived cells, 33(2) Methods 144-150 (2004). Such chemical-mediated delivery systems can be prepared by standard methods and are commercially available, see, e.g., CellPhect Transfection Kit (Amersham Biosciences, Piscataway, N.J.); Mammalian Transfection Kit, Calcium phosphate and DEAE Dextran, (Stratagene, Inc., La Jolla, Calif.); LIPOFECTAMINE™ Transfection Reagent (Invitrogen, Inc., Carlsbad, Calif.); ExGen 500 Transfection kit (Fermentas, Inc., Hanover, Md.), and SuperFect and Effectene Transfection Kits (Qiagen, Inc., Valencia, Calif.).

In another aspect of this embodiment, a physical-mediated method is used to introduce a polynucleotide molecule encoding a modified Clostridial toxin into a cell. Physical techniques include, without limitation, electroporation, biolistic and microinjection. Biolistics and microinjection techniques perforate the cell wall in order to introduce the nucleic acid molecule into the cell, see, e.g., Jeike E. Biewenga et al., Plasmid-mediated gene transfer in neurons using the biolistics technique, 71(1) J. Neurosci. Methods 67-75 (1997); and John O'Brien & Sarah C. R. Lummis, Biolistic and diolistic transfection: using the gene gun to deliver DNA and lipophilic dyes into mammalian cells, 33(2) Methods 121-125 (2004). Electroporation, also termed electropermeabilization, uses brief, high-voltage, electrical pulses to create transient pores in the membrane through which the nucleic acid molecules enter and can be used effectively for stable and transient transfections of all cell types, see, e.g., M. Golzio et al., In vitro and in vivo electric field-mediated permeabilization, gene transfer, and expression, 33(2) Methods 126-135 (2004); and Oliver Gresch et al., New non-viral method for gene transfer into primary cells, 33(2) Methods 151-163 (2004).

In another aspect of this embodiment, a viral-mediated method, termed transduction, is used to introduce a polynucleotide molecule encoding a modified Clostridial toxin into a cell. In viral-mediated methods of transient transduction, the process by which viral particles infect and replicate in a host cell has been manipulated in order to use this mechanism to introduce a nucleic acid molecule into the cell. Viral-mediated methods have been developed from a wide variety of viruses including, without limitation, retroviruses, adenoviruses, adeno-associated viruses, herpes simplex viruses, picornaviruses, alphaviruses and baculoviruses, see, e.g., Armin Blesch, Lentiviral and MLV based retroviral vectors for ex vivo and in vivo gene transfer, 33(2) Methods 164-172 (2004); and Maurizio Federico, From lentiviruses to lentivirus vectors, 229 Methods Mol. Biol. 3-15 (2003); E. M. Poeschla, Non-primate lentiviral vectors, 5(5) Curr. Opin. Mol. Ther. 529-540 (2003); Karim Benihoud et al, Adenovirus vectors for gene delivery, 10(5) Curr. Opin. Biotechnol. 440-447 (1999); H. Bueler, Adeno-associated viral vectors for gene transfer and gene therapy, 380(6) Biol. Chem. 613-622 (1999); Chooi M. Lai et al., Adenovirus and adeno-associated virus vectors, 21(12) DNA Cell Biol. 895-913 (2002); Edward A. Burton et al., Gene delivery using herpes simplex virus vectors, 21(12) DNA Cell Biol. 915-936 (2002); Paola Grandi et al., Targeting HSV amplicon vectors, 33(2) Methods 179-186 (2004); Ilya Frolov et al., Alphavirus-based expression vectors: strategies and applications, 93(21) Proc. Natl. Acad. Sci. U.S.A. 11371-11377 (1996); Markus U. Ehrengruber, Alphaviral gene transfer in neurobiology, 59(1) Brain Res. Bull. 13-22 (2002); Thomas A. Kost & J. Patrick Condreay, Recombinant baculoviruses as mammalian cell gene-delivery vectors, 20(4) Trends Biotechnol. 173-180 (2002); and A. Huser & C. Hofmann, Baculovirus vectors: novel mammalian cell gene-delivery vehicles and their applications, 3(1) Am. J. Pharmacogenomics 53-63 (2003).

Adenoviruses, which are non-enveloped, double-stranded DNA viruses, are often selected for mammalian cell transduction because adenoviruses handle relatively large polynucleotide molecules of about 36 kb, are produced at high titer, and can efficiently infect a wide variety of both dividing and non-dividing cells, see, e.g., Wim T. J. M. C. Hermens et al., Transient gene transfer to neurons and glia: analysis of adenoviral vector performance in the CNS and PNS, 71(1) J. Neurosci. Methods 85-98 (1997); and Hiroyuki Mizuguchi et al., Approaches for generating recombinant adenovirus vectors, 52(3) Adv. Drug Deliv. Rev. 165-176 (2001). Transduction using adenoviral-based system do not support prolonged protein expression because the nucleic acid molecule is carried by an episome in the cell nucleus, rather than being integrated into the host cell chromosome. Adenoviral vector systems and specific protocols for how to use such vectors are disclosed in, e.g., VIRAPOWER™ Adenoviral Expression System (Invitrogen, Inc., Carlsbad, Calif.) and VIRAPOWER™ Adenoviral Expression System Instruction Manual 25-0543 version A, Invitrogen, Inc., (Jul. 15, 2002); and ADEASY™ Adenoviral Vector System (Stratagene, Inc., La Jolla, Calif.) and ADEASY™ Adenoviral Vector System Instruction Manual 064004f, Stratagene, Inc.

Nucleic acid molecule delivery can also use single-stranded RNA retroviruses, such as, e.g., oncoretroviruses and lentiviruses. Retroviral-mediated transduction often produce transduction efficiencies close to 100%, can easily control the proviral copy number by varying the multiplicity of infection (MOI), and can be used to either transiently or stably transduce cells, see, e.g., Tiziana Tonini et al., Transient production of retroviral- and lentiviral-based vectors for the transduction of Mammalian cells, 285 Methods Mol. Biol. 141-148 (2004); Armin Blesch, Lentiviral and MLV based retroviral vectors for ex vivo and in vivo gene transfer, 33(2) Methods 164-172 (2004); Félix Recillas-Targa, Gene transfer and expression in mammalian cell lines and transgenic animals, 267 Methods Mol. Biol. 417-433 (2004); and Roland Wolkowicz et al., Lentiviral vectors for the delivery of DNA into mammalian cells, 246 Methods Mol. Biol. 391-411 (2004). Retroviral particles consist of an RNA genome packaged in a protein capsid, surrounded by a lipid envelope. The retrovirus infects a host cell by injecting its RNA into the cytoplasm along with the reverse transcriptase enzyme. The RNA template is then reverse transcribed into a linear, double stranded cDNA that replicates itself by integrating into the host cell genome. Viral particles are spread both vertically (from parent cell to daughter cells via the provirus) as well as horizontally (from cell to cell via virions). This replication strategy enables long-term persistent expression since the nucleic acid molecules of interest are stably integrated into a chromosome of the host cell, thereby enabling long-term expression of the protein. For instance, animal studies have shown that lentiviral vectors injected into a variety of tissues produced sustained protein expression for more than 1 year, see, e.g., Luigi Naldini et al., In vivo gene delivery and stable transduction of non-dividing cells by a lentiviral vector, 272(5259) Science 263-267 (1996). The Oncoretroviruses-derived vector systems, such as, e.g., Moloney murine leukemia virus (MoMLV), are widely used and infect many different non-dividing cells. Lentiviruses can also infect many different cell types, including dividing and non-dividing cells and possess complex envelope proteins, which allows for highly specific cellular targeting.

Retroviral vectors and specific protocols for how to use such vectors are disclosed in, e.g., Manfred Gossen & Hermann Bujard, Tight control of gene expression in eukaryotic cells by tetracycline-responsive promoters, U.S. Pat. No. 5,464,758 (Nov. 7, 1995) and Hermann Bujard & Manfred Gossen, Methods for regulating gene expression, U.S. Pat. No. 5,814,618 (Sep. 29, 1998) David S. Hogness, Polynucleotides encoding insect steroid hormone receptor polypeptides and cells transformed with same, U.S. Pat. No. 5,514,578 (May 7, 1996) and David S. Hogness, Polynucleotide encoding insect ecdysone receptor, U.S. Pat. No. 6,245,531 (Jun. 12, 2001); Elisabetta Vegeto et al., Progesterone receptor having C. terminal hormone binding domain truncations, U.S. Pat. No. 5,364,791 (Nov. 15, 1994), Elisabetta Vegeto et al., Mutated steroid hormone receptors, methods for their use and molecular switch for gene therapy, U.S. Pat. No. 5,874,534 (Feb. 23, 1999) and Elisabetta Vegeto et al., Mutated steroid hormone receptors, methods for their use and molecular switch for gene therapy, U.S. Pat. No. 5,935,934 (Aug. 10, 1999). Furthermore, such viral delivery systems can be prepared by standard methods and are commercially available, see, e.g., BD™ Tet-Off and Tet-On Gene Expression Systems (BD Biosciences-Clonetech, Palo Alto, Calif.) and BD™ Tet-Off and Tet-On Gene Expression Systems User Manual, PT3001-1, BD Biosciences Clonetech, (Mar. 14, 2003), GeneSwitch™ System (Invitrogen, Inc., Carlsbad, Calif.) and GENESWITCH™ System A Mifepristone-Regulated Expression System for Mammalian Cells version D, 25-0313, Invitrogen, Inc., (Nov. 4, 2002); VIRAPOWER™ Lentiviral Expression System (Invitrogen, Inc., Carlsbad, Calif.) and VIRAPOWER™ Lentiviral Expression System Instruction Manual 25-0501 version E, Invitrogen, Inc., (Dec. 8, 2003); and COMPLETE CONTROL® Retroviral Inducible Mammalian Expression System (Stratagene, La Jolla, Calif.) and COMPLETE CONTROL® Retroviral Inducible Mammalian Expression System Instruction Manual, 064005e.

The methods disclosed in the present specification include, in part, expressing a modified Clostridial toxin from a polynucleotide molecule. It is envisioned that any of a variety of expression systems may be useful for expressing a modified Clostridial toxin from a polynucleotide molecule disclosed in the present specification, including, without limitation, cell-based systems and cell-free expression systems. Cell-based systems include, without limitation, viral expression systems, prokaryotic expression systems, yeast expression systems, baculoviral expression systems, insect expression systems and mammalian expression systems. Cell-free systems include, without limitation, wheat germ extracts, rabbit reticulocyte extracts and E. coli extracts and generally are equivalent to the method disclosed herein. Expression of a polynucleotide molecule using an expression system can include any of a variety of characteristics including, without limitation, inducible expression, non-inducible expression, constitutive expression, viral-mediated expression, stably-integrated expression, and transient expression. Expression systems that include well-characterized vectors, reagents, conditions and cells are well-established and are readily available from commercial vendors that include, without limitation, Ambion, Inc. Austin, Tex.; BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; Roche Applied Science, Indianapolis, Ind.; and Stratagene, La Jolla, Calif. Non-limiting examples on the selection and use of appropriate heterologous expression systems are described in e.g., PROTEIN EXPRESSION. A PRACTICAL APPROACH(S. J. Higgins and B. David Hames eds., Oxford University Press, 1999); Joseph M. Fernandez & James P. Hoeffler, GENE EXPRESSION SYSTEMS. USING NATURE FOR THE ART OF EXPRESSION (Academic Press, 1999); and Meena Rai & Harish Padh, Expression Systems for Production of Heterologous Proteins, 80(9) CURRENT SCIENCE 1121-1128, (2001). These protocols are routine procedures well within the scope of one skilled in the art and from the teaching herein.

A variety of cell-based expression procedures are useful for expressing a modified Clostridial toxin encoded by polynucleotide molecule disclosed in the present specification. Examples included, without limitation, viral expression systems, prokaryotic expression systems, yeast expression systems, baculoviral expression systems, insect expression systems and mammalian expression systems. Viral expression systems include, without limitation, the VIRAPOWER™ Lentiviral (Invitrogen, Inc., Carlsbad, Calif.), the Adenoviral Expression Systems (Invitrogen, Inc., Carlsbad, Calif.), the ADEASY™ XL Adenoviral Vector System (Stratagene, La Jolla, Calif.) and the VIRAPORT® Retroviral Gene Expression System (Stratagene, La Jolla, Calif.). Non-limiting examples of prokaryotic expression systems include the CHAMPION™ pET Expression System (EMD Biosciences-Novagen, Madison, Wis.), the TRIEX™ Bacterial Expression System (EMD Biosciences-Novagen, Madison, Wis.), the QIAEXPRESS® Expression System (QIAGEN, Inc.), and the AFFINITY® Protein Expression and Purification System (Stratagene, La Jolla, Calif.). Yeast expression systems include, without limitation, the EASYSELECT™ Pichia Expression Kit (Invitrogen, Inc., Carlsbad, Calif.), the YES-ECHO™ Expression Vector Kits (Invitrogen, Inc., Carlsbad, Calif.) and the SPECTRA™ S. pombe Expression System (Invitrogen, Inc., Carlsbad, Calif.). Non-limiting examples of baculoviral expression systems include the BaculoDirect™ (Invitrogen, Inc., Carlsbad, Calif.), the BAC-TO-BAC® (Invitrogen, Inc., Carlsbad, Calif.), and the BD BACULOGOLD™ (BD Biosciences-Pharmigen, San Diego, Calif.). Insect expression systems include, without limitation, the Drosophila Expression System (DES®) (Invitrogen, Inc., Carlsbad, Calif.), INSECTSELECT™ System (Invitrogen, Inc., Carlsbad, Calif.) and INSECTDIRECT™ System (EMD Biosciences-Novagen, Madison, Wis.). Non-limiting examples of mammalian expression systems include the T-REXT™ (Tetracycline-Regulated Expression) System (Invitrogen, Inc., Carlsbad, Calif.), the FLP-IN™ T-REX™ System (Invitrogen, Inc., Carlsbad, Calif.), the pcDNA™ system (Invitrogen, Inc., Carlsbad, Calif.), the pSecTag2 system (Invitrogen, Inc., Carlsbad, Calif.), the EXCHANGER® System, INTERPLAY™ Mammalian TAP System (Stratagene, La Jolla, Calif.), COMPLETE CONTROL® Inducible Mammalian Expression System (Stratagene, La Jolla, Calif.) and LACSWITCH® II Inducible Mammalian Expression System (Stratagene, La Jolla, Calif.).

Another procedure of expressing a modified Clostridial toxin encoded by polynucleotide molecule disclosed in the present specification employs a cell-free expression system such as, without limitation, prokaryotic extracts and eukaryotic extracts. Non-limiting examples of prokaryotic cell extracts include the RTS 100 E. coli HY Kit (Roche Applied Science, Indianapolis, Ind.), the ActivePro In Vitro Translation Kit (Ambion, Inc., Austin, Tex.), the EcoPro™ System (EMD Biosciences-Novagen, Madison, Wis.) and the EXPRESSWAY™ Plus Expression System (Invitrogen, Inc., Carlsbad, Calif.). Eukaryotic cell extract include, without limitation, the RTS 100 Wheat Germ CECF Kit (Roche Applied Science, Indianapolis, Ind.), the TNT® Coupled Wheat Germ Extract Systems (Promega Corp., Madison, Wis.), the Wheat Germ IVT™ Kit (Ambion, Inc., Austin, Tex.), the Retic Lysate IVT™ Kit (Ambion, Inc., Austin, Tex.), the PROTEINscript® II System (Ambion, Inc., Austin, Tex.) and the TNT® Coupled Reticulocyte Lysate Systems (Promega Corp., Madison, Wis.).

The modified Clostridial toxins disclosed in the present specification are produced by the cell in a single-chain form. In order to achieve full activity, this single-chain form has to be converted into its di-chain form. This conversion process is achieved by proteolytically cleaving the protease cleavage site located within integrated protease cleavage site-binding domain. This conversion process can be performed using a standard in vitro proteolytic cleavage assay or in a cell-based proteolytic cleavage system as described in a companion patent application Ghanshani, et al., Methods of Intracellular Conversion of Single-Chain Proteins into their Di-chain Form, Attorney Docket No. 18469 PROV (BOT), which is hereby incorporated by reference in its entirety.

Aspects of the present invention provide, in part, a composition comprising a modified Clostridial toxin disclosed in the present specification. A composition useful in the invention generally is administered as a pharmaceutically acceptable composition comprising a modified Clostridial toxin disclosed in the present specification. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the modified Clostridial toxins disclosed in the present specification. A pharmaceutical composition comprising a modified Clostridial toxin is useful for medical and veterinary applications. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

It is also envisioned that a pharmaceutical composition comprising a modified Clostridial toxin can optionally include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

It is further envisioned that a pharmaceutical composition disclosed in the present specification can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., PURITE® and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

Thus, in an embodiment, a composition comprises a modified Clostridial toxin disclosed in the present specification. In an aspect of this embodiment, a pharmaceutical composition comprises a modified Clostridial toxin disclosed in the present specification and a pharmacological carrier. In another aspect of this embodiment, a pharmaceutical composition comprises a modified Clostridial toxin disclosed in the present specification and a pharmacological component. In yet another aspect of this embodiment, a pharmaceutical composition comprises a modified Clostridial toxin disclosed in the present specification, a pharmacological carrier and a pharmacological component. In other aspects of this embodiment, a pharmaceutical composition comprises a modified Clostridial toxin disclosed in the present specification and at least one pharmacological carrier, at least one pharmaceutical component, or at least one pharmacological carrier and at least one pharmaceutical component.

Aspects of the present invention can also be described as follows:
1. A single-chain modified Clostridial toxin comprising: a) a Clostridial toxin enzymatic domain capable of executing an enzymatic target modification step of a Clostridial toxin intoxication process; b) a Clostridial toxin translocation domain capable of executing a translocation step of a Clostridial toxin intoxication process; and c) an integrated protease cleavage site-binding domain comprising a P portion of a protease cleavage site including the P₁ site of the scissile bond and a binding domain, wherein the P₁ site of the P portion of a protease cleavage site abuts the amino-end of binding domain thereby creating an integrated protease cleavage site; wherein cleavage of the integrated protease cleavage site-binding domain converts the single-chain modified Clostridial toxin into a di-chain form and produces a binding domain with an amino-terminus capable of binding to its cognate receptor.
2. The modified Clostridial toxin of 1, wherein the modified Clostridial toxin comprises a linear amino-to-carboxyl single polypeptide order of 1) the Clostridial toxin enzymatic domain, the Clostridial toxin translocation domain, and the integrated protease cleavage site-binding domain, 2) the Clostridial toxin enzymatic domain, the integrated protease cleavage site-binding domain, and the Clostridial toxin translocation domain, 3) the integrated protease cleavage site-binding domain, the Clostridial toxin translocation domain, and the Clostridial toxin enzymatic domain, 4) the integrated protease cleavage site-binding domain, the Clostridial toxin enzymatic domain, and the Clostridial toxin translocation domain, or 5) the Clostridial toxin translocation domain, integrated protease cleavage site-binding domain, and the Clostridial toxin enzymatic domain.
3. The modified Clostridial toxin of 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
4. The modified Clostridial toxin of 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
5. The modified Clostridial toxin of 1, wherein the integrated protease cleavage site-binding domain is any one of SEQ ID NO: 4 to SEQ ID NO: 118.
6. The modified Clostridial toxin of Claim 1, wherein the P portion of a protease cleavage site including the P₁ site of the scissile bond is SEQ ID NO: 121, SEQ ID NO: 127, or SEQ ID NO: 130.
7. The modified Clostridial toxin of 1, wherein the binding domain is an opioid peptide.
8. The modified Clostridial toxin of 7, wherein the opioid peptide is an enkephalin, a BAM22 peptide, an endomorphin, an endorphin, a dynorphin, a nociceptin or a rimorphin.
9. The modified Clostridial toxin of 7, wherein the opioid peptide is SEQ ID NO: 154 to SEQ ID NO: 186.
10. The modified Clostridial toxin of 1, wherein the binding domain is a PAR ligand.
11. The modified Clostridial toxin of 9, wherein the PAR ligand is a PAR1, a PAR2, a PAR3, or a PAR4.
12. A pharmaceutical composition comprising a di-chain form of a single-chain modified Clostridial toxin of Claim 1 and a pharmaceutically acceptable carrier, a pharmaceutically acceptable component, or both a pharmaceutically acceptable carrier and a pharmaceutically acceptable component.
13. A polynucleotide molecule encoding a modified Clostridial toxin according to Claim 1.
14. The polynucleotide molecule according to 12, wherein the polynucleotide molecule further comprises an expression vector.
15. A method of producing a modified Clostridial toxin comprising the steps of: a) introducing into a cell a polynucleotide molecule of Claim 13; and b) expressing the polynucleotide molecule.

### EXAMPLES

### Example 1

### Construction of Modified Clostridial Toxin with Integrated Protease Cleavage Site-Binding Domain

The following example illustrates methods useful for constructing any of the modified Clostridial toxins with an integrated protease cleavage site-binding domain disclosed in the present specification.

To construct a modified Clostridial toxin with an amino-terminal free targeting moiety after activation, a re-targeted toxin comprising a nociceptin targeting moiety was modified to replace the existing enterokinase cleavage site and nociceptin targeting moiety with an integrated protease cleavage site-binding domain (IPCS-BD) as disclosed in the present specification. Examples of re-targeted toxins comprising an enterokinase cleavage site and nociceptin targeting moiety are disclosed in, e.g., Steward, U.S. patent application Ser. No. 12/192,900, supra, (2008); Foster, U.S. patent application Ser. No. 11/792,210, supra, (2007); Foster, U.S. patent application Ser. No. 11/791,979, supra, (2007); Dolly, U.S. Pat. No. 7,419,676, supra, (2008), each of which is hereby incorporated by reference in its entirety. For example, a 7.89-kb expression construct comprising polynucleotide molecule of SEQ ID NO: 148 was digested with EcoRI and XbaI, excising the 260 by polynucleotide molecule encoding the enterokinase cleavage site and the nociceptin targeting moiety and the resulting 7.63 kb EcoRI-XbaI fragment was purified using a gel-purification procedure. A 323 by EcoRI-XbaI fragment (SEQ ID NO: 149) encoding the integrated protease cleavage site-Nociceptin of SEQ ID NO: 152 was subcloned into the purified 7.63 kb EcoRI-XbaI fragment using a T4 DNA ligase procedure. The ligation mixture was transformed into electro-competent E. coli BL21(DE3) cells (Edge Biosystems, Gaithersburg, Md.) using an electroporation method, and the cells were plated on 1.5% Luria-Bertani agar plates (pH 7.0) containing 50 μg/mL of kanamycin, and were placed in a 37° C. incubator for overnight growth. Bacteria containing expression constructs were identified as kanamycin resistant colonies. Candidate constructs were isolated using an alkaline lysis plasmid mini-preparation procedure and analyzed by restriction endonuclease digest mapping to determine the presence and orientation of the insert and by DNA sequencing. This cloning strategy yielded a pET29 expression construct comprising the polynucleotide molecule of SEQ ID NO: 150 encoding the BoNT/A-IPCS-Nociceptin of SEQ ID NO: 151.

Alternatively, a polynucleotide molecule based on BoNT/A-IPCS-Nociceptin (SEQ ID NO: 151) comprising the IPCS-Nociceptin of SEQ ID NO: 152 can be synthesized using standard procedures (BlueHeron® Biotechnology, Bothell, Wash.). Oligonucleotides of 20 to 50 bases in length are synthesized using standard phosphoramidite synthesis. These oligonucleotides will be hybridized into double stranded duplexes that are ligated together to assemble the full-length polynucleotide molecule. This polynucleotide molecule will be cloned using standard molecular biology methods into a pUCBHB1 vector at the SmaI site to generate pUCBHB1/BoNT/A-AP4A-Nociceptin. The synthesized polynucleotide molecule is verified by sequencing using Big Dye Terminator™ Chemistry 3.1 (Applied Biosystems, Foster City, Calif.) and an ABI 3100 sequencer (Applied Biosystems, Foster City, Calif.). If desired, an expression optimized polynucleotide molecule based on BoNT/A-IPCS-Nociceptin (SEQ ID NO: 151) can be synthesized in order to improve expression in an Escherichia coli strain. The polynucleotide molecule encoding the BoNT/A-IPCS-Nociceptin can be modified to 1) contain synonymous codons typically present in native polynucleotide molecules of an Escherichia coli strain; 2) contain a G+C content that more closely matches the average G+C content of native polynucleotide molecules found in an Escherichia coli strain; 3) reduce polymononucleotide regions found within the polynucleotide molecule; and/or 4) eliminate internal regulatory or structural sites found within the polynucleotide molecule, see, e.g., Lance E. Steward et al., Optimizing Expression of Active Botulinum Toxin Type A, U.S. Patent Publication 2008/0057575 (Mar. 6, 2008); and Lance E. Steward et al., Optimizing Expression of Active Botulinum Toxin Type E, U.S. Patent Publication 2008/0138893 (Jun. 12, 2008). Once sequence optimization is complete, oligonucleotides of 20 to 50 bases in length are synthesized using standard phosphoramidite synthesis. These oligonucleotides are hybridized into double stranded duplexes that are ligated together to assemble the full-length polynucleotide molecule. This polynucleotide molecule is cloned using standard molecular biology methods into a pUCBHB1 vector at the SmaI site to generate pUCBHB1/BoNT/A-IPCS-Nociceptin. The synthesized polynucleotide molecule is verified by DNA sequencing. If so desired, expression optimization to a different organism, such as, e.g., a yeast strain, an insect cell-line or a mammalian cell line, can be done, see, e.g., Steward, U.S. Patent Publication 2008/0057575, supra, (2008); and Steward, U.S. Patent Publication 2008/0138893, supra, (2008).

Similar cloning strategies will be used to make pUCBHB1 cloning constructs comprising a polynucleotide molecule encoding BoNT/A-IPCS-BDs comprising other IPCS-BDs, such as, e.g., BoNT/A-IPCS-Enkephalins based on SEQ ID NO: 4-7; BoNT/A-IPCS-BAM-22s based on SEQ ID NO: 8-27; BoNT/A-IPCS-Endomorphins based on SEQ ID NO: 28-29; BoNT/A-IPCS-Endorphins based on SEQ ID NO: 30-35; BoNT/A-IPCS-Dynorphins based on SEQ ID NO: 36-68; BoNT/A-IPCS-Rimorphins based on SEQ ID NO: 69-74; BoNT/A-IPCS-Nociceptins based on SEQ ID NO: 75-84; BoNT/A-IPCS-Neuropeptides based on SEQ ID NO: 85-87; or BoNT/A-IPCS-PARs based on SEQ ID NO: 88-118. Likewise, similar cloning strategies can be used to make pUCBHB1 cloning constructs comprising a polynucleotide molecule encoding for other Clostridial toxin-IPCS-BDs, such as, e.g., a BoNT/B-IPCS-BD, a BoNT/C1-IPCS-BD, a BoNT/D-IPCS-BD, a BoNT/E-IPCS-BD, a BoNT/F-IPCS-BD, a BoNT/G-IPCS-BD, a TeNT-IPCS-BD, a BaNT/B-IPCS-BD, or a BuNT/B-IPCS-BD.

To construct pET29/BoNT/A-IPCS-Nociceptin, a pUCBHB1/BoNT/A-IPCS-Nociceptin construct was digested with restriction endonucleases that 1) excised the polynucleotide molecule encoding the open reading frame of BoNT/A-IPCS-Nociceptin; and 2) enabled this polynucleotide molecule to be operably-linked to a pET29 vector (EMD Biosciences-Novagen, Madison, Wis.). This insert was subcloned using a T4 DNA ligase procedure into a pET29 vector that was digested with appropriate restriction endonucleases to yield pET29/BoNT/A-IPCS-Nociceptin. The ligation mixture was transformed into electro-competent E. coli BL21(DE3) cells (Edge Biosystems, Gaitherburg, Md.) using an electroporation method, and the cells were plated on 1.5% Luria-Bertani agar plates (pH 7.0) containing 50 μg/mL of kanamycin, and were placed in a 37° C. incubator for overnight growth. Bacteria containing expression constructs were identified as kanamycin resistant colonies. Candidate constructs were isolated using an alkaline lysis plasmid mini-preparation procedure and were analyzed by restriction endonuclease digest mapping to determine the presence and orientation of the insert. This cloning strategy yielded a pET29 expression construct comprising the polynucleotide molecule encoding the BoNT/A-IPCS-Nociceptin.

Similar cloning strategies will be used to make pET29 expression constructs comprising a polynucleotide molecule encoding for other BoNT/A-IPCS-BDs, such as, e.g., BoNT/A-IPCS-Enkephalins based on SEQ ID NO: 4-7; BoNT/A-IPCS-BAM-22s based on SEQ ID NO: 8-27; BoNT/A-IPCS-Endomorphins based on SEQ ID NO: 28-29; BoNT/A-IPCS-Endorphins based on SEQ ID NO: 30-35; BoNT/A-IPCS-Dynorphins based on SEQ ID NO: 36-68; BoNT/A-IPCS-Rimorphins based on SEQ ID NO: 69-74; BoNT/A-IPCS-Nociceptins based on SEQ ID NO: 75-84; BoNT/A-IPCS-Neuropeptides based on SEQ ID NO: 85-87; or BoNT/A-IPCS-PARs based on SEQ ID NO: 88-118. Likewise, similar cloning strategies can be used to make pET29 expression constructs comprising a polynucleotide molecule encoding for other Clostridial toxin-IPCS-BDs, such as, e.g., a BoNT/B-IPCS-BD, a BoNT/C1-IPCS-BD, a BoNT/D-IPCS-BD, a BoNT/E-IPCS-BD, a BoNT/F-IPCS-BD, a BoNT/G-IPCS-BD, a TeNT-IPCS-BD, a BaNT/B-IPCS-BD, or a BuNT/B-IPCS-BD.

### Example 2

### Expression of Modified Clostridial Toxin with Integrated Protease Cleavage Site-Binding Domain

The following example illustrates a procedure useful for expressing any of the modified Clostridial toxins disclosed in the present specification in a bacterial cell.

To express a modified Clostridial toxin disclosed in the present specification, an expression construct, such as, e.g., as described in Example 1, was transformed into electro-competent ACELLA® E. coli BL21 (DE3) cells (Edge Biosystems, Gaithersburg, Md.) using an electroporation method. The cells were then be plated onto 1.5% Luria-Bertani agar plates (pH 7.0) containing 50 μg/mL of kanamycin and were placed in a 37° C. incubator for overnight growth. Kanamycin-resistant colonies of transformed E. coli containing the expression construct were used to inoculate a baffled flask containing 3.0 mL of PA-0.5G media containing 50 μg/mL of kanamycin which was then placed in a 37° C. incubator, shaking at 250 rpm, for overnight growth. The resulting overnight starter culture was used to inoculate 250 mL of ZYP-5052 autoinducing media containing 50 μg/mL of kanamycin. These cultures were grown in a 37° C. incubator shaking at 250 rpm for approximately 3.5 hours and were then transferred to a 22° C. incubator shaking at 250 rpm for an additional incubation of 16-18 hours. Cells were harvested by centrifugation (4,000 rpm at 4° C. for 20-30 minutes) and were used immediately, or stored dry at −80° C. until needed.

### Example 3

### Purification of Modified Clostridial Toxin with Integrated Protease Cleavage Site-Binding Domain

The following example illustrates methods useful for purifying and quantifying any of the modified Clostridial toxins disclosed in the present specification.

To lyse cell pellets containing a modified Clostridial toxin disclosed in the present specification, a cell pellet, such as, e.g., as described in Example 2, was resuspended in a lysis buffer containing BUGBUSTER® Protein Extraction Reagent (EMD Biosciences-Novagen, Madison, Wis.); 1× Protease Inhibitor Cocktail Set III (EMD Biosciences-Calbiochem, San Diego Calif.); 25 unit/mL Benzonase nuclease (EMD Biosciences-Novagen, Madison, Wis.); and 1,000 units/mL rLysozyme (EMD Biosciences-Novagen, Madison, Wis.). The cell suspension was incubated at room temperature on a platform rocker for 20 minutes, incubated on ice for 15 minutes to precipitate detergent, than centrifuged at 30,500 rcf for 30 minutes at 4° C. to remove insoluble debris. The clarified supernatant was transferred to a new tube and was used immediately for IMAC purification, or stored dry at 4° C. until needed.

To purify a modified Clostridial toxin disclosed in the present specification using immobilized metal affinity chromatography (IMAC), the clarified supernatant was mixed with 2.5-5.0 mL of TALON™ SuperFlow Co²⁺ affinity resin (BD Biosciences-Clontech, Palo Alto, Calif.) equilibrated with IMAC Wash Buffer (25 mM N-(2-hydroxyethyl) piperazine-N′-(2-ethanesulfonic acid) (HEPES), pH 8.0; 500 mM sodium chloride; 10 mM imidazole; 10% (v/v) glycerol). The clarified supernatant-resin mixture was incubated on a platform rocker for 60 minutes at 4° C. The clarified supernatant-resin mixture was then transferred to a disposable polypropylene column support (Thomas Instruments Co., Philadelphia, Pa.) and attached to a vacuum manifold. The column was washed twice with five column volumes of IMAC Wash Buffer. The modified Clostridial toxin was eluted with 2 column volumes of IMAC Elution Buffer (25 mM N-(2-hydroxyethyl) piperazine-N′-(2-ethanesulfonic acid) (HEPES), pH 8.0; 500 mM sodium chloride; 500 mM imidazole; 10% (v/v) glycerol) and collected in approximately 1 mL fractions. The amount of modified Clostridial toxin contained in each elution fraction was determined by a Bradford dye assay. In this procedure, a 10 μL aliquots of each 1.0 mL fraction was combined with 200 μL of Bio-Rad Protein Reagent (Bio-Rad Laboratories, Hercules, Calif.), diluted 1 to 4 with deionized, distilled water, and the intensity of the colorimetric signal was measured using a spectrophotometer. The fractions with the strongest signal were considered the elution peak and were combined together and dialyzed to adjust the solution for subsequent procedures. Buffer exchange of IMAC-purified modified Clostridial toxin was accomplished by dialysis at 4° C. in a FASTDIALYZER® (Harvard Apparatus) fitted with 25 kD MWCO membranes (Harvard Apparatus). The protein samples were exchanged into the appropriate Desalting Buffer (50 mM Tris-HCl (pH 8.0) to be used in the subsequent ion exchange chromatography purification step. The FASTDIALYZER® was placed in 1 L Desalting Buffer with constant stirring and incubated overnight at 4° C.

For purification of a modified Clostridial toxin disclosed in the present specification using FPLC ion exchange chromatography, the modified Clostridial toxin sample was dialyzed into 50 mM Tris-HCl (pH 8.0) was applied to a 1 mL UNO-Q1™ anion exchange column (Bio-Rad Laboratories, Hercules, Calif.) equilibrated with 50 mM Tris-HCl (pH 8.0) at a flow rate of 0.5 mL/min using a BioLogic DuoFlow chromatography system (Bio-Rad Laboratories, Hercules, Calif.). Bound protein was eluted by NaCl step gradient with elution buffer comprising 50 mM Tris-HCl (pH 8.0); 1 M NaCl at a flow rate of 1.0 ml/min at 4° C. as follows: 3 mL of 7% elution buffer at a flow rate of 1.0 mL/min, 6 mL of 12% elution buffer at a flow rate of 1.0 mL/min, and 10 mL of 12% to 100% elution buffer at a flow rate of 1.0 mL/min. Elution of material from the column was detected with a QuadTec UV-Vis detector at 214 nm, 260 nm and 280 nm, and all peaks absorbing at or above 0.01 AU at 280 nm were collected in 1.0 mL fractions. A standard Typhoon Gel Quatification (GE Healthcare, Piscataway, N.J.) was used to determine protein concentration. Peak fractions were pooled, 5% (v/v) PEG-400 was added, and aliquots were frozen in liquid nitrogen and stored at −80° C.

Expression of a modified Clostridial toxin disclosed in the present specification was analyzed by polyacrylamide gel electrophoresis. Samples of modified Clostridial toxin, purified using the procedure described above, are added to 2×LDS Sample Buffer (Invitrogen, Inc, Carlsbad, Calif.) with and without DTT and separated by MOPS polyacrylamide gel electrophoresis using NuPAGE® Novex 4-12% Bis-Tris precast polyacrylamide gels (Invitrogen, Inc, Carlsbad, Calif.) under denaturing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.). To quantify modified Clostridial toxin yield, varying amounts of purified modified Clostridial toxin samples were added to 2×LDS Sample Buffer (Invitrogen, Inc, Carlsbad, Calif.) without DTT and were separated on by MOPS polyacrylamide gel electrophoresis using NuPAGE® Novex 4-12% Bis-Tris precast polyacrylamide gels (Invitrogen, Inc, Carlsbad, Calif.) under non-reducing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.). Following imaging, a reference curve is plotted for the BSA standards and the toxin quantities interpolated from this curve. The size of modified Clostridial toxin was determined by comparison to MagicMark™ protein molecular weight standards (Invitrogen, Inc, Carlsbad, Calif.).

Expression of a modified Clostridial toxin disclosed in the present specification was also analyzed by Western blot analysis. Protein samples purified using the procedure described above were added to 2×LDS Sample Buffer (Invitrogen, Inc, Carlsbad, Calif.) with and without DTT and separated by MOPS polyacrylamide gel electrophoresis using NuPAGE® Novex 4-12% Bis-Tris precast polyacrylamide gels (Invitrogen, Inc, Carlsbad, Calif.) under denaturing, reducing conditions. Separated polypeptides were transferred from the gel onto polyvinylidene fluoride (PVDF) membranes (Invitrogen, Inc, Carlsbad, Calif.) by Western blotting using a Trans-Blot® SD semi-dry electrophoretic transfer cell apparatus (Bio-Rad Laboratories, Hercules, Calif.). PVDF membranes were blocked by incubating at room temperature for 2 hours in a solution containing 25 mM Tris-Buffered Saline (25 mM 2-amino-2-hydroxymethyl-1,3-propanediol hydrochloric acid (Tris-HCl) (pH 7.4), 137 mM sodium chloride, 2.7 mM potassium chloride), 0.1% TWEEN-20®, polyoxyethylene (20) sorbitan monolaureate, 2% bovine serum albumin, 5% nonfat dry milk. Blocked membranes were incubated at 4° C. for overnight in Tris-Buffered Saline TWEEN-20® (25 mM Tris-Buffered Saline, 0.1% TWEEN-20®, polyoxyethylene (20) sorbitan monolaureate) containing appropriate primary antibodies as a probe. Primary antibody probed blots were washed three times for 15 minutes each time in Tris-Buffered Saline TWEEN-20®. Washed membranes were incubated at room temperature for 2 hours in Tris-Buffered Saline TWEEN-20® containing an appropriate immunoglobulin G antibody conjugated to horseradish peroxidase as a secondary antibody. Secondary antibody-probed blots were washed three times for 15 minutes each time in Tris-Buffered Saline TWEEN-20®. Signal detection of the labeled modified Clostridial toxin were visualized using the ECL Plus™ Western Blot Detection System (Amersham Biosciences, Piscataway, N.J.) and were imaged with a Typhoon 9410 Variable Mode Imager (GE Healthcare, Piscataway, N.J.) for quantification of modified Clostridial toxin expression levels.

### Example 4

### Activation of Modified Clostridial Toxin with Integrated Protease Cleavage Site-Binding Domain

The following example illustrates methods useful for activating any of the modified Clostridial toxins with an integrated protease cleavage site-binding domain disclosed in the present specification by converting the single-chain form of such toxins into the di-chain form.

To activate a modified Clostridial toxin disclosed in the present specification, a reaction mixture was set up by adding 2.5 to 10 units of AcTEV (Invitrogen, Inc., Carlsbad, Calif.) to a 50 mM Tris-HCl (pH 8.0) solution containing 1.0 μg of a purified modified Clostridial toxin, such as, e.g., as described in Example 3. This reaction mixture was incubated at 23-30° C. for 60-180 minutes. To analyze the conversion of the single-chain form into its di-chain form small aliquots of the reaction mixture, with and without DTT, were separated by MOPS polyacrylamide gel electrophoresis using NuPAGE® Novex 4-12% Bis-Tris precast polyacrylamide gels (Invitrogen, Inc, Carlsbad, Calif.) under denaturing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.) for quantification of the single-chain and di-chain forms of the modified Clostridial toxin. The size and amount of modified Clostridial toxin form was determined by comparison to MagicMark™ protein molecular weight standards (Invitrogen, Inc, Carlsbad, Calif.).

The results indicate that following TEV nicking in the integrated protease cleavage-site binding domain of a modified Clostrifidial toxin, two bands of approximately 50 kDa each, corresponding to the di-chain form of the modified toxin, were detected under reducing conditions. Moreover, when the same sample was run under non-reducing conditions, the two approximately 50 kDa bands disappeared and a new band of approximately 100 kDa was observed. Taken together, these observations indicate that the two approximately 50 kDa bands seen under reducing conditions correspond to the Clostridial toxin enzymatic domain and the Clostridial toxin translocation domain with the targeting moiety attached to its amino terminus.

### Example 5

### Purification of Activated Modified Clostridial Toxin with Integrated Protease Cleavage Site-Binding Domain

The following example illustrates methods useful for purifying and quantifying the di-chain form of modified Clostridial toxins disclosed in the present specification after activation with TEV.

To purify an activated modified Clostridial toxin disclosed in the present specification, a reaction mixture containing a modified Clostridial toxin treated with a TEV protease, such as, e.g., as described in Example 4, was subjected to an anion exchange chromatography purification procedures to remove the TEV protease and recover the di-chain modified Clostridial toxin. The reaction mixture was loaded onto a 1.0 mL UNO-Q1™ Anion exchange column (Bio-Rad Laboratories, Hercules, Calif.) equilibrated with 50 mM Tris-HCl (pH 8.0) at a flow rate of 1.0 mL/min. Bound proteins were eluted by a NaCl gradient using an elution buffer comprising 50 mM Tris-HCL (pH 8.0) and 1M NaCl as follows: 3 mL of 7% elution buffer at a flow rate of 1.0 mL/min, 6 mL of 12% elution buffer at a flow rate of 1.0 mL/min, and 10 mL of 12% to 100% elution buffer at a flow rate of 1.0 mL/min. Elution of material from the column was detected with a QuadTec UV-Vis detector at 214 nm, 260 nm, and 280 nm and all peaks absorbing at or above 0.01 AU at 180 nm were collected in 1.0 mL fractions. Selected fractions were added to 2×LDS Sample Buffer (Invitrogen, Inc, Carlsbad, Calif.) with and without DTT and separated by MOPS polyacrylamide gel electrophoresis using NuPAGE® Novex 4-12% Bis-Tris precast polyacrylamide gels (Invitrogen, Inc, Carlsbad, Calif.) under denaturing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.) for quantification of the purified activated modified Clostridial toxin. Peak fractions were pooled, 5% PEG-400 was added, and the purified samples were frozen in liquid nitrogen and stored at −80° C.

### Example 6

### Construction of a Modified Clostridial Toxin Comprising an Integrated TEV Protease Cleavage Site-Galanin Binding Domain

The following example illustrates methods useful for constructing a modified Clostridial toxin comprising a di-chain loop comprising an integrated TEV protease cleavage site Galanin binding domain disclosed in the present specification.

To construct a modified Clostridial toxin comprising an integrated TEV protease cleavage site Galanin binding domain, a re-targeted toxin comprising a nociceptin targeting moiety was modified to replace the existing enterokinase cleavage site and nociceptin targeting moiety with an integrated protease cleavage site-Galanin binding domain. Examples of re-targeted toxins comprising an enterokinase cleavage site and nociceptin targeting moiety are disclosed in, e.g., Steward, U.S. patent application Ser. No. 12/192,900, supra, (2008); Foster, U.S. patent application Ser. No. 11/792,210, supra, (2007); Foster, U.S. patent application Ser. No. 11/791,979, supra, (2007); Dolly, U.S. Pat. No. 7,419,676, supra, (2008), each of which is hereby incorporated by reference in its entirety. For example, a 7.89-kb expression construct comprising polynucleotide molecule of SEQ ID NO: 148 was digested with EcoRI and XbaI, excising the 260 by polynucleotide molecule encoding the enterokinase cleavage site and the nociceptin targeting moiety and the resulting 7.63 kb EcoRI-XbaI fragment was purified using a gel-purification procedure. A 311 by EcoRI-XbaI fragment (SEQ ID NO: 187) encoding the integrated protease cleavage site-Galanin of SEQ ID NO: 188 was subcloned into the purified 7.63 kb EcoRI-XbaI fragment using a T4 DNA ligase procedure. The ligation mixture was transformed into electro-competent E. coli BL21(DE3) cells (Edge Biosystems, Gaithersburg, Md.) using an electroporation method, and the cells were plated on 1.5% Luria-Bertani agar plates (pH 7.0) containing 50 μg/mL of kanamycin, and were placed in a 37° C. incubator for overnight growth. Bacteria containing expression constructs were identified as kanamycin resistant colonies. Candidate constructs were isolated using an alkaline lysis plasmid mini-preparation procedure and analyzed by restriction endonuclease digest mapping to determine the presence and orientation of the insert and by DNA sequencing. This cloning strategy yielded a pET29 expression construct comprising the polynucleotide molecule of SEQ ID NO: 189 encoding the BoNT/A-IPCS-Galanin of SEQ ID NO: 190.

Alternatively, a polynucleotide molecule based on BoNT/A-IPCS-Galanin (SEQ ID NO: 190) comprising the IPCS-Galanin of SEQ ID NO: 188 can be synthesized using standard procedures (BlueHeron® Biotechnology, Bothell, Wash.). Oligonucleotides of 20 to 50 bases in length are synthesized using standard phosphoramidite synthesis. These oligonucleotides will be hybridized into double stranded duplexes that are ligated together to assemble the full-length polynucleotide molecule. This polynucleotide molecule will be cloned using standard molecular biology methods into a pUCBHB1 vector at the SmaI site to generate pUCBHB1/BoNT/A-AP4A-Galanin. The synthesized polynucleotide molecule is verified by sequencing using Big Dye Terminator™ Chemistry 3.1 (Applied Biosystems, Foster City, Calif.) and an ABI 3100 sequencer (Applied Biosystems, Foster City, Calif.). If desired, an expression optimized polynucleotide molecule based on BoNT/A-IPCS-Galanin (SEQ ID NO: 190) can be synthesized in order to improve expression in an Escherichia coli strain. The polynucleotide molecule encoding the BoNT/A-IPCS-Galanin can be modified to 1) contain synonymous codons typically present in native polynucleotide molecules of an Escherichia coli strain; 2) contain a G+C content that more closely matches the average G+C content of native polynucleotide molecules found in an Escherichia coli strain; 3) reduce polymononucleotide regions found within the polynucleotide molecule; and/or 4) eliminate internal regulatory or structural sites found within the polynucleotide molecule, see, e.g., Lance E. Steward et al., Optimizing Expression of Active Botulinum Toxin Type A, U.S. Patent Publication 2008/0057575 (Mar. 6, 2008); and Lance E. Steward et al., Optimizing Expression of Active Botulinum Toxin Type E, U.S. Patent Publication 2008/0138893 (Jun. 12, 2008). Once sequence optimization is complete, oligonucleotides of 20 to 50 bases in length are synthesized using standard phosphoramidite synthesis. These oligonucleotides are hybridized into double stranded duplexes that are ligated together to assemble the full-length polynucleotide molecule. This polynucleotide molecule is cloned using standard molecular biology methods into a pUCBHB1 vector at the SmaI site to generate pUCBHB1/BoNT/A-IPCS-Galanin. The synthesized polynucleotide molecule is verified by DNA sequencing. If so desired, expression optimization to a different organism, such as, e.g., a yeast strain, an insect cell-line or a mammalian cell line, can be done, see, e.g., Steward, U.S. Patent Publication 2008/0057575, supra, (2008); and Steward, U.S. Patent Publication 2008/0138893, supra, (2008).

To construct pET29/BoNT/A-IPCS-Galanin, a pUCBHB1/BoNT/A-IPCS-Galanin construct was digested with restriction endonucleases that 1) excised the polynucleotide molecule encoding the open reading frame of BoNT/A-IPCS-Galanin; and 2) enabled this polynucleotide molecule to be operably-linked to a pET29 vector (EMD Biosciences-Novagen, Madison, Wis.). This insert was subcloned using a T4 DNA ligase procedure into a pET29 vector that was digested with appropriate restriction endonucleases to yield pET29/BoNT/A-IPCS-Galanin. The ligation mixture was transformed into electro-competent E. coli BL21(DE3) cells (Edge Biosystems, Gaitherburg, Md.) using an electroporation method, and the cells were plated on 1.5% Luria-Bertani agar plates (pH 7.0) containing 50 μg/mL of kanamycin, and placed in a 37° C. incubator for overnight growth. Bacteria containing expression constructs were identified as kanamycin resistant colonies. Candidate constructs were isolated using an alkaline lysis plasmid mini-preparation procedure and were analyzed by restriction endonuclease digest mapping to determine the presence and orientation of the insert. This cloning strategy yielded a pET29 expression construct comprising the polynucleotide molecule encoding the BoNT/A-IPCS-Galanin.

### Example 7

### Expression of Modified Clostridial Toxin Comprising an Integrated TEV Protease Cleavage Site-Galanin Binding Domain

The following example illustrates a procedure useful for expressing a modified Clostridial toxin comprising an integrated TEV protease cleavage site-Galanin binding domain in a bacterial cell.

To express a modified Clostridial toxin disclosed comprising an integrated TEV protease cleavage site-Galanin binding domain, an expression construct, such as, e.g., as described in Example 6, was transformed into electro-competent E. coli BL21 (DE3) Acella® cells (Edge Biosystems, Gaithersburg, Md.) using an electroporation method. The cells were then plated onto 1.5% Luria-Bertani agar plates (pH 7.0) containing 50 μg/mL of kanamycin and placed in a 37° C. incubator for overnight growth. Kanamycin-resistant colonies of transformed E. coli containing the expression construct were used to inoculate a baffled flask containing 3.0 mL of PA-0.5G media containing 50 μg/mL of kanamycin which was then placed in a 37° C. incubator, shaking at 250 rpm, for overnight growth. The resulting overnight starter culture was used to inoculate 250 mL ZYP-5052 autoinducing media containing 50 μg/mL of kanamycin. These cultures were grown in a 37° C. incubator shaking at 250 rpm for approximately 3.5 hours and were then transferred to a 22° C. incubator shaking at 250 rpm for an additional incubation of 16-18 hours. Cells were harvested by centrifugation (4,000 rpm at 4° C. for 20-30 minutes) and were used immediately, or stored dry at −80° C. until needed.

### Example 8

### Purification of Modified Clostridial Toxin Comprising an Integrated TEV Protease Cleavage Site-Galanin Binding Domain

The following example illustrates methods useful for purifying and quantifying a modified Clostridial toxin comprising an integrated TEV protease cleavage site-Galanin binding domain.

To lyse cell pellets containing a modified Clostridial toxin comprising an integrated TEV protease cleavage site-Galanin binding domain, a cell pellet, such as, e.g., as described in Example 7, was resuspended in a lysis buffer containing BUGBUSTER® Protein Extraction Reagent (EMD Biosciences-Novagen, Madison, Wis.); 1× Protease Inhibitor Cocktail Set III (EMD Biosciences-Calbiochem, San Diego Calif.); 25 unit/mL Benzonase nuclease (EMD Biosciences-Novagen, Madison, Wis.); and 1,000 units/mL rLysozyme (EMD Biosciences-Novagen, Madison, Wis.). The cell suspension was incubated at room temperature on a platform rocker for 20 minutes, incubated on ice for 15 minutes to precipitate detergent, than centrifuged at 30,500 rcf for 30 minutes at 4° C. to remove insoluble debris. The clarified supernatant was transferred to a new tube and was used immediately for IMAC purification, or stored dry at 4° C. until needed.

To purify a modified Clostridial toxin comprising an integrated TEV protease cleavage site-Galanin binding domain using immobilized metal affinity chromatography (IMAC), the clarified supernatant was mixed with 2.5-5.0 mL of TALON™ SuperFlow Co²⁺ affinity resin (BD Biosciences-Clontech, Palo Alto, Calif.) equilibrated with IMAC Wash Buffer (25 mM N-(2-hydroxyethyl) piperazine-N′-(2-ethanesulfonic acid) (HEPES), pH 8.0; 500 mM sodium chloride; 10 mM imidazole; 10% (v/v) glycerol). The clarified supernatant-resin mixture was incubated on a platform rocker for 60 minutes at 4° C. The clarified supernatant-resin mixture was then transferred to a disposable polypropylene column support (Thomas Instruments Co., Philadelphia, Pa.) and attached to a vacuum manifold. The column was washed twice with five column volumes of IMAC Wash Buffer. The modified Clostridial toxin was eluted with 2 column volumes of IMAC Elution Buffer (25 mM N-(2-hydroxyethyl) piperazine-N′-(2-ethanesulfonic acid) (HEPES), pH 8.0; 500 mM sodium chloride; 500 mM imidazole; 10% (v/v) glycerol) and collected in approximately 1 mL fractions. The amount of modified Clostridial toxin contained in each elution fraction was determined by a Bradford dye assay. In this procedure, a 10 μL aliquot of each 1.0 mL fraction was combined with 200 μL of Bio-Rad Protein Reagent (Bio-Rad Laboratories, Hercules, Calif.), diluted 1 to 4 with deionized, distilled water, and the intensity of the colorimetric signal was measured using a spectrophotometer. The fractions with the strongest signal were considered the elution peak and were combined together and dialyzed to adjust the solution for subsequent procedures. Buffer exchange of IMAC-purified modified Clostridial toxin was accomplished by dialysis at 4° C. in a FASTDIALYZER® (Harvard Apparatus) fitted with 25 kD MWCO membranes (Harvard Apparatus). The protein samples were exchanged into the appropriate Desalting Buffer (50 mM Tris-HCl (pH 8.0) to be used in the subsequent activation step. The FASTDIALYZER® was placed in 1 L Desalting Buffer with constant stirring and incubated overnight at 4° C.

Expression of a modified Clostridial toxin comprising an integrated TEV protease cleavage site-Galanin binding domain was analyzed by polyacrylamide gel electrophoresis. Samples of modified Clostridial toxin, purified using the procedure described above, are added to 2×LDS Sample Buffer (Invitrogen, Inc, Carlsbad, Calif.) with and without DTT and separated by MOPS polyacrylamide gel electrophoresis using NuPAGE® Novex 4-12% Bis-Tris precast polyacrylamide gels (Invitrogen, Inc, Carlsbad, Calif.) under denaturing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.). To quantify modified Clostridial toxin yield, varying amounts of purified modified Clostridial toxin samples were added to 2×LDS Sample Buffer (Invitrogen, Inc, Carlsbad, Calif.) without DTT and were separated on by MOPS polyacrylamide gel electrophoresis using NuPAGE® Novex 4-12% Bis-Tris precast polyacrylamide gels (Invitrogen, Inc, Carlsbad, Calif.) under non-reducing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.). Following imaging, a reference curve is plotted for the BSA standards and the toxin quantities interpolated from this curve. The size of modified Clostridial toxin was determined by comparison to MagicMark™ protein molecular weight standards (Invitrogen, Inc, Carlsbad, Calif.).

### Example 9

### Activation of Modified Clostridial Toxin Comprising an Integrated TEV Protease Cleavage Site-Galanin Binding Domain

The following example illustrates methods useful for activating the modified Clostridial toxin with an integrated protease cleavage site-Galanin binding domain by converting the single-chain form of the protein into the di-chain form.

To activate a modified Clostridial toxin with an integrated protease cleavage site-Galanin binding domain, a reaction mixture was set up by adding 2.5 to 10 units of AcTEV (Invitrogen, Inc., Carlsbad, Calif.) to a 50 mM Tris-HCl (pH 8.0) solution containing 1.0 μg of a purified modified Clostridial toxin, such as, e.g., as described in Example 8. This reaction mixture was incubated at 23-30° C. for 60-180 minutes. To analyze the conversion of the single-chain form into its di-chain form small aliquots of the reaction mixture, with and without DTT, were separated by MOPS polyacrylamide gel electrophoresis using NuPAGE® Novex 4-12% Bis-Tris precast polyacrylamide gels (Invitrogen, Inc, Carlsbad, Calif.) under denaturing conditions. Gels were stained with SYPRO® Ruby (Bio-Rad Laboratories, Hercules, Calif.) and the separated polypeptides imaged using a Fluor-S MAX MultiImager (Bio-Rad Laboratories, Hercules, Calif.) for quantification of the single-chain and di-chain forms of the modified Clostridial toxin. The size of modified Clostridial toxin was determined by comparison to MagicMark™ protein molecular weight standards (Invitrogen, Inc, Carlsbad, Calif.).

The results indicate that following TEV nicking in the integrated protease cleavage-site binding domain of a modified Clostrifidial toxin, two bands of approximately 50 kDa each, corresponding to the di-chain form of the modified toxin, were detected under reducing conditions. Moreover, when the same sample was run under non-reducing conditions, the two approximately 50 kDa bands disappeared and a new band of approximately 100 kDa was observed. Taken together, these observations indicate that the two approximately 50 kDa bands seen under reducing conditions correspond to the Clostridial toxin enzymatic domain and the Clostridial toxin translocation domain with the Galanin moiety attached to its amino terminus.

### Example 10

### Purification of Activated Modified Clostridial Toxin Comprising an Integrated TEV Protease Cleavage Site-Galanin Binding Domain

The following example illustrates methods useful for purifying and quantifying the di-chain form of a modified Clostridial toxin with an integrated protease cleavage site-Galanin binding domain, after activation with TEV.

To purify an activated modified Clostridial toxin with an integrated protease cleavage site-Galanin binding domain, a reaction mixture containing a modified Clostridial toxin treated with a TEV protease, such as, e.g., as described in Example 9, was subjected to an anion exchange chromatography purification procedures to remove the TEV protease and recover the di-chain modified Clostridial toxin. The reaction mixture was loaded onto a 1.0 mL UNO-Q1™ Anion exchange column (Bio-Rad Laboratories, Hercules, Calif.) equilibrated with 50 mM Tris-HCl (pH 8.0) at a flow rate of 1.0 mL/min. Bound proteins were eluted by a NaCl gradient using an elution buffer comprising 50 mM Tris-HCL (pH 8.0) and 1M NaCl as follows: 3 mL of 7% elution buffer at a flow rate of 1.0 mL/min, 6 mL of 12% elution buffer at a flow rate of 1.0 mL/min, and 10 mL of 12% to 100% elution buffer at a flow rate of 1.0 mL/min. Elution of material from the column was detected with a QuadTec UV-Vis detector at 214 nm, 260 nm, and 280 nm and all peaks absorbing at or above 0.01 AU at 180 nm were collected in 1.0 mL fractions. Selected fractions were added to 2×LDS Sample Buffer (Invitrogen, Inc, Carlsbad, Calif.) with and without DTT and separated by MOPS polyacrylamide gel electrophoresis using NuPAGE® Novex 4-12% Bis-Tris precast polyacrylamide gels (Invitrogen, Inc, Carlsbad, Calif.) under denaturing conditions. Gels were stained with SYPRO® Ruby (Bio-Rad Laboratories, Hercules, Calif.) and the separated polypeptides were imaged using a Fluor-S MAX MultiImager (Bio-Rad Laboratories, Hercules, Calif.) for quantification of the purified activated modified Clostridial toxin. Peak fractions were pooled, 5% PEG-400 was added, and the purified samples were frozen in liquid nitrogen and stored at −80° C.

Although aspects of the present invention have been described with reference to the disclosed embodiments, one skilled in the art will readily appreciate that the specific examples disclosed are only illustrative of these aspects and in no way limit the present invention. Various modifications can be made without departing from the spirit of the present invention.

Although aspects of the present invention have been described with reference to the disclosed embodiments, one skilled in the art will readily appreciate that the specific examples disclosed are only illustrative of these aspects and in no way limit the present invention. Various modifications can be made without departing from the spirit of the present invention.

What is claimed:

1. A single-chain modified Clostridial toxin comprising:
a) a Clostridial toxin enzymatic domain capable of executing an enzymatic target modification step of a Clostridial toxin intoxication process;
b) a Clostridial toxin translocation domain capable of executing a translocation step of a Clostridial toxin intoxication process; and
c) an integrated protease cleavage site-binding domain comprising a P portion of a protease cleavage site including the P₁ site of the scissile bond and a binding domain, the P₁ site of the P portion of the protease cleavage site abutting the amino-end of the binding domain thereby creating an integrated protease cleavage site;
wherein cleavage of the integrated protease cleavage site-binding domain converts the single-chain modified Clostridial toxin into a di-chain form and produces a binding domain with an amino-terminus capable of binding to its cognate receptor.

2. The modified Clostridial toxin of claim 1, wherein the modified Clostridial toxin comprises a linear amino-to-carboxyl single polypeptide order of 1) the Clostridial toxin enzymatic domain, the Clostridial toxin translocation domain, and the integrated protease cleavage site-binding domain, 2) the Clostridial toxin enzymatic domain, the integrated protease cleavage site-binding domain, and the Clostridial toxin translocation domain, 3) the integrated protease cleavage site-binding domain, the Clostridial toxin translocation domain, and the Clostridial toxin enzymatic domain, 4) the integrated protease cleavage site-binding domain, the Clostridial toxin enzymatic domain, and the Clostridial toxin translocation domain, or 5) the Clostridial toxin translocation domain, integrated protease cleavage site-binding domain, and the Clostridial toxin enzymatic domain.

3. The modified Clostridial toxin of claim 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

4. The modified Clostridial toxin of claim 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

5. The modified Clostridial toxin of claim 1, wherein the integrated protease cleavage site-binding domain is any one of SEQ ID NO: 4 to SEQ ID NO: 118.

6. The modified Clostridial toxin of claim 1, wherein the P portion of a protease cleavage site including the P₁ site of the scissile bond is SEQ ID NO: 121, SEQ ID NO: 127, or SEQ ID NO: 130.

7. The modified Clostridial toxin of claim 1, wherein the binding domain is an opioid peptide.

8. The modified Clostridial toxin of claim 7, wherein the opioid peptide is an enkephalin, a BAM22 peptide, an endomorphin, an endorphin, a dynorphin, a nociceptin or a rimorphin.

9. The modified Clostridial toxin of claim 1, wherein the binding domain is a PAR ligand.

10. The modified Clostridial toxin of claim 9, wherein the PAR ligand is a PAR1, a PAR2, a PAR3, or a PAR4.

11. A pharmaceutical composition comprising a di-chain from a single-chain modified Clostridial toxin of claim 1 and a pharmaceutically acceptable carrier, a pharmaceutically acceptable component, or both a pharmaceutically acceptable carrier and a pharmaceutically acceptable component.

12. A polynucleotide molecule encoding a modified Clostridial toxin according to claim 1.

13. The polynucleotide molecule according to claim 12, wherein the polynucleotide molecule further comprises an expression vector.

14. A method of producing a modified Clostridial toxin comprising the steps of:
a) introducing into a cell a polynucleotide molecule of claim 13; and
b) expressing the polynucleotide molecule.


SEQUENCE LISTING (198 sequences)

SEQ ID NO: 1 - protein, 10 aa, Artificial Sequence
  Human Rhinovirus 3C protease cleavage site consensus sequence
XXLFQGPXXX

SEQ ID NO: 2 - protein, 10 aa, Artificial Sequence
  Factor Xa cleavage site consensus sequence
XIXGRXXXXX

SEQ ID NO: 3 - protein, 10 aa, Artificial Sequence
  Enterokinase cleavage site consensus sequence
DDDDKXXXXX

SEQ ID NO: 4 - protein, 11 aa, Artificial Sequence
  Integrated protease cleavage site-binding domain (enkephalin)
EXXYXQYGGFL

SEQ ID NO: 5 - protein, 11 aa, Artificial Sequence
  Integrated protease cleavage site-binding domain (enkephalin)
EXXYXQYGGFM

SEQ ID NO: 6 - protein, 14 aa, Artificial Sequence
  Integrated protease cleavage site-binding domain (enkephalin)
EXXYXQYGGFMRGL

SEQ ID NO: 7 - protein, 13 aa, Artificial Sequence
  Integrated protease cleavage site-binding domain (enkephalin)
EXXYXQYGGFMRF

SEQ ID NO: 8 - protein, 18 aa, Artificial Sequence
  Integrated protease cleavage site-binding domain (BAM22)
EXXYXQYGGFMRRVGRPD

SEQ ID NO: 9 - protein, 18 aa, Artificial Sequence
  Integrated protease cleavage site-binding domain (BAM22)
EXXYXQYGGFMRRVGRPD

SEQ ID NO: 10 - protein, 22 aa, Artificial Sequence
  Integrated protease cleavage site-binding domain (BAM22)
EXXYXQRVGRPEWWMDYQKRYG

SEQ ID NO: 11 - protein, 22 aa, Artificial Sequence
  Integrated protease cleavage site-binding domain (BAM22)
EXXYXQRVGRPEWWLDYQKRTG

SEQ ID NO: 12 - protein, 22 aa, Artificial Sequence
  Integrated protease cleavage site-binding domain (BAM22)
EXXYXQRVGRPEWWQDYQKRYG

SEQ ID NO: 13 - protein, 22 aa, Artificial Sequence
  Integrated protease cleavage site-binding domain (BAM22)
EXXYXQRVGRPEWWEDYQKRYG

SEQ ID NO: 14 - protein, 22 aa, Artificial Sequence
  Integrated protease cleavage site-binding domain (BAM22)
EXXYXQRVGRPEWKLDNQKRYG

SEQ ID NO: 15 - protein, 21 aa, Artificial Sequence
  Integrated protease cleavage site-binding domain (BAM22)
EXXYXQRVGRPDWWQESKRYG

SEQ ID NO: 16 - protein, 20 aa, Artificial Sequence
  Integrated protease cleavage site-binding domain (BAM22)
EXXYXQGRPEWWMDYQKRYG

SEQ ID NO: 17 - protein, 20 aa, Artificial Sequence
  Integrated protease cleavage site-binding domain (BAM22)
EXXYXQGRPEWWLDYQKRTG

SEQ ID NO: 18 - protein, 20 aa, Artificial Sequence
  Integrated protease cleavage site-binding domain (BAM22)
EXXYXQGRPEWWEDYQKRYG

SEQ ID NO: 19 - protein, 20 aa, Artificial Sequence
  Integrated protease cleavage site-binding domain (BAM22)
EXXYXQGRPEWWEDYQKRYG

SEQ ID NO: 20 - protein, 20 aa, Artificial Sequence
  Integrated protease cleavage site-binding domain (BAM22)
EXXYXQGRPEWKLDNQKRYG

SEQ ID NO: 21 - protein, 19 aa, Artificial Sequence
  Integrated protease cleavage site-binding domain (BAM22)
EXXYXQGRPDWWQESKRYG

SEQ ID NO: 22 - protein, 28 aa, Artificial Sequence
  Integrated protease cleavage site-binding domain (BAM22)
EXXYXQYGGFMRRVGRPEWWMDYQKRYG

SEQ ID NO: 23 - protein, 28 aa, Artificial Sequence
  Integrated protease cleavage site-binding domain (BAM22)
EXXYXQYGGFMRRVGRPEWWLDYQKRTG

SEQ ID NO: 24 - protein, 28 aa, Artificial Sequence
  Integrated protease cleavage site-binding domain (BAM22)
EXXYXQYGGFMRRVGRPEWWQDYQKRYG

SEQ ID NO: 25 - protein, 28 aa, Artificial Sequence
  Integrated protease cleavage site-binding domain (BAM22)
EXXYXQYGGFMRRVGRPEWWEDYQKRYG

SEQ ID NO: 26 - protein, 28 aa, Artificial Sequence
  Integrated protease cleavage site-binding domain (BAM22)
EXXYXQYGGFMRRVGRPEWKLDNQKRYG

SEQ ID NO: 27 - protein, 27 aa, Artificial Sequence
  Integrated protease cleavage site-binding domain (BAM22)
EXXYXQYGGFMRRVGRPDWWQESKRYG

SEQ ID NO: 28 - protein, 10 aa, Artificial Sequence
  Integrated protease cleavage site-binding domain (Endomorphin)
EXXYXQYPYF

SEQ ID NO: 29 - protein, 10 aa, Artificial Sequence
  Integrated protease cleavage site-binding domain (Endomorphin)
EXXYXQYPFF

SEQ ID NO: 30 - protein, 22 aa, Artificial Sequence
  Integrated protease cleavage site-binding domain (Endorphin)
EXXYXQYGGFMTSEKSQTPLVT

SEQ ID NO: 31 - protein, 16 aa, Artificial Sequence
  Integrated protease cleavage site-binding domain (Endorphin)
EXXYXQYGGFLRKYPK

SEQ ID NO: 32 - protein, 37 aa, Artificial Sequence
  Integrated protease cleavage site-binding domain (Endorphin)
EXXYXQYGGFMTSEKSQTPLVTLFKNAIIKNAYKKGE

SEQ ID NO: 33 - protein, 37 aa, Artificial Sequence
  Integrated protease cleavage site-binding domain (Endorphin)
EXXYXQYGGFMSSEKSQTPLVTLFKNAIIKNAHKKGQ

SEQ ID NO: 34 - protein, 15 aa, Artificial Sequence
  Integrated protease cleavage site-binding domain (Endorphin)
EXXYXQYGGFLRKYP

SEQ ID NO: 35 - protein, 23 aa, Artificial Sequence
  Integrated protease cleavage site-binding domain (Endorphin)
EXXYXQYGGFMTSEKSQTPLVTL

SEQ ID NO: 36 - protein, 23 aa, Artificial Sequence
  Integrated protease cleavage site-binding domain (Dynorphin)
EXXYXQYGGFLRRIRPKLKWDNQ

SEQ ID NO: 37 - protein, 19 aa, Artificial Sequence
  Integrated protease cleavage site-binding domain (Dynorphin)
EXXYXQYGGFLRRIRPKLK

SEQ ID NO: 38 - protein, 22 aa, Artificial Sequence
  Integrated protease cleavage site-binding domain (Dynorphin)
EXXYXQGGFLRRIRPKLKWDNQ

SEQ ID NO: 39 - protein, 18 aa, Artificial Sequence
  Integrated protease cleavage site-binding domain (Dynorphin)
EXXYXQGGFLRRIRPKLK

SEQ ID NO: 40 - protein, 23 aa, Artificial Sequence
  Integrated protease cleavage site-binding domain (Dynorphin)
EXXYXQYGGFLRRIRPKLRWDNQ

SEQ ID NO: 41 - protein, 19 aa, Artificial Sequence
  Integrated protease cleavage site-binding domain (Dynorphin)
EXXYXQYGGFLRRIRPKLR

SEQ ID NO: 42 - protein, 23 aa, Artificial Sequence
  Integrated protease cleavage site-binding domain (Dynorphin)
EXXYXQYGGFLRRIRPRLRWDNQ

SEQ ID NO: 43 - protein, 19 aa, Artificial Sequence
  Integrated protease cleavage site-binding domain (Dynorphin)
EXXYXQYGGFLRRIRPRLR

SEQ ID NO: 44 - protein, 23 aa, Artificial Sequence
  Integrated protease cleavage site-binding domain (Dynorphin)
EXXYXQYGGFMRRIRPKLRWDNQ

SEQ ID NO: 45 - protein, 19 aa, Artificial Sequence
  Integrated protease cleavage site-binding domain (Dynorphin)
EXXYXQYGGFMRRIRPKLR

SEQ ID NO: 46 - protein, 23 aa, Artificial Sequence
  Integrated protease cleavage site-binding domain (Dynorphin)
EXXYXQYGGFMRRIRPKIRWDNQ

SEQ ID NO: 47 - protein, 19 aa, Artificial Sequence
  Integrated protease cleavage site-binding domain (Dynorphin)
EXXYXQYGGFMRRIRPKIR

SEQ ID NO: 48 - protein, 23 aa, Artificial Sequence
  Integrated protease cleavage site-binding domain (Dynorphin)
EXXYXQYGGFMRRIRPKLKWDSQ

SEQ ID NO: 49 - protein, 19 aa, Artificial Sequence
  Integrated protease cleavage site-binding domain (Dynorphin)
EXXYXQYGGFMRRIRPKLK

SEQ ID NO: 50 - protein, 15 aa, Artificial Sequence
  Integrated protease cleavage site-binding domain (Dynorphin)
EXXYXQYGGFLRRIR

SEQ ID NO: 51 - protein, 15 aa, Artificial Sequence
  Integrated protease cleavage site-binding domain (Dynorphin)
EXXYXQYGGFMRRIR

SEQ ID NO: 52 - protein, 35 aa, Artificial Sequence
  Integrated protease cleavage site-binding domain (Dynorphin)
EXXYXQYGGFLRRQFKVVTRSQEDPNAYSGELFDA

SEQ ID NO: 53 - protein, 34 aa, Artificial Sequence
  Integrated protease cleavage site-binding domain (Dynorphin)
EXXYXQYGGFLRRQFKVVTRSQENPNTYSEDLDV

SEQ ID NO: 54 - protein, 34 aa, Artificial Sequence
  Integrated protease cleavage site-binding domain (Dynorphin)
EXXYXQYGGFLRRQFKVVTRSQESPNTYSEDLDV

SEQ ID NO: 55 - protein, 35 aa, Artificial Sequence
  Integrated protease cleavage site-binding domain (Dynorphin)
EXXYXQYGGFLRRQFKVVTRSQEDPNAYSEEFFDV

SEQ ID NO: 56 - protein, 35 aa, Artificial Sequence
  Integrated protease cleavage site-binding domain (Dynorphin)
EXXYXQYGGFLRRQFKVVTRSQEDPNAYYEELFDV

SEQ ID NO: 57 - protein, 35 aa, Artificial Sequence
  Integrated protease cleavage site-binding domain (Dynorphin)
EXXYXQYGGFLRRQFKVVTRSQEDPNAYSGELLDG

SEQ ID NO: 58 - protein, 35 aa, Artificial Sequence
  Integrated protease cleavage site-binding domain (Dynorphin)
EXXYXQYGGFLRRQFKVVTRSQEDPSAYYEELFDV

SEQ ID NO: 59 - protein, 35 aa, Artificial Sequence
  Integrated protease cleavage site-binding domain (Dynorphin)
EXXYXQYGGFLRRQFKVTTRSEEDPSTFSGELSNL

SEQ ID NO: 60 - protein, 35 aa, Artificial Sequence
  Integrated protease cleavage site-binding domain (Dynorphin)
EXXYXQYGGFLRRQFKVTTRSEEEPGSFSGEISNL

SEQ ID NO: 61 - protein, 35 aa, Artificial Sequence
  Integrated protease cleavage site-binding domain (Dynorphin)
EXXYXQYGGFLRRQFKVNARSEEDPTMFSDELSYL

SEQ ID NO: 62 - protein, 35 aa, Artificial Sequence
  Integrated protease cleavage site-binding domain (Dynorphin)
EXXYXQYGGFLRRQFKVNARSEEDPTMFSGELSYL

SEQ ID NO: 63 - protein, 35 aa, Artificial Sequence
  Integrated protease cleavage site-binding domain (Dynorphin)
EXXYXQYGGFLRRHFKISVRSDEEPSSYSDEVLEL

SEQ ID NO: 64 - protein, 35 aa, Artificial Sequence
  Integrated protease cleavage site-binding domain (Dynorphin)
EXXYXQYGGFLRRHFKITVRSDEDPSPYLDEFSDL

SEQ ID NO: 65 - protein, 33 aa, Artificial Sequence
  Integrated protease cleavage site-binding domain (Dynorphin)
EXXYXQYGGFLRRHFKISVRSDEEPSSYEDYAL

SEQ ID NO: 66 - protein, 33 aa, Artificial Sequence
  Integrated protease cleavage site-binding domain (Dynorphin)
EXXYXQYGGFLRRHFKISVRSDEEPGSYDVIGL

SEQ ID NO: 67 - protein, 33 aa, Artificial Sequence
  Integrated protease cleavage site-binding domain (Dynorphin)
EXXYXQYGGFLRRHYKLSVRSDEEPSSYDDFGL

SEQ ID NO: 68 - protein, 13 aa, Artificial Sequence
  Integrated protease cleavage site-binding domain (Dynorphin)
EXXYXQYGGFLRR

SEQ ID NO: 69 - protein, 19 aa, Artificial Sequence
  Integrated protease cleavage site-binding domain (Rimorphin)
EXXYXQYGGFLRRQFKVVT

SEQ ID NO: 70 - protein, 19 aa, Artificial Sequence
  Integrated protease cleavage site-binding domain (Rimorphin)
EXXYXQYGGFLRRQFKVTT

SEQ ID NO: 71 - protein, 19 aa, Artificial Sequence
  Integrated protease cleavage site-binding domain (Rimorphin)
EXXYXQYGGFLRRQFKVNA

SEQ ID NO: 72 - protein, 19 aa, Artificial Sequence
  Integrated protease cleavage site-binding domain (Rimorphin)
EXXYXQYGGFLRRHFKISV

SEQ ID NO: 73 - protein, 19 aa, Artificial Sequence
  Integrated protease cleavage site-binding domain (Rimorphin)
EXXYXQYGGFLRRHFKITV

SEQ ID NO: 74 - protein, 19 aa, Artificial Sequence
  Integrated protease cleavage site-binding domain (Rimorphin)
EXXYXQYGGFLRRHYKLSV

SEQ ID NO: 75 - protein, 23 aa, Artificial Sequence
  Integrated protease cleavage site-binding domain (Nociceptin)
EXXYXQFGGFTGARKSARKRKNQ

SEQ ID NO: 76 - protein, 23 aa, Artificial Sequence
  Integrated protease cleavage site-binding domain (Nociceptin)
EXXYXQFGGFYGARKSARKLANQ

SEQ ID NO: 77 - protein, 23 aa, Artificial Sequence
  Integrated protease cleavage site-binding domain (Nociceptin)
EXXYXQFGGFTGARKSARKYANQ

SEQ ID NO: 78 - protein, 19 aa, Artificial Sequence
  Integrated protease cleavage site-binding domain (Nociceptin)
EXXYXQFGGFTGARKSARK

SEQ ID NO: 79 - protein, 19 aa, Artificial Sequence
  Integrated protease cleavage site-binding domain (Nociceptin)
EXXYXQFGGFTGARKYARK

SEQ ID NO: 80 - protein, 19 aa, Artificial Sequence
  Integrated protease cleavage site-binding domain (Nociceptin)
EXXYXQFGGFTGARKSYRK

SEQ ID NO: 81 - protein, 17 aa, Artificial Sequence
  Integrated protease cleavage site-binding domain (Nociceptin)
EXXYXQFGGFTGARKSA

SEQ ID NO: 82 - protein, 17 aa, Artificial Sequence
  Integrated protease cleavage site-binding domain (Nociceptin)
EXXYXQFGGFTGARKYA

SEQ ID NO: 83 - protein, 17 aa, Artificial Sequence
  Integrated protease cleavage site-binding domain (Nociceptin)
EXXYXQFGGFTGARKSY

SEQ ID NO: 84 - protein, 15 aa, Artificial Sequence
  Integrated protease cleavage site-binding domain (Nociceptin)
EXXYXQFGGFTGARK

SEQ ID NO: 85 - protein, 36 aa, Artificial Sequence
  Integrated protease cleavage site-binding domain (Neuropeptide)
EXXYXQMPRVRSLFQEQEEPEPGMEEAGEMEQKQLQ

SEQ ID NO: 86 - protein, 23 aa, Artificial Sequence
  Integrated protease cleavage site-binding domain (Neuropeptide)
EXXYXQFSEFMRQYLVLSMQSSQ

SEQ ID NO: 87 - protein, 14 aa, Artificial Sequence
  Integrated protease cleavage site-binding domain (Neuropeptide)
EXXYXQTLHQNGNV

SEQ ID NO: 88 - protein, 12 aa, Artificial Sequence
  Integrated protease cleavage site-binding domain (PAR1)
EXXYXQSFLLRN

SEQ ID NO: 89 - protein, 12 aa, Artificial Sequence
  Integrated protease cleavage site-binding domain (PAR1)
EXXYXQSFFLRN

SEQ ID NO: 90 - protein, 12 aa, Artificial Sequence
  Integrated protease cleavage site-binding domain (PAR1)
EXXYXQSFFLKN

SEQ ID NO: 91 - protein, 12 aa, Artificial Sequence
  Integrated protease cleavage site-binding domain (PAR1)
EXXYXQTFLLRN

SEQ ID NO: 92 - protein, 12 aa, Artificial Sequence
  Integrated protease cleavage site-binding domain (PAR1)
EXXYXQGFPGKF

SEQ ID NO: 93 - protein, 12 aa, Artificial Sequence
  Integrated protease cleavage site-binding domain (PAR1)
EXXYXQGYPAKF

SEQ ID NO: 94 - protein, 12 aa, Artificial Sequence
  Integrated protease cleavage site-binding domain (PAR1)
EXXYXQGYPLKF

SEQ ID NO: 95 - protein, 12 aa, Artificial Sequence
  Integrated protease cleavage site-binding domain (PAR1)
EXXYXQGYPIKF

SEQ ID NO: 96 - protein, 12 aa, Artificial Sequence
  Integrated protease cleavage site-binding domain (PAR2)
EXXYXQSLIGKV

SEQ ID NO: 97 - protein, 12 aa, Artificial Sequence
  Integrated protease cleavage site-binding domain (PAR2)
EXXYXQSLIGRL

SEQ ID NO: 98 - protein, 12 aa, Artificial Sequence
  Integrated protease cleavage site-binding domain (PAR3)
EXXYXQTFRGAP

SEQ ID NO: 99 - protein, 12 aa, Artificial Sequence
  Integrated protease cleavage site-binding domain (PAR3)
EXXYXQSFNGGP

SEQ ID NO: 100 - protein, 12 aa, Artificial Sequence
  Integrated protease cleavage site-binding domain (PAR3)
EXXYXQSFNGNE

SEQ ID NO: 101 - protein, 12 aa, Artificial Sequence
  Integrated protease cleavage site-binding domain (PAR4)
EXXYXQGYPGQV

SEQ ID NO: 102 - protein, 12 aa, Artificial Sequence
  Integrated protease cleavage site-binding domain (PAR4)
EXXYXQAYPGKF

SEQ ID NO: 103 - protein, 12 aa, Artificial Sequence
  Integrated protease cleavage site-binding domain (PAR4)
EXXYXQTYPGKF

SEQ ID NO: 104 - protein, 12 aa, Artificial Sequence
  Integrated protease cleavage site-binding domain (PAR4)
EXXYXQGYPGKY

SEQ ID NO: 105 - protein, 12 aa, Artificial Sequence
  Integrated protease cleavage site-binding domain (PAR4)
EXXYXQGYPGKW

SEQ ID NO: 106 - protein, 12 aa, Artificial Sequence
  Integrated protease cleavage site-binding domain (PAR4)
EXXYXQGYPGKK

SEQ ID NO: 107 - protein, 12 aa, Artificial Sequence
  Integrated protease cleavage site-binding domain (PAR4)
EXXYXQGYPGKF

SEQ ID NO: 108 - protein, 12 aa, Artificial Sequence
  Integrated protease cleavage site-binding domain (PAR4)
EXXYXQGYPGRF

SEQ ID NO: 109 - protein, 12 aa, Artificial Sequence
  Integrated protease cleavage site-binding domain (PAR4)
EXXYXQGYPGFK

SEQ ID NO: 110 - protein, 12 aa, Artificial Sequence
  Integrated protease cleavage site-binding domain (PAR4)
EXXYXQGYPAKF

SEQ ID NO: 111 - protein, 12 aa, Artificial Sequence
  Integrated protease cleavage site-binding domain (PAR4)
EXXYXQGFPGKF

SEQ ID NO: 112 - protein, 12 aa, Artificial Sequence
  Integrated protease cleavage site-binding domain (PAR4)
EXXYXQGFPGKP

SEQ ID NO: 113 - protein, 12 aa, Artificial Sequence
  Integrated protease cleavage site-binding domain (PAR4)
EXXYXQSYPGKF

SEQ ID NO: 114 - protein, 12 aa, Artificial Sequence
  Integrated protease cleavage site-binding domain (PAR4)
EXXYXQSYPAKF

SEQ ID NO: 115 - protein, 12 aa, Artificial Sequence
  Integrated protease cleavage site-binding domain (PAR4)
EXXYXQSYPGRF

SEQ ID NO: 116 - protein, 12 aa, Artificial Sequence
  Integrated protease cleavage site-binding domain (PAR4)
EXXYXQSYAGKF

SEQ ID NO: 117 - protein, 12 aa, Artificial Sequence
  Integrated protease cleavage site-binding domain (PAR4)
EXXYXQSFPGQP

SEQ ID NO: 118 - protein, 12 aa, Artificial Sequence
  Integrated protease cleavage site-binding domain (PAR4)
EXXYXQSFPGQA

SEQ ID NO: 119 - protein, 5 aa, Artificial Sequence
  P1′-P2′-P3′-P4′-P5′ portion of Human Rhinovirus 3C protease cleavage site
GPXXX

SEQ ID NO: 120 - protein, 5 aa, Artificial Sequence
  P5-P4-P3-P2-P1 portion of Human Rhinovirus 3C protease cleavage site
XXLFQ

SEQ ID NO: 121 - protein, 6 aa, Artificial Sequence
  Integrated protease cleavage site consensus sequence
EXXYXQ

SEQ ID NO: 122 - protein, 6 aa, Artificial Sequence
  Integrated protease pleavage site
ENLYFQ

SEQ ID NO: 123 - protein, 6 aa, Artificial Sequence
  Integrated protease cleavage site
ENIYTQ

SEQ ID NO: 124 - protein, 6 aa, Artificial Sequence
  Integrated protease cleavage site
ENIYLQ

SEQ ID NO: 125 - protein, 6 aa, Artificial Sequence
  Integrated protease cleavage site
ENVYFQ

SEQ ID NO: 126 - protein, 6 aa, Artificial Sequence
  Integrated protease cleavage site
ENVYSQ

SEQ ID NO: 127 - protein, 5 aa, Artificial Sequence
  Integrated protease cleavage site consensus sequence
XVRFQ

SEQ ID NO: 128 - protein, 5 aa, Artificial Sequence
  Integrated protease cleavage site
TVRFQ

SEQ ID NO: 129 - protein, 5 aa, Artificial Sequence
  Integrated protease cleavage site
NVRFQ

SEQ ID NO: 130 - protein, 5 aa, Artificial Sequence
  Integrated protease cleavage site consensus sequence
XDXXD

SEQ ID NO: 131 - protein, 5 aa, Artificial Sequence
  Integrated protease cleavage site
LDEVD

SEQ ID NO: 132 - protein, 5 aa, Artificial Sequence
  Integrated protease cleavage site
VDEPD

SEQ ID NO: 133 - protein, 5 aa, Artificial Sequence
  Integrated protease cleavage site
VDELD

SEQ ID NO: 134 - protein, 1296 aa, Clostridia botulinum serotype A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 135 - protein, 1291 aa, Clostridia botulinum serotype B
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 136 - protein, 1291 aa, Clostridia botulinum serotype C1
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 137 - protein, 1276 aa, Clostridia botulinum serotype D
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 138 - protein, 1252 aa, Clostridia botulinum serotype E
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 139 - protein, 1274 aa, Clostridia botulinum serotype F
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 140 - protein, 1297 aa, Clostridia botulinum serotype G
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 141 - protein, 1315 aa, Clostridia tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 142 - protein, 1268 aa, Clostridia baratii
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYVILLSNYTAIASRLSQVNRNNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLIVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLRAEYNIYSIKEELNKKVSLAMQNIDRFLTESSISYLMKLINEAKINKLSEYDKRVNQYLLNYILENSSTLGTSSVPELNNLVSNTLNNSIPFELSEYTNDKILIHILIRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 143 - protein, 1251 aa, Clostridia butyricum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNCTMNFKNNNGNNIGLLGFKADTVVASTWYYTHMRDNTNSNGFFWNFISEEHGWQEK

SEQ ID NO: 144 - protein, 4 aa, Artificial Sequence
  Flexible G-spacer
GGGG

SEQ ID NO: 145 - protein, 5 aa, Artificial Sequence
  Flexible G-spacer
GGGGS

SEQ ID NO: 146 - protein, 4 aa, Artificial Sequence
  Flexible A-spacer
AAAA

SEQ ID NO: 147 - protein, 5 aa, Artificial Sequence
  Flexible A-spacer
AAAAV

SEQ ID NO: 148 - DNA, 2649 bp, Artificial Sequence
  Modified BoNT/A comprising an enterokinase cleavage site and a nociceptin binding domain
atgccgttcgtaaacaaacagttcaactataaagacccagtcaacggcgtggacattgcctatatcaaaatcccgaatgcgggtcaaatgcagcccgtgaaagcatttaaaatccataacaaaatttgggtgatcccggagcgcgatacgttcacgaacccggaagaaggagatttaaacccaccgcctgaggctaaacaggtcccggtgtcttactatgatagcacatacctgagtaccgacaatgaaaaggacaactacctgaaaggtgttaccaaactgttcgagcgcatttattcgacagatctcggtcgcatgttgctgacttctattgtgcgcggcattccgttttggggtggtagcaccatcgatacagaactcaaagtgattgacaccaactgcatcaatgtgattcagcctgatgggagctaccggtccgaagagcttaacctcgtaatcattggcccgagcgcggatattatccaattcgaatgtaaatcttttgggcatgaagtcctgaatctgacgcggaatggctatggatcgacgcagtatattcgtttttctccagatttcacatttggatttgaagaaagcctcgaagttgatacgaaccctcttttaggcgcgggaaaattcgcgacggacccagcggtgaccttggcacatgaacttattcatgccgggcatcgcttgtatggaatcgccattaacccgaaccgtgttttcaaggtgaatacgaacgcgtattacgagatgtcgggcttagaagtgtcctttgaagaactgcgcacgtttggcggtcatgatgcaaaatttattgatagtctgcaagaaaacgaatttcggctgtactattacaataaattcaaagacattgcatcaaccttaaacaaggcgaaaagcattgtgggtaccacggctagcttacaatatatgaaaaacgttttcaaagaaaaatacctccttagcgaagacacttccggcaaattctctgtcgataaactgaaatttgataaactgtataaaatgctcaccgagatctacacagaggataactttgtcaaattcttcaaggtcttgaatcggaaaacctatctgaacttcgataaagccgtctttaagatcaacatcgtaccgaaagttaactacaccatctatgatggctttaatctgcgcaatacgaatctggcggcgaactttaacggccagaacaccgaaatcaacaacatgaactttactaaactgaaaaattttaccggcttgtttgaattctataagctcctgtgtgtccgcggtattatcaccagcaaaaccaaatccttgggcggtggtggcgaaaacctgtacttccagggcggtggcggtggtgataagggctataacaaggccttcaatgatttatgcatcaaggtgaacaactgggacttgtttttctctccatctgaagataattttactaacgacttgaacaaaggagaggaaattacttccgataccaacatcgaagcagcggaagagaatattagtctagatcttattcaacaatattacctgacctttaattttgataacgagcctgagaacatttccattgagaatctcagctctgacatcatcggccagctggaactgatgccgaatatcgaacgctttcctaatggaaagaaatatgaattggacaaatacaccatgttccactatctccgcgcgcaggagtttgagcacggcaagtctcgtattgctctgaccaattcggtaaacgaagcccttttaaatccttcgcgtgtgtacacctttttctcaagcgattatgttaaaaaagtgaacaaggcgaccgaagcggcgatgtttttgggatgggtggaacaactggtatatgactttacggatgaaacttctgaagtctcgaccaccgacaaaattgccgatattaccattatcattccctatattggccctgcactgaacattggtaacatgctgtataaagatgattttgtgggcgccctgatcttttcaggcgctgttatcctgctggaatttatcccggaaatcgccattccagtactcggtacctttgcgctggtgtcctatatcgcaaacaaagttttgactgtccagacgatcgacaacgcgctcagtaaacgtaacgaaaaatgggatgaggtgtataagtatattgttaccaactggctcgctaaagtaaacacccagattgacctgattcgcaagaagatgaaagaagcgctggaaaaccaagcagaagcgaccaaagctattatcaactatcaatataaccagtacacagaggaagaaaagaataacatcaacttcaacatcgacgacttatcttcaaagctgaatgaatctattaacaaagcgatgattaatattaacaagttcttgaaccaatgtagtgtcagctatctgatgaactcgatgatcccttacggtgtgaaacgtctggaagacttcgatgcaagccttaaagatgcccttctgaagtatatttacgataatcgcggaactcttattggccaagtggatcgcttaaaagataaagtcaacaacacgctgagtacagacatcccttttcagctgtctaaatatgtggacaatcagcgccaccatcaccatcaccactaa

SEQ ID NO: 149 - DNA, 323 bp, Artificial Sequence
  Fragment encoding integrated protease cleavage site-nociceptin binding domain
gaattctacaagctgctgtgcgtcgacggcatcattacctccaaaactaaatctgaaaacctgtacttccagtttggcggtttcacgggcgcacgcaaatcagcgcgtaaacgtaagaaccaggcgctagcgggcggtggcggtagcggcggtggcggtagcggcggtggcggtagcgcactagtgctgcagtgtatcaaggttaacaactgggatttattcttcagcccgagtgaagacaacttcaccaacgacctgaacaaaggtgaagaaatcacctcagatactaacatcgaagcagccgaagaaaacatcagtctaga

SEQ ID NO: 150 - DNA, 2706 bp, Artificial Sequence
  Modified BoNT/A comprising an integrated protease cleavage site-nociceptin binding domain
atgccgttcgtaaacaaacagttcaactataaagacccagtcaacggcgtggacattgcctatatcaaaatcccgaatgcgggtcaaatgcagcccgtgaaagcatttaaaatccataacaaaatttgggtgatcccggagcgcgatacgttcacgaacccggaagaaggagatttaaacccaccgcctgaggctaaacaggtcccggtgtcttactatgatagcacatacctgagtaccgacaatgaaaaggacaactacctgaaaggtgttaccaaactgttcgagcgcatttattcgacagatctcggtcgcatgttgctgacttctattgtgcgcggcattccgttttggggtggtagcaccatcgatacagaactcaaagtgattgacaccaactgcatcaatgtgattcagcctgatgggagctaccggtccgaagagcttaacctcgtaatcattggcccgagcgcggatattatccaattcgaatgtaaatcttttgggcatgaagtcctgaatctgacgcggaatggctatggatcgacgcagtatattcgtttttctccagatttcacatttggatttgaagaaagcctcgaagttgatacgaaccctcttttaggcgcgggaaaattcgcgacggacccagcggtgaccttggcacatgaacttattcatgccgggcatcgcttgtatggaatcgccattaacccgaaccgtgttttcaaggtgaatacgaacgcgtattacgagatgtcgggcttagaagtgtcctttgaagaactgcgcacgtttggcggtcatgatgcaaaatttattgatagtctgcaagaaaacgaatttcggctgtactattacaataaattcaaagacattgcatcaaccttaaacaaggcgaaaagcattgtgggtaccacggctagcttacaatatatgaaaaacgttttcaaagaaaaatacctccttagcgaagacacttccggcaaattctctgtcgataaactgaaatttgataaactgtataaaatgctcaccgagatctacacagaggataactttgtcaaattcttcaaggtcttgaatcggaaaacctatctgaacttcgataaagccgtctttaagatcaacatcgtaccgaaagttaactacaccatctatgatggctttaatctgcgcaatacgaatctggcggcgaactttaacggccagaacaccgaaatcaacaacatgaactttactaaactgaaaaattttaccggcttgtttgaattctacaagctgctgtgcgtcgacggcatcattacctccaaaactaaatctgaaaacctgtacttccagtttggcggtttcacgggcgcacgcaaatcagcgcgtaaacgtaagaaccaggcgctagcgggcggtggcggtagcggcggtggcggtagcggcggtggcggtagcgcactagtgctgcagtgtatcaaggttaacaactgggatttattcttcagcccgagtgaagacaacttcaccaacgacctgaacaaaggtgaagaaatcacctcagatactaacatcgaagcagccgaagaaaacatcagtctagatcttattcaacaatattacctgacctttaattttgataacgagcctgagaacatttccattgagaatctcagctctgacatcatcggccagctggaactgatgccgaatatcgaacgctttcctaatggaaagaaatatgaattggacaaatacaccatgttccactatctccgcgcgcaggagtttgagcacggcaagtctcgtattgctctgaccaattcggtaaacgaagcccttttaaatccttcgcgtgtgtacacctttttctcaagcgattatgttaaaaaagtgaacaaggcgaccgaagcggcgatgtttttgggatgggtggaacaactggtatatgactttacggatgaaacttctgaagtctcgaccaccgacaaaattgccgatattaccattatcattccctatattggccctgcactgaacattggtaacatgctgtataaagatgattttgtgggcgccctgatcttttcaggcgctgttatcctgctggaatttatcccggaaatcgccattccagtactcggtacctttgcgctggtgtcctatatcgcaaacaaagttttgactgtccagacgatcgacaacgcgctcagtaaacgtaacgaaaaatgggatgaggtgtataagtatattgttaccaactggctcgctaaagtaaacacccagattgacctgattcgcaagaagatgaaagaagcgctggaaaaccaagcagaagcgaccaaagctattatcaactatcaatataaccagtacacagaggaagaaaagaataacatcaacttcaacatcgacgacttatcttcaaagctgaatgaatctattaacaaagcgatgattaatattaacaagttcttgaaccaatgtagtgtcagctatctgatgaactcgatgatcccttacggtgtgaaacgtctggaagacttcgatgcaagccttaaagatgcccttctgaagtatatttacgataatcgcggaactcttattggccaagtggatcgcttaaaagataaagtcaacaacacgctgagtacagacatcccttttcagctgtctaaatatgtggacaatcagcgccaccatcaccatcaccactaa

SEQ ID NO: 151 - protein, 901 aa, Artificial Sequence
  Modified BoNT/A comprising an integrated protease cleavage site-nociceptin binding domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSENLYFQFGGFTGARKSARKRKNQALAGGGGSGGGGSGGGGSALVLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRHHHHHH

SEQ ID NO: 152 - protein, 23 aa, Artificial Sequence
  Integrated protease cleavage site-nociceptin binding domain
ENLYFQFGGFTGARKSARKRKNQ

SEQ ID NO: 153 - protein, 895 aa, Artificial Sequence
  Modified BoNT/A comprising an integrated protease cleavage site-nociceptin binding domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSENLYFQFGGFTGARKSARKRKNQALAGGGGSGGGGSGGGGSALVLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQR

SEQ ID NO: 154 - protein, 5 aa, Homo sapiens
YGGFL

SEQ ID NO: 155 - protein, 5 aa, Homo sapiens
YGGFM

SEQ ID NO: 156 - protein, 8 aa, Homo sapiens
YGGFMRGL

SEQ ID NO: 157 - protein, 7 aa, Homo sapiens
YGGFMRF

SEQ ID NO: 158 - protein, 22 aa, Homo sapiens
YGGFMRRVGRPEWWMDYQKRYG

SEQ ID NO: 159 - protein, 4 aa, Homo sapiens
YPWF

SEQ ID NO: 160 - protein, 4 aa, Homo sapiens
YPFF

SEQ ID NO: 161 - protein, 16 aa, Homo sapiens
YGGFMTSEKSQTPLVT

SEQ ID NO: 162 - protein, 10 aa, Homo sapiens
YGGFLRKYPK

SEQ ID NO: 163 - protein, 31 aa, Homo sapiens
YGGFMTSEKSQTPLVTLFKNAIIKNAYKKGE

SEQ ID NO: 164 - protein, 31 aa, Homo sapiens
YGGFMSSEKSQTPLVTLFKNAIIKNAHKKGQ

SEQ ID NO: 165 - protein, 9 aa, Homo sapiens
YGGFLRKYP

SEQ ID NO: 166 - protein, 17 aa, Homo sapiens
YGGFMTSEKSQTPLVTL

SEQ ID NO: 167 - protein, 17 aa, Homo sapiens
YGGFLRRIRPKLKWDNQ

SEQ ID NO: 168 - protein, 13 aa, Homo sapiens
YGGFLRRIRPKLK

SEQ ID NO: 169 - protein, 16 aa, Homo sapiens
GGFLRRIRPKLKWDNQ

SEQ ID NO: 170 - protein, 12 aa, Homo sapiens
GGFLRRIRPKLK

SEQ ID NO: 171 - protein, 29 aa, Homo sapiens
YGGFLRRQFKVVTRSQEDPNAYSGELFDA

SEQ ID NO: 172 - protein, 13 aa, Homo sapiens
YGGFLRRQFKVVT

SEQ ID NO: 173 - protein, 17 aa, Homo sapiens
FGGFTGARKSARKRKNQ

SEQ ID NO: 174 - protein, 17 aa, Homo sapiens
FGGFTGARKSARKLANQ

SEQ ID NO: 175 - protein, 17 aa, Homo sapiens
FGGFTGARKSARKYANQ

SEQ ID NO: 176 - protein, 11 aa, Homo sapiens
FGGFTGARKSA

SEQ ID NO: 177 - protein, 11 aa, Homo sapiens
FGGFTGARKYA

SEQ ID NO: 178 - protein, 11 aa, Homo sapiens
FGGFTGARKSY

SEQ ID NO: 179 - protein, 13 aa, Homo sapiens
FGGFTGARKSARK

SEQ ID NO: 180 - protein, 30 aa, Homo sapiens
MPRVRSLFQEQEEPEPGMEEAGEMEQKQLQ

SEQ ID NO: 181 - protein, 17 aa, Homo sapiens
FSEFMRQYLVLSMQSSQ

SEQ ID NO: 182 - protein, 8 aa, Homo sapiens
TLHQNGNV

SEQ ID NO: 183 - protein, 6 aa, Artificial Sequence
  Hexapeptide comprising the tethered ligand of PAR1
SFFLRN

SEQ ID NO: 184 - protein, 6 aa, Artificial Sequence
  Hexapeptide comprising the tethered ligand of PAR2
SLIGKV

SEQ ID NO: 185 - protein, 6 aa, Artificial Sequence
  Hexapeptide comprising the tethered ligand of PAR3
TFRGAP

SEQ ID NO: 186 - protein, 6 aa, Artificial Sequence
  Hexapeptide comprising the tethered ligand of PAR4
GYPGQV

SEQ ID NO: 187 - DNA, 311 bp, Artificial Sequence
  Fragment encoding integrated protease cleavage site-nociceptin binding domain
gaattctacaagctgctgtgcgtcgacggcggtggcggtagcgcagaaaacctgtacttccagggctggactttgaactctgctggttatctcctgggcccacatgcggttgctcttgctggtggcggtggctctggcggtggcggtagcggcggtggcggttctgcactagtgcttcagtgtatcaaggttaacaactgggatttattcttcagcccgagtgaagacaacttcaccaacgacctgaacaaaggtgaagaaatcacctcagatactaacatcgaagcagccgaagaaaacatcagtctaga

SEQ ID NO: 188 - protein, 22 aa, Artificial Sequence
  Integrated protease cleavage site-galanin binding domain
ENLYFQGWTLNSAGYLLGPHAV

SEQ ID NO: 189 - DNA, 2727 bp, Artificial Sequence
  Open reading frame for modified BoNT/A comprising an integrated protease cleavage site-galanin binding domain
atgccgttcgtaaacaaacagttcaactataaagacccagtcaacggcgtggacattgcctatatcaaaatcccgaatgcgggtcaaatgcagcccgtgaaagcatttaaaatccataacaaaatttgggtgatcccggagcgcgatacgttcacgaacccggaagaaggagatttaaacccaccgcctgaggctaaacaggtcccggtgtcttactatgatagcacatacctgagtaccgacaatgaaaaggacaactacctgaaaggtgttaccaaactgttcgagcgcatttattcgacagatctcggtcgcatgttgctgacttctattgtgcgcggcattccgttttggggtggtagcaccatcgatacagaactcaaagtgattgacaccaactgcatcaatgtgattcagcctgatgggagctaccggtccgaagagcttaacctcgtaatcattggcccgagcgcggatattatccaattcgaatgtaaatcttttgggcatgaagtcctgaatctgacgcggaatggctatggatcgacgcagtatattcgtttttctccagatttcacatttggatttgaagaaagcctcgaagttgatacgaaccctcttttaggcgcgggaaaattcgcgacggacccagcggtgaccttggcacatgaacttattcatgccgggcatcgcttgtatggaatcgccattaacccgaaccgtgttttcaaggtgaatacgaacgcgtattacgagatgtcgggcttagaagtgtcctttgaagaactgcgcacgtttggcggtcatgatgcaaaatttattgatagtctgcaagaaaacgaatttcggctgtactattacaataaattcaaagacattgcatcaaccttaaacaaggcgaaaagcattgtgggtaccacggctagcttacaatatatgaaaaacgttttcaaagaaaaatacctccttagcgaagacacttccggcaaattctctgtcgataaactgaaatttgataaactgtataaaatgctcaccgagatctacacagaggataactttgtcaaattcttcaaggtcttgaatcggaaaacctatctgaacttcgataaagccgtctttaagatcaacatcgtaccgaaagttaactacaccatctatgatggctttaatctgcgcaatacgaatctggcggcgaactttaacggccagaacaccgaaatcaacaacatgaactttactaaactgaaaaattttaccggcttgtttgaattctacaagctgctgtgcgtcgacggcggtggcggtagcgcagaaaacctgtacttccagggctggactttgaactctgctggttatctcctgggcccacatgcggttgctcttgctggtggcggtggctctggcggtggcggtagcggcggtggcggttctgcactagtgcttcagtgtatcaaggttaacaactgggatttattcttcagcccgagtgaagacaacttcaccaacgacctgaacaaaggtgaagaaatcacctcagatactaacatcgaagcagccgaagaaaacatcagtctagatcttattcaacaatattacctgacctttaattttgataacgagcctgagaacatttccattgagaatctcagctctgacatcatcggccagctggaactgatgccgaatatcgaacgctttcctaatggaaagaaatatgaattggacaaatacaccatgttccactatctccgcgcgcaggagtttgagcacggcaagtctcgtattgctctgaccaattcggtaaacgaagcccttttaaatccttcgcgtgtgtacacctttttctcaagcgattatgttaaaaaagtgaacaaggcgaccgaagcggcgatgtttttgggatgggtggaacaactggtatatgactttacggatgaaacttctgaagtctcgaccaccgacaaaattgccgatattaccattatcattccctatattggccctgcactgaacattggtaacatgctgtataaagatgattttgtgggcgccctgatcttttcaggcgctgttatcctgctggaatttatcccggaaatcgccattccagtactcggtacctttgcgctggtgtcctatatcgcaaacaaagttttgactgtccagacgatcgacaacgcgctcagtaaacgtaacgaaaaatgggatgaggtgtataagtatattgttaccaactggctcgctaaagtaaacacccagattgacctgattcgcaagaagatgaaagaagcgctggaaaaccaagcagaagcgaccaaagctattatcaactatcaatataaccagtacacagaggaagaaaagaataacatcaacttcaacatcgacgacttatcttcaaagctgaatgaatctattaacaaagcgatgattaatattaacaagttcttgaaccaatgtagtgtcagctatctgatgaactcgatgatcccttacggtgtgaaacgtctggaagacttcgatgcaagccttaaagatgcccttctgaagtatatttacgataatcgcggaactcttattggccaagtggatcgcttaaaagataaagtcaacaacacgctgagtacagacatcccttttcagctgtctaaatatgtggacaatcagcgcctgctgtccacgcttgaagcactggcttctggtcaccatcaccatcaccactaa

SEQ ID NO: 190 - protein, 908 aa, Artificial Sequence
  Modified BoNT/A comprising an integrated protease cleavage site-galanin binding domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGGGGSAENLYFQGWTLNSAGYLLGPHAVALAGGGGSGGGGSGGGGSALVLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTLEALASGHHHHHH

SEQ ID NO: 191 - protein, 36 aa, Artificial Sequence
  Integrated protease cleavage site-galanin binding domain consenus sequence
EXXYXQGWTLNSAGYLLGPHAVGNHRSFSDKNGLTS

SEQ ID NO: 192 - protein, 26 aa, Artificial Sequence
  Integrated protease cleavage site-galanin binding domain consenus sequence
EXXYXQGWTLNSAGYLLGPHAVGNHR

SEQ ID NO: 193 - protein, 22 aa, Artificial Sequence
  Integrated protease cleavage site-galanin binding domain consenus sequence
EXXYXQGWTLNSAGYLLGPHAV

SEQ ID NO: 194 - protein, 21 aa, Artificial Sequence
  Integrated protease cleavage site-galanin binding domain consenus sequence
EXXYXQGWTLNSAGYLLGPHA

SEQ ID NO: 195 - protein, 20 aa, Artificial Sequence
  Integrated protease cleavage site-galanin binding domain consenus sequence
EXXYXQGWTLNSAGYLLGPH

SEQ ID NO: 196 - protein, 18 aa, Artificial Sequence
  Integrated protease cleavage site-galanin binding domain consenus sequence
EXXYXQGWTLNSAGYLLG

SEQ ID NO: 197 - protein, 35 aa, Artificial Sequence
  Integrated protease cleavage site-galanin binding domain consenus sequence
EXXYXQWTLNSAGYLLGPHAVGNHRSFSDKNGLTS

SEQ ID NO: 198 - protein, 33 aa, Artificial Sequence
  Integrated protease cleavage site-galanin binding domain consenus sequence
EXXYXQLNSAGYLLGPHAVGNHRSFSDKNGLTS